SEQUENCE LISTING (6 sequences)

SEQ ID NO: 1 - DNA, 51 bp, synthetic construct
  source 1..51: mol_type = other DNA
atgtggctgcagtctctgctgctgctgggaacagtggcctgtagcatctct

SEQ ID NO: 2 - DNA, 108 bp, synthetic construct
  source 1..108: mol_type = other DNA
ctgatccccatcgctgtgggtggtgccctggcggggctggtcctcatcgtcctcatcgcctacctcgtcggcaggaagaggagtcacgcaggctaccagactatctag

SEQ ID NO: 3 - DNA, 19 bp, synthetic construct
  source 1..19: mol_type = other DNA
taatacgactcactatagg

SEQ ID NO: 4 - DNA, 43 bp, synthetic construct
  source 1..43: mol_type = other DNA
gggagacccaagctggctagcgtttaaacttaagcttgccacc

SEQ ID NO: 5 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 6 - DNA, 10 bp, synthetic construct
  source 1..10: mol_type = other DNA
gccrccatgg

(19) United States
(12) Patent Application Publication
(10) Pub. No.: US 2023/0263888 A1
(43) Pub. Date: Aug. 24, 2023

(54) MONOCYTES INDUCING ANTIGEN-SPECIFIC TOLERANCE, ENGINEERED MONOCYTES, AND METHOD OF USE THEREOF

(71) Applicant: Myeloid Therapeutics, Inc., Cambridge, MA (US)

(72) Inventor: Daniel GETTS, Stow, MA (US)

(21) Appl. No.: 17/958,041

(22) Filed: Sep. 30, 2022

Related U.S. Application Data
(60) Provisional application No. 63/250,415, filed on Sep. 30, 2021.

(51) Int. Cl. A61K 39/00; A61K 38/18; A61K 38/20; A61K 38/17; A61K 39/39; A61K 31/155; A61P 37/06

(52) U.S. Cl. CPC A61K 39/4614; A61K 39/46433; A61K 39/0008; A61K 38/1841; A61K 38/2066; A61K 38/1761; A61K 39/39; A61K 31/155; A61P 37/06; A61K 2039/55516; A61K 2039/5256; A61K 2039/523; A61K 2239/39

(57) ABSTRACT

Methods and compositions for treating autoimmune diseases and conditions using engineered myeloid cells.

### CROSS REFERENCE

The instant application claims priority to U.S. Provisional Application 63/250,415, filed on Sep. 30, 2021, which is fully incorporated herein by reference.

### SEQUENCE LISTING

The instant application contains a Sequence Listing which has been submitted electronically in XML format and is hereby incorporated by reference in its entirety. Said XML copy, created on May 9, 2023, is named 56371-722_201_SL.xml and is 6,698 bytes in size.

### BACKGROUND

Cellular immunotherapy is a promising new technology for fighting difficult to treat diseases, such as cancer, and persistent infections and also certain diseases that are refractory to other forms of treatment. To date, the majority of therapies approved by the US Food and Drug Administration (FDA) for auto-immune diseases have focused on the global inhibition of immune inflammatory activity. The treatment of autoimmune diseases requires life-long use of general immunosuppressants or immunomodulators that may target-specific pathways (e.g., TNF-α) but are not antigen-specific. A long-standing goal for immunotherapy is the development of antigen-specific therapies that leave the rest of the immune system intact and that can arrest or even reverse disease pathology (Front Immunol. 2018; 9: 230).

The goal of ongoing research for treatment of autoimmune diseases is the development of autoimmune antigen specific treatments that allow for the specific blockade of the deleterious effects of self-reactive immune cell function, while maintain-ing the ability of the immune system to clear non-self-antigens. Molecular mechanisms described for antigen-specific therapies are complex and not completely understood (Adv Drug Deliv Rev. 2017 May 15; 114: 240-255). The majority of efforts for the development of therapeutics have focused on inducing tolerogenic responses through antigen presenting cells (APCs) or lymphocyte reprogramming. Modulation of signal 1 and signal 2 of T cell activation through APCs represents an important pathway to alter T cell activation and induce tolerance. Other mechanisms such as direct interactions of therapeutics and CAR T cells with lymphocytes to induce tolerogenic phenotypes such as Tregs and deletion represent additional methods to curb aberrant immune activation. However, as each technology is different, individual mechanisms of action need to be identified and should not be extrapolated from one to another (Adv Drug Deliv Rev. 2017 May 15; 114: 240-255).

Developing improved strategies to produce therapeutics that deliver peptide and protein antigens will be required to enable the large-scale progression of these technologies to the clinic. Particle-based therapeutics have shown some promise in the field, however, difficulties regarding scale up and controlling physicochemical properties such as size, charge, and antigen release as well as methods to characterize the presence of relevant epitopes within the particle will need to be overcome (Adv Drug Deliv Rev. 2017 May 15; 114: 240-255)

Accordingly, there is a need for improved and novel approaches that are capable of efficiently inducing long-term immune tolerance without the need for administration of high initial doses of immunosuppressive drugs, or the use of particle-based therapeutics. The present invention addresses this need and provides related advantages as well.

### SUMMARY

In one aspect, provided herein is a population of human monocytes comprising an effective amount of a recombinant nucleic acid encoding a human autoimmune antigen. In one aspect, provided herein is a population of human monocytes comprising an effective amount a recombinant human autoimmune antigen. In one aspect, provided herein is a population of apoptotic human monocytes comprising an effective amount of a recombinant human autoimmune antigen in one or more vesicles. In one aspect, provided herein is a population of apoptotic human monocytes comprising an effective amount a recombinant human autoimmune antigen in one or more vesicles, wherein the human autoimmune antigen is encoded by a recombinant nucleic acid present in the human monocyte.

In some embodiments, the population comprises CD14⁺CD16⁻ human monocytes. In some embodiments, the population comprises CD14^{dim}CD16⁺ human monocytes. In some embodiments, the population comprises CD14⁺CD16⁺ human monocytes. In some embodiments, the population comprises CD14⁻CD16⁻ human monocytes. In some embodiments, the population is substantially free of monocyte-derived dendritic cells. In some embodiments, the population is substantially free of monocyte-derived macrophages. In some embodiments, the recombinant nucleic acid comprises a first nucleic acid sequence encoding a signal peptide, a second nucleic acid sequence encoding the human autoimmune antigen, and a third nucleic acid sequence encoding an immunomodulatory polypeptide. In some embodiments, the recombinant nucleic acid comprises a modification to increase nucleic acid stability and/or expression of the human autoimmune antigen. In some embodiments, the recombinant nucleic acid comprises a viral vector, DNA plasmid or an RNA vector.

In some embodiments, the RNA vector comprises a 5′UTR from a highly expressed gene. In some embodiments, the RNA vector comprises a stabilizing 3′UTR. In some embodiments, the RNA vector comprises a stabilizing 3′UTR from B-globin. In some embodiments, the RNA vector comprises a triplex forming sequence. In some embodiments, the RNA vector comprises a MascRNA-tRNA like sequence. In some embodiments, the RNA vector comprises a flavivirus sfRNA. In some embodiments, the recombinant nucleic acid comprises a signal peptide or a variant thereof.

In some embodiments, the recombinant nucleic acid expresses a fusion polypeptide comprising a human autoimmune antigen and an antigen enhancer. In some embodiments, the antigen enhancer is selected from LAMP-1/2, hsp110 and grp170, hsp70, hsp65, rab7 GTPas, PSGL-1/mIgG_{2b}, macrophage mannose receptor (MMR), and dendritic cell-specific intercellular adhesion molecule-3 grabbing non-integrin (DC-SIGN), or a MHC class I trafficking signal. In some embodiments, the human autoimmune antigen is associated with Celiac Disease. In some embodiments, the human autoimmune antigen is gliadin, a barley protein, or hordein polypeptide or fragment thereof. In some embodiments, the human autoimmune antigen is associated with Multiple Sclerosis. In some embodiments, the human autoimmune antigen is selected from the group consisting of MBP13-32, MBP83-99, MBP111-129, MBP146-170, MOG1-20, MOG35-55, and PLP139-15 polypeptide or fragment thereof. In some embodiments, the human autoimmune antigen is associated with Type 1 Diabetes. In some embodiments, the human autoimmune antigen is selected from the group consisting of insulin or an insulin-like polypeptide, or Islet-Specific Glucose-6-Phosphatase Catalytic Subunit-Related Protein (IGRP) polypeptide or fragment thereof. In some embodiments, the human autoimmune antigen is associated with Myasthenia gravis.

In some embodiments, the RNA vector comprises a first nucleic acid sequence encoding a signal peptide, a second nucleic acid sequence encoding at least two human autoimmune antigens, and a third nucleic acid sequence encoding an immunomodulatory polypeptide, wherein the immunomodulatory polypeptide comprises a Lysosome-Associated Membrane Glycoprotein-1 (LAMP-1) polypeptide or fragment thereof.

In some embodiments, the population comprises a plurality of RNA vectors comprising a plurality of human autoimmune antigens, wherein the human monocytes individually comprise one or more RNA vectors.

In some embodiments, the human monocytes are 1-ethyl-3-(3-dimethylaminopropyl)carbodiimide-treated human monocytes.

In one aspect, provided herein is an RNA vector comprising a first nucleic acid sequence encoding a signal peptide, a second nucleic acid sequence encoding at least one human autoimmune antigen, and a third nucleic acid sequence encoding an immunomodulatory polypeptide. In some embodiments, the RNA vector is introduced into the human monocytes by electroporation. In some embodiments, the human monocytes are post-proliferative. In some embodiments, the human monocytes are pre-apoptotic or apoptotic. In some embodiments, the human monocytes are 1-ethyl-3-(3-dimethylaminopropyl)carbodiimide-treated human monocytes. In some embodiments, the RNA vector is introduced into the human monocytes by electroporation.

In one aspect, provided herein is a population of human monocytes comprising a human autoimmune antigen, wherein the human autoimmune antigen is translated from a recombinant polynucleotide, such as a recombinant mRNA or an RNA vector present in the human monocytes at the time of translation, the recombinant RNA or the RNA vector encoding the human autoimmune antigen. In one embodiment, the population of human monocytes comprising a human autoimmune antigen is further treated with an agent ex vivo. In some embodiments, the agent induces apoptosis in the population of human monocytes comprising a human autoimmune antigen. In some embodiments, the apoptosis-inducing agent comprises 1-ethyl-3-(3-dimethylaminopropyl) carbodiimide.

In one aspect, provided herein is a pharmaceutical composition comprising the population of human monocytes comprising a recombinant nucleic acid encoding a human autoimmune antigen of any one of the embodiments described herein.

In one aspect, provided herein is a pharmaceutical composition comprising the population of any preceding embodiment, in an amount effective to prevent an autoimmune disease in a human subject to whom the pharmaceutical composition is administered.

In one aspect, provided herein is a pharmaceutical composition comprising the population of any preceding embodiment, in an amount effective to treat an autoimmune disease or a symptom thereof in a human subject to whom the pharmaceutical composition is administered.

In one aspect, provided herein is a pharmaceutical composition comprising the population of any preceding embodiment, in an amount effective to prevent a recurrence of an autoimmune disease in a human subject to whom the pharmaceutical composition is administered. In some embodiments, the human monocytes are elutriation-purified human monocytes. In some embodiments, the human monocytes are derived from the human subject. In some embodiments, the human monocytes are derived from a human donor other than the human subject. In some embodiments, the human monocytes are 1-ethyl-3-(3-dimethylaminopropyl)carbodiimide-treated human monocytes. In some embodiments, the apoptosis-inducing agent comprises 1-ethyl-3-(3-dimethylaminopropyl) carbodiimide.

In some embodiments, the population of human of human monocytes is isolated from a biological sample of a human subject. In some embodiments, the biological sample is an apheresis sample.

In one aspect, provided herein is a pharmaceutical composition comprising the recombinant nucleic acid of any preceding embodiment, wherein the recombinant nucleic acid is formulated for delivery to human monocytes.

In one aspect, provided herein is a pharmaceutical composition comprising: (I) (a) an exogenously modified population of monocytes; and (b) a pharmaceutically acceptable excipient, diluent or carrier; wherein the exogenously modified population of monocytes comprises a recombinant polynucleotide encoding a polypeptide comprising the autoantigenic peptide; or (II) (a) a recombinant polynucleotide encoding a polypeptide comprising an autoantigenic peptide; (b) an agent that modifies monocytes of a human subject administered the pharmaceutical composition such that the monocytes of the human subject are phagocytosed, engulfed and/or recognized as apoptotic by APCs of the human subject and (c) a pharmaceutically acceptable excipient, diluent or carrier.

In some embodiments, the exogenously modified population of monocytes are phagocytosed, engulfed and/or recognized as apoptotic by antigen presenting cells (APCs) of human subject administered the pharmaceutical composition. In some embodiments, the autoantigenic peptide is an autoantigenic epitope. In some embodiments, the autoantigenic peptide is unknown at the time of administration. In some embodiments, the polypeptide comprising the autoantigenic peptide is longer than the autoantigenic peptide.

In some embodiments, the exogenously modified population of monocytes do not present the autoantigenic peptide. In some embodiments, the polypeptide comprising the autoantigenic peptide is a full-length protein. In some embodiments, the exogenously modified population of monocytes express the polypeptide comprising the autoantigenic peptide. In some embodiments, the exogenously modified population of monocytes comprises exogenously modified monocytes that are apoptotic. In some embodiments, the recombinant polynucleotide encodes two or more autoantigenic peptides from a single protein.

In some embodiments, the polypeptide comprising the autoantigenic peptide comprises a fusion protein.

In some embodiments, the fusion protein comprises an antigen enhancer selected from the group consisting of LAMP-1/2, hsp110 and grp170, hsp70, hsp65, rab7 GTPas, PSGL-1/mIgG_{2b}, macrophage mannose receptor (MMR), and dendritic cell-specific intercellular adhesion molecule-3 grabbing non-integrin (DC-SIGN) and a MHC class I trafficking signal.

In some embodiments, the polypeptide comprising the autoantigenic peptide lacks a secretory sequence. In some embodiments, the polypeptide comprising the autoantigenic peptide comprises a LAMP1 sequence or a fragment thereof. In some embodiments, the autoantigenic peptide and the LAMP1 sequence or a fragment thereof are separated by a cleavable peptide sequence. In some embodiments, the cleavable peptide sequence is a T2A sequence or a P2A sequence. In some embodiments, the polypeptide comprising the autoantigenic peptide comprises an autoantigenic peptide from a protein selected from the group consisting of Gliadin, Barley, Hordein, MBP, MOG, PLP. Insulin, Pro-insulin, IGRP, Casein, ligand for a drug-neutralizing antibody, Factor VIII. In some embodiments, the autoantigenic peptide is selected from the group consisting of MBP13-32, MBP83-99, MBP111-129, MBP146-170, MOG1-20, MOG35-55, PLP139-15.

In some embodiments, the recombinant polynucleotide is RNA. In some embodiments, the recombinant polynucleotide is mRNA. In some embodiments, the recombinant polynucleotide is DNA. In some embodiments, the recombinant polynucleotide is a vector. In some embodiments, the vector is a viral vector. In some embodiments, the viral vector is a lentiviral vector, an adenoviral vector or an adeno-associated viral vector. In one embodiment, the RNA vector comprises mRNA or circular RNA. In some embodiments, the recombinant polynucleotide is an RNA.

In some embodiments, the RNA vector is introduced into the provided population of human monocytes by electroporation.

In some embodiments, the exogenously modified population of monocytes have been modified with an agent such that monocytes of the exogenously modified population of monocytes are phagocytosed, engulfed and/or recognized as apoptotic by APCs of human subject administered the pharmaceutical composition. In some embodiments, the agent comprises an apoptosis-inducing agent. In some embodiments, the agent comprises a cross-linking agent. In some embodiments, the agent comprises an agent that crosslinks lipids. In some embodiments, the agent comprises an agent that crosslinks a cell membrane. In some embodiments, the agent and the recombinant polynucleotide are encapsulated within a particle. In some embodiments, the agent is specific for monocytes. In some embodiments, the agent does not induce apoptosis of the APCs that phagocytose, engulf and/or recognize the monocytes. In some embodiments, the agent is small molecule or gene. In some embodiments, the agent induces apoptosis of the monocytes of the human subject over time. In some embodiments, the agent is destroyed or rendered non-functional or degraded after the APCs phagocytose, engulf and/or recognize the monocytes.

In some embodiments, the pharmaceutically acceptable excipient, diluent or carrier comprises a particle. In some embodiments, the pharmaceutically acceptable excipient, diluent or carrier comprises a lipid nanoparticle.

In some embodiments, the agent comprises an agent that binds with double-stranded DNA of the myeloid cells. In some embodiments, the agent comprises an agent that induces interchain cross-linking within double stranded DNA. In some embodiments, the agent comprises an agent that intercalates within double stranded DNA. In some embodiments, the agent comprises an agent that inhibits RNA synthesis. In some embodiments, the agent comprises 1-ethyl-3-(3-dimethylaminopropyl)-carbodiimide (EDC). In some embodiments, the agent comprises irradiation.

In some embodiments, the agent comprises an acrylamide, a β-carboline alkaloid, anthracycline, carvacrol, p-cymene, doxorubicin, daunorubicin (DNR), idarubicin (IDA), or camptothecin (CAM), blasticidin, or cycloheximide. In some embodiments, the agent is actinomycin D.

In some embodiments, the exogenously modified population of monocytes is an exogenously modified population of CD14+ cells. In some embodiments, the exogenously modified population of monocytes is an exogenously modified population of CD14+CD16+ cells In some embodiments, the exogenously modified population of monocytes is an exogenously modified population of CD14^{dim}CD16+ cells. In some embodiments, the exogenously modified population of monocytes is an exogenously modified population of CD14−CD16+ cells. In some embodiments, the exogenously modified population of monocytes comprises at least about 1×10\hat{ }6 exogenously modified cells. In some embodiments, the exogenously modified population of monocytes comprises at least about 1×10\hat{ }6 monocytes. In some embodiments, the exogenously modified population of monocytes comprises at least about 1×10\hat{ }6 CD14+CD16+ cells, at least about 1×10\hat{ }6 CD14^{dim}CD16+ cells, at least about 1×10\hat{ }6 CD14−CD16+ cells. In some embodiments, the exogenously modified population of monocytes is an exogenously modified population of dendritic cells.

In some embodiments, the cells of the exogenously modified population of monocytes are Annexin V positive. In some embodiments, the cells of the exogenously modified population of monocytes are PI-negative. In some embodiments, the cells of the exogenously modified population of monocytes are live pre-apoptotic cells and/or apoptotic cells.

In one aspect, provided herein is a method of manufacturing a therapeutic composition for prevention or treatment of a human autoimmune disease, the method comprising the steps of: (i) providing a population of human monocytes isolated from a first human subject, wherein the human monocytes comprise CD14⁺CD16⁻ human monocytes, CD14^{dim}CD16⁺ human monocytes, CD14⁻/CD16⁻, CD14⁺CD16⁺ human monocytes or a combination thereof, (ii) contacting the provided population with an RNA vector comprising a first nucleic acid sequence encoding a human autoimmune antigen under conditions such that the RNA vector is introduced into the provided population of human monocytes to generate an engineered population of human monocytes, (iii) incubating the engineered population for a period of time sufficient to translate the encoded human autoimmune antigen in the population of human monocytes comprising a human autoimmune antigen, wherein the human autoimmune antigen is translated from an RNA vector present in the human monocytes at the time of translation, and (iv) contacting the incubated population with an apoptosis-inducing agent for a period time sufficient to induce apoptosis in at least a portion of the incubated population, thereby manufacturing the therapeutic composition.

In some embodiments, the first nucleic acid sequence encodes a plurality of human autoimmune antigens.

In some embodiments, the apoptosis-inducing agent comprises 1-ethyl-3-(3-dimethylaminopropyl)carbodiimide.

In some embodiments, the RNA vector is introduced into the provided population of human monocytes by electroporation.

In some embodiments, the population of human monocytes are isolated from an apheresis product isolated from the first human subject. In some embodiments, the therapeutic composition is suitable for administration to a second human subject, wherein the first and second human subjects are not the same subject.

In some embodiments, the engineered population is incubated for at least four hours. In some embodiments, the engineered population is incubated for up to about twenty-four hours. In some embodiments, the incubated population is contacted with the apoptosis-inducing agent for at least 5, 10, 15, 20, 30, 40, 50 minutes or 1, 2, 3, 4, 5, 6, 7, 8, 9, 10, 11, 12, 13, 14, 15, 16, 17, 18, 19, 20, 21, 22, or 23 hours or 1, 2, 3, 4, 5, 6, 7 days. In some embodiments, the incubated population is contacted with the apoptosis-inducing agent for a period time to induce apoptosis in at least 5, 10, 15, 20, 25, 30, 35, 40, 45, 50, 55, 60, 65, 70, 75, 80, 85, 90, 95, 96, 97, 98, 99, or greater than 99% of the incubated population. In some embodiments, the manufactured therapeutic composition is stable at a temperature below zero degrees Celsius for at least about one month. In some embodiments, the apoptotic population is formulated in a pharmaceutically acceptable buffer suitable for parenteral administration to a human subject in need thereof.

In one aspect, provided herein is a population of human monocytes comprising an effective amount a recombinant nucleic acid encoding a human, humanized or chimeric tolerizing polypeptide. In some embodiments, the tolerizing polypeptide is effective to induce tolerance in a human subject having or at risk of developing graft versus host disease, host versus graft disease, allergy, or anti-drug antibodies, or a human subject treated with a cellular therapeutic or an enzyme replacement therapy. In one aspect, provided herein is a population of human monocytes comprising an effective amount a recombinant tolerizing polypeptide. In one aspect, provided herein is a population of apoptotic human monocytes comprising an effective amount a recombinant tolerizing polypeptide in one or more vesicles. In one aspect, provided herein is a population of apoptotic human monocytes comprising an effective amount a recombinant tolerizing polypeptide in one or more vesicles, wherein the tolerizing polypeptide is encoded by a recombinant nucleic acid present in the human monocyte. In some embodiments, the recombinant nucleic acid comprises a DNA plasmid or an RNA vector.

In some embodiments, the population comprises CD14⁺CD16⁻ human monocytes. In some embodiments, the population comprises CD14^{dim}CD16⁺ human monocytes. In some embodiments, the population comprises CD14⁺CD16⁺ human monocytes. In some embodiments, the population comprises CD14⁻CD16⁻ human monocytes. In some embodiments, the population of any preceding embodiment is substantially free of monocyte-derived dendritic cells. In some embodiments, the population of any preceding embodiment is substantially free of monocyte-derived macrophages.

In some embodiments, the recombinant nucleic acid comprises a first nucleic acid sequence encoding a signal peptide, a second nucleic acid sequence encoding the tolerizing polypeptide, and a third nucleic acid sequence encoding an immunomodulatory polypeptide.

In some embodiments, the recombinant nucleic acid comprises a modification to increase nucleic acid stability and/or expression of the tolerizing polypeptide. In some embodiments, the RNA vector comprises a 5′UTR from a highly expressed gene. In some embodiments, the RNA vector comprises a stabilizing 3′UTR. In some embodiments, the RNA vector comprises a stabilizing 3′UTR from B-globin. In some embodiments, the RNA vector comprises a triplex forming sequence. In some embodiments, the RNA vector comprises a MascRNA-tRNA like sequence. In some embodiments, the RNA vector comprises a flavivirus sfRNA. In some embodiments, the recombinant nucleic acid comprises a signal peptide or a variant thereof. In some embodiments, the recombinant nucleic acid expresses a fusion polypeptide comprising a tolerizing polypeptide and an antigen enhancer. In some embodiments, the antigen enhancer is selected from LAMP-1/2, hsp110 and grp170, hsp70, hsp65, rab7 GTPas, PSGL-1/mIgG_{2b}, macrophage mannose receptor (MMR), and dendritic cell-specific intercellular adhesion molecule-3 grabbing non-integrin (DC-SIGN), or a MHC class I trafficking signal. In some embodiments, the RNA vector comprises a first nucleic acid sequence encoding a signal peptide, a second nucleic acid sequence encoding at least one tolerizing polypeptide, and a third nucleic acid sequence encoding an immunomodulatory polypeptide. In some embodiments, the immunomodulatory polypeptide comprises a Lysosome-Associated Membrane Glycoprotein-1 (LAMP-1) polypeptide or fragment thereof. In some embodiments, the population comprises a plurality of RNA vectors comprising a plurality of tolerizing polypeptides, wherein the human monocytes individually comprise one or more RNA vectors. In some embodiments, the human monocytes are 1-ethyl-3-(3-dimethylaminopropyl) carbodiimide-treated human monocytes.

In one aspect, provided herein is an RNA vector comprising a first nucleic acid sequence encoding a signal peptide, a second nucleic acid sequence encoding at least one tolerizing polypeptide, and a third nucleic acid sequence encoding an immunomodulatory polypeptide. In another aspect, provided herein is a pharmaceutical composition comprising the recombinant nucleic acid of any preceding embodiment, wherein the recombinant nucleic acid is formulated for delivery to human monocytes.

In one aspect, provided herein is a population of human monocytes comprising a tolerizing polypeptide, wherein the tolerizing polypeptide is translated from an RNA vector present in the human monocytes at the time of translation, the RNA vector encoding the tolerizing polypeptide. In some embodiments, the human monocytes are post-proliferative. In some embodiments, the human monocytes are pre-apoptotic or apoptotic. In some embodiments, the human monocytes are 1-ethyl-3-(3-dimethylaminopropyl)carbodiimide-treated human monocytes. In some embodiments, the RNA vector is introduced into the human monocytes by electroporation.

In one aspect, provided herein is a pharmaceutical composition comprising the population of any preceding embodiments, in an amount effective to prevent an immune-associated disease in a human subject to whom the pharmaceutical composition is administered. In some embodiments, the pharmaceutical composition comprising the population of any preceding embodiments, for administering to a human subject in an amount effective to treat an immune-associated disease or a symptom thereof in the human subject to whom the pharmaceutical composition is administered. In some embodiments, the pharmaceutical composition comprising the population of any preceding embodiment is provided in an amount effective to prevent a recurrence of an immune-associated disease in a human subject to whom the pharmaceutical composition is administered. In some embodiments, the human monocytes are elutriation-purified human monocytes. In some embodiments, the human monocytes are derived from the human subject. In some embodiments, the human monocytes are derived from a human donor other than the human subject. In some embodiments, the human monocytes are 1-ethyl-3-(3-dimethylaminopropyl)carbodiimide-treated human monocytes.

In one aspect, provided herein is a method of manufacturing a therapeutic composition for prevention or treatment of a human immune-associated disease, the method comprising the steps of: (i) providing a population of human monocytes isolated from a first human subject, wherein the human monocytes comprise CD14⁺CD16⁻ human monocytes, CD14^{dim}CD16⁺ human monocytes, CD14⁻/CD16⁻, CD14⁺CD16⁺ human monocytes or a combination thereof, (ii) contacting the provided population with an RNA vector comprising a first nucleic acid sequence encoding a tolerizing polypeptide under conditions such that the RNA vector is introduced into the provided population of human monocytes to generate an engineered population of human monocytes, (iii) incubating the engineered population for a period of time sufficient to translate the encoded tolerizing polypeptide in the population of human monocytes comprising a tolerizing polypeptide, wherein the tolerizing polypeptide is translated from an RNA vector present in the human monocytes at the time of translation, (iv) contacting the incubated population with an apoptosis-inducing agent for a period time sufficient to induce apoptosis in at least a portion of the incubated population, thereby manufacturing the therapeutic composition.

Provided herein is a method of treating or inducing immune tolerance to an autoantigenic peptide in a human subject with an immune-mediated disease or condition, the method comprising administering to the human subject the pharmaceutical composition of any one of embodiments described herein, wherein the pharmaceutical composition comprises a therapeutically effective amount of the exogenously modified population of monocytes. In some embodiments, the immune-mediated disease or condition is an autoimmune disease, an autoinflammatory disease or condition, an allergic condition, a host-versus graft rejection disease.

In some embodiments, the immune-mediated disease or condition is selected from the group consisting of eczema (atopic dermatitis), asthma. In some embodiments, the immune-mediated disease or condition is selected from the group consisting of a food allergy. In some embodiments, the immune-mediated disease or condition is transplant rejection. In some embodiments, the immune-mediated disease or condition is a lysosomal storage disease. In some embodiments, the exogenously modified population of monocytes do not present the autoantigenic peptide at the time of administration. In some embodiments, administering comprises intravenous administration. In some embodiments, administration of the pharmaceutical composition elicits an innate immune response in the subject. In some embodiments, monocytes of the exogenously modified population of monocytes migrate to a splenic marginal zone sinus of the subject after administration of the pharmaceutical composition. In some embodiments, antigen presenting cells (APCs) of the subject engulf or phagocytose monocytes of the exogenously modified population of monocytes after administration of the pharmaceutical composition. In some embodiments, the monocytes of the exogenously modified population of monocytes undergo scavenger receptor-mediated uptake by APCs of the subject after administration of the pharmaceutical composition. In some embodiments, the APCs of the subject present the autoantigenic peptide in complex with an MHC protein after administration of the pharmaceutical composition.

In some embodiments, the MHC protein is an MHC class II protein. In some embodiments, the APCs of the subject secrete a cytokine or growth factor after administration of the pharmaceutical composition. In some embodiments, the cytokine or the growth factor regulates co-stimulatory molecules. In some embodiments, the cytokine or the growth factor is IL-10 and/or TGF beta. In some embodiments, the APCs of the subject increase expression a negative costimulatory molecule after administration of the pharmaceutical composition. In some embodiments, the negative costimulatory molecule comprises PD-L1, CTLA-4 or any combination thereof. In some embodiments, the APCs of the subject express IL-10 receptor. In some embodiments, the APCs of the subject are selected from the group consisting of B cells, macrophages, dendritic cells and a combination thereof. In some embodiments, the macrophages are marginal zone macrophages (MZMs).

In some embodiments, the effector T cells comprising a T cell receptor (TCR) specific to a peptide:MHC complex comprising the autoantigenic peptide undergo apoptosis after administration of the pharmaceutical composition. In some embodiments, the effector T cells comprising a TCR specific to a peptide:MHC complex comprising the autoantigenic peptide differentiate into anergic T cells after administration of the pharmaceutical composition. In some embodiments, the regulatory T cells (Tregs) of the subject comprising a TCR specific to a peptide:MHC complex comprising the autoantigenic peptide are upregulated after administration of the pharmaceutical composition.

In some embodiments, naive T cells of the subject differentiate into Tregs after administration of the pharmaceutical composition. In some embodiments, naive T cells of the subject comprising a TCR specific to a peptide:MHC complex comprising the autoantigenic peptide differentiate into anergic T cells after administration of the pharmaceutical composition.

In some embodiments, the T cells are CD4+ T cells. In some embodiments, the exogenously modified population of monocytes comprises exogenously modified monocytes that are phagocytosed, engulfed and/or recognized as apoptotic by APCs of the subject.

In some embodiments, the method further comprises modifying a population of monocytes, thereby generating the exogenously modified population of monocytes. In some embodiments, modifying comprises introducing the recombinant polynucleotide into the population of monocytes. In some embodiments, introducing comprises electroporating, transfecting, nucleofecting or transducing. In some embodiments, modifying comprises culturing the population of monocytes ex vivo. In some embodiments, culturing comprises inducing maturation, differentiation or apoptosis of the population of monocytes. In some embodiments, the population of monocytes is an allogeneic or autologous population of monocytes. In some embodiments, modifying comprises contacting a population of monocytes with an agent that modifies the monocytes such that APCs of the human subject phagocytose or engulf exogenously modified monocytes of the exogenously modified population of monocytes.

In some embodiments, the method comprises obtaining, isolating or enriching a population of monocytes from a biological sample from a human subject. In some embodiments, the biological sample is a peripheral blood mononuclear cell (PBMC) sample or a leukapheresis.

Provided herein is a pharmaceutical composition comprising: (I) (a) an exogenously modified population of monocytes; and (b) a pharmaceutically acceptable excipient, diluent or carrier; wherein the exogenously modified population of monocytes comprises a recombinant polynucleotide comprising a first sequence encoding one or more autoantigenic peptides; and one or more additional sequences encoding one or more immune regulatory agents; or, (II) (a) a recombinant polynucleotide encoding a polypeptide comprising a first sequence encoding one or more autoantigenic peptides, and one or more additional sequences encoding one or more immune regulatory agents; (b) an agent that modifies monocytes of a human subject when the pharmaceutical composition is administered such that the monocytes of the human subject are phagocytosed, engulfed and/or recognized as apoptotic by APCs of the human subject and (c) a pharmaceutically acceptable excipient, diluent or carrier. In some embodiments, the autoantigenic peptide is unknown at the time of administration. In some embodiments, the one or more immune regulatory agents comprises TGF beta. In some embodiments, the one or more immune regulatory agents comprises IL10. In some embodiments, the one or more immune regulatory agents comprises PD1. In some embodiments, the one or more immune regulatory agents comprises PDL1. In some embodiments, the agent that modifies monocytes of a human subject comprises TGF beta. In some embodiments, the agent that modifies monocytes of a human subject comprises IL10. In some embodiments, the agent that modifies monocytes of a human subject comprises PD-1. In some embodiments, the agent that modifies monocytes of a human subject comprises PDL1.

In some embodiments, a method of treating an autoimmune disease or disorder is provided herein, wherein the method comprises administering to the subject in need thereof a composition comprising a recombinant polynucleotide encoding (i) one of more antigenic epitopes, and one or more immune regulatory agents comprising (ii) TGF-beta, and (iii) PD1 or PDL1. In some embodiments, a method of treating an autoimmune disease or disorder is provided herein, wherein the method comprises administering to the subject in need thereof a composition comprising a myeloid cell population, comprising engineered myeloid cells comprising recombinant polynucleotide encoding (i) one of more antigenic epitopes, and encoding one or more immune regulatory agents comprising (ii) TGF-beta, and (iii) PD1 or PDL1. In some embodiments, the myeloid cell population may be further comprise or is treated with an agent that induces apoptosis.

In some embodiments, a method of treating an autoimmune disease or disorder is provided herein, wherein the method comprises administering to the subject in need thereof a composition comprising a recombinant polynucleotide encoding (i) one of more antigenic epitopes, and one or more immune regulatory agents comprising (ii) IL10, and (iii) PD1 or PDL1. In some embodiments, a method of treating an autoimmune disease or disorder is provided herein, wherein the method comprises administering to the subject in need thereof a composition comprising a myeloid cell population, comprising engineered myeloid cells comprising recombinant polynucleotide encoding (i) one of more antigenic epitopes, and encoding one or more immune regulatory agents comprising (ii) IL10, and (iii) PD1 or PDL1. In some embodiments, the myeloid cell population may be further comprise or is treated with an agent that induces apoptosis.

In some embodiments, the human subject has T cells with a TCR that is specific to a peptide:MHC complex comprising the autoantigenic peptide. In some embodiments, the method further comprising administering an additional therapeutic to the subject. In some embodiments, the additional therapeutic comprises an immunosuppressive agent.

In some embodiments, the pharmaceutical composition is administered more than once. In some embodiments, the pharmaceutical composition is administered periodically at an interval of 2 months, 3 months, 4 months, 5 months, 6 months, 8 months, 10 months or 12 months. In some embodiments, the human subject is positive for an HLA allele that specifically binds to the autoantigenic peptide. In some embodiments, the method comprises prior to administering the pharmaceutical composition, selecting a human subject that is positive for an HLA allele that specifically binds to the autoantigenic peptide.

In some embodiments, the immune-mediated disease or condition is selected from the group consisting of multiple sclerosis (MS), rheumatoid arthritis, systemic lupus erythematosus (lupus), inflammatory bowel disease (IBD), ulcerative colitis, Crohn's disease, type 1 diabetes mellitus, Guillain-Barre syndrome, chronic inflammatory demyelinating polyneuropathy, psoriasis, Graves' disease, Hashimoto's thyroiditis, myasthenia gravis, Addison's disease, Sjögren's syndrome, pernicious anemia, celiac disease and vasculitis.

In some embodiments, the immune-mediated disease or condition is selected from the group consisting of eczema (atopic dermatitis), asthma, and a combination thereof. In some embodiments, the immune-mediated disease or condition is selected from the group consisting of a food allergy and a combination thereof. In some embodiments, the immune-mediated disease or condition is transplant rejection. In some embodiments, the immune-mediated disease or condition is a lysosomal storage disease.

In some embodiments, the exogenously modified population of monocytes do not present the autoantigenic peptide at the time of administration. In some embodiments, the administering comprises intravenous administration. In some embodiments, the administration of the pharmaceutical composition elicits an innate immune response in the subject.

In some embodiments, the monocytes of the exogenously modified population of monocytes migrate to a splenic marginal zone sinus of the subject after administration of the pharmaceutical composition. In some embodiments, the antigen presenting cells (APCs) of the subject engulf or phagocytose monocytes of the exogenously modified population of monocytes after administration of the pharmaceutical composition. In some embodiments, the monocytes of the exogenously modified population of monocytes undergo scavenger receptor-mediated uptake by APCs of the subject after administration of the pharmaceutical composition. In some embodiments, the APCs of the subject present the autoantigenic peptide in complex with an MHC protein after administration of the pharmaceutical composition.

In some embodiments, the MHC protein is an MHC class II protein. In some embodiments, the APCs of the subject secrete a cytokine or growth factor after administration of the pharmaceutical composition. In some embodiments, the cytokine or the growth factor regulates co-stimulatory molecules. In some embodiments, the cytokine or the growth factor is IL-10 and/or TGFbeta.

In some embodiments, the APCs of the subject increase expression a negative costimulatory molecule after administration of the pharmaceutical composition. In some embodiments, the negative costimulatory molecule comprises PD-L1, CTLA-4 or any combination thereof. In some embodiments, the APCs of the subject express IL-10 receptor. In some embodiments, the APCs of the subject are selected from the group consisting of B cells, macrophages, dendritic cells and a combination thereof. In some embodiments, the macrophages are marginal zone macrophages (MZMs).

In some embodiments, the effector T cells comprising a T cell receptor (TCR) specific to a peptide:MHC complex comprising the autoantigenic peptide undergo apoptosis after administration of the pharmaceutical composition. In some embodiments, the effector T cells comprising a TCR specific to a peptide:MHC complex comprising the autoantigenic peptide differentiate into anergic T cells after administration of the pharmaceutical composition. In some embodiments, the regulatory T cells (Tregs) of the subject comprising a TCR specific to a peptide:MHC complex comprising the autoantigenic peptide are upregulated after administration of the pharmaceutical composition. In some embodiments, the naive T cells of the subject differentiate into Tregs after administration of the pharmaceutical composition. In some embodiments, the naive T cells of the subject comprising a TCR specific to a peptide: MHC complex comprising the autoantigenic peptide differentiate into anergic T cells after administration of the pharmaceutical composition. In some embodiments, the T cells are CD4+ T cells.

In some embodiments, the exogenously modified population of monocytes comprises exogenously modified monocytes that are phagocytosed, engulfed and/or recognized as apoptotic by APCs of the subject.

In some embodiments, the method further comprises modifying a population of monocytes, thereby generating the exogenously modified population of monocytes. In some embodiments, the wherein modifying comprises introducing the recombinant polynucleotide into the population of monocytes. In some embodiments, introducing comprises electroporating, transfecting, nucleofecting or transducing. The method of any one of claims Error! Reference source not found.-Error! Reference source not found., wherein modifying comprises culturing the population of monocytes ex vivo. In some embodiments, culturing comprises inducing maturation, differentiation or apoptosis of the population of monocytes.

In some embodiments, the population of monocytes is an allogeneic or autologous population of monocytes. In some embodiments, modifying comprises contacting a population of monocytes with an agent that modifies the monocytes such that APCs of the human subject phagocytose or engulf exogenously modified monocytes of the exogenously modified population of monocytes. In some embodiments, the method comprises obtaining, isolating or enriching a population of monocytes from a biological sample from a human subject. In some embodiments, the biological sample is a peripheral blood mononuclear cell (PBMC) sample or a leukapheresis. In some embodiments, the human subject has T cells with a TCR that is specific to a peptide:MHC complex comprising the autoantigenic peptide.

In some embodiments, the method further comprises administering an additional therapeutic to the subject, wherein the additional therapeutic comprises an immunosuppressive agent. In some embodiments, the pharmaceutical composition is administered more than once. In some embodiments, the pharmaceutical composition is administered periodically at an interval of 2 months, 3 months, 4 months, 5 months, 6 months, 8 months, 10 months or 12 months.

In some embodiments, the human subject is positive for an HLA allele that specifically binds to the autoantigenic peptide. In some embodiments, the method comprises prior to administering the pharmaceutical composition, selecting a human subject that is positive for an HLA allele that specifically binds to the autoantigenic peptide.

In some embodiments, the immune-mediated disease or condition is selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo and Vogt-Koyanagi-Harada Disease.

In some embodiments, the recombinant polynucleotide is an RNA.

In some embodiments, the agent and the recombinant polynucleotide are encapsulated within a particle.

In some embodiments, the agent is specific for monocytes. In some embodiments, the agent does not induce apoptosis of the APCs that phagocytose, engulf and/or recognize the monocytes. In some embodiments, the agent is small molecule or gene. In some embodiments, the agent induces apoptosis of the monocytes of the human subject over time. In some embodiments, the agent is destroyed or rendered non-functional or degraded after the APCs phagocytose, engulf and/or recognize the monocytes. In some embodiments, the pharmaceutically acceptable excipient, diluent or carrier comprises a particle. In some embodiments, the pharmaceutically acceptable excipient, diluent or carrier comprises a lipid nanoparticle.

Provided herein is a method of treating or inducing immune tolerance to an autoantigenic peptide in a human subject with an immune-mediated disease or condition, the method comprising administering to the human subject the pharmaceutical composition comprising a recombinant polynucleotide as described anywhere in this disclosure. In some embodiments, the recombinant polynucleotide is associated with one or more lipid components. In some embodiments, the recombinant polynucleotide is encapsulated in a lipid nanoparticle.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 depicts a diagrammatic representation of an exemplary DNA plasmid vector schematically displaying the vector components, including coding regions for the polypeptide encoded by the vector, and briefly describes the steps useful to generate a tolerance inducing peptide. An exemplary therapeutic composition for use in treating a subject having an autoimmune or autoinflammatory disease may comprise such an exemplary vector or an exemplary mRNA or any intermediate form as depicted in the figure, as a tolerogenic composition. In some cases, such a vector or mRNA may be comprised in or assembled within a delivery vehicle, for example, a lipid nanoparticle (LNP).

FIG. 2A represents a prophetic data showing effect of the tolerogenic composition on the disease score in a prospective experimental study of an animal model of multiple sclerosis (MS or EAE). Tolerogenic modules are encoded by mRNA delivered in a lipid nanoparticle delivery vehicle, as depicted graphically in the diagram (right). The study will include three groups of mice, one group of mice will receive a vehicle control of the tolerance module composition; one group of mice will receive the mRNA-LNP encoding tolerogenic peptide at the time of immunization (Day 0); and one group will receive the mRNA-LNP encoding tolerogenic peptide at peak inflammation period after immunization, e.g., day 17-19. (Day 19). Disease score represents the severity of the disease on a relative scale of 0-4, 4 being most severe. The determination is based on observed symptoms of MS related illness.

FIG. 2B represents a prophetic data showing effect of a tolerogenic composition in a prophetic experimental study on an animal model of multiple sclerosis (MS) comparing a number of exemplary tolerogenic modules. Exemplary mRNA modules for tolerance encode for proteolipid protein (PLP)-TGF-beta (PLP-TGFB), PLP-(Programed Death Ligand 1 (PDL1)-TGFB, PLP-PDL1-TGFB-IL10 and/or PLP-PDL1. Mean clinical score represents the severity of the disease on a relative scale of 0-4, 4 being most severe. The determination is based on observed symptoms of MS related illness.

FIG. 3 depicts a schematic showing an exemplary process for generating the pharmaceutical cellular product (a tolerogenic cellular composition) comprising myeloid cells expressing a tolerogenic agent, such as a protein, or peptide. Briefly, the process involves generation and amplification of plasmid comprising the recombinant nucleic acid is followed by generating transcripts in vitro, which is then purified. CD14+ cells are isolated from a biological sample previously stored or obtained directly from a subject (e.g., leukapheresis sample), and the mRNA is electroporated to the cells. Cells are washed, an apoptosis inducing agent is administered, then washed and recovered and stored in liquid nitrogen, and thawed when needed for administration.

FIG. 4 depicts an exemplary schematic diagram indicating a process of generating a tolerogenic cellular product as a cellular therapeutic composition to treat an autoimmune or autoinflammatory disease. Top diagram shows an exemplary recombinant mRNA encoding one or more tolerogenic components such as an antigen, and LAMP1; a downward arrow indicate a simplified process of expressing the exemplifying mRNA in a cell, e.g., via electroporating human cells for example, CD14+(myeloid) cells isolated from a leukopak sample (bottom diagrams), to express a gene (e.g., antigen, or fusion protein of interest), treating with an agent, for example ECDI for inducing apoptosis, followed by infusion.

FIG. 5A is brief schematic showing a workflow from isolation to administration of a tolerogenic cell therapy product as described herein, which takes between 1-3 days.

FIG. 5B is a schematic diagram of an engineered tolerogenic myeloid cell expressing a gene of interest, e.g., a recombinant DNA or RNA incorporated in the myeloid cell, wherein the recombinant DNA or RNA encodes an antigen, wherein the myeloid cell presents the antigen in an MHC-dependent manner for tolerance induction in vivo.

FIG. 5C is an exemplary flow cytometry data showing expression of antigens encoded by the recombinant polynucleotide incorporated in the cell.

FIG. 6 is an exemplary figure from a prophetic example, showing disease score from a mouse model of MS, induced by subcutaneous injection of Proteolipid peptide (aa139-151) (PLP), treated with either tolerogenic myeloid cells expressing PLP (MYE-PLP) or myeloid cells expressing OVA peptide (MYE-OVA, as negative control) or vehicle.

FIG. 7A EAE (mouse model of MS) was induced by subcutaneous injection of Proteolipid peptide (PLP, aa139-151) emulsified in CFA ion 30 mice. Mice were then randomized into 3 groups of ten mice. Group one received no treatment, Group 2 received 1×10⁷ MIT₁₃₉₋₁₅₁ cells at the time of immunization and Group 3 received 1×10⁷ MIT₁₃₉₋₁₅₁ cells at peak disease (day 17). Treatment with MIT cells results in prevention of disease (group 2 animals). In animals with ongoing disease, treatment resulted in amelioration of disease and the prevention of relapse. Together the data show MIT cells induce immune tolerance.

FIG. 7B. Splenic cells from the mice in FIG. 7A, expressing IFN-gamma (pg/ml). IFN-gamma from mock cells (green circles), non-transfected treated cells (NT treated control, triangles), and MT cells expressing the antigen and treated. Splenic cells from mice treated with MIT cells modified ex vivo that express PLP have statistically significant reduction in IFN response compared to non-transfected cells.

FIG. 8A is a schematic showing product manufacturing process for generating tolerogenic myeloid cells (MIT) expressing exemplary antigens, such as Gliadin epitopes is shown, for generating an exemplary composition for treating, for example, celiac disease.

FIG. 8B is a schematic showing product testing protocol for generating tolerogenic myeloid cells expressing exemplary antigens, such as Gliadin epitopes. The mice from each group would be monitored for body weight. Cells isolated from the animals are used for Gliadin ex vivo recall responses, and sacrificed for tissue collection and histology.

FIG. 8C shows a prophetic data from monitoring body weight from mice treated with gliadin epitope expressing myeloid cells (MYE-Gliadin), OVA-expressing myeloid cells (MYE-OVA) a positive control of mice fed with gluten free diet and a negative control of mice fed with gluten diet.

FIG. 8D shows a prophetic data of summarizing histological findings on duodenitis in mice as a result of allergic reaction to gluten in diet. Each data point represent a duodenitis score (set in an arbitrary scale of 0-10) from an experimental mouse.

FIG. 9 shows the various modifications included in the designs of the mRNA.

FIG. 10 shows diagrammatic representation of a simplified plasmid map for expressing an inserted gene in a myeloid cell.

FIG. 11 shows a schematic diagram of an mRNA comprising sequences encoding immune regulatory signals in tandem two or more of TGF-beta, PDL1, IL10 etc., and the autoimmune antigen.

FIG. 12A (top panel) shows a schematic diagram an mRNA comprising sequences encoding tolerogenic modules for therapy in an experimental liver fibrosis model, e.g., a sequence encoding MMP12, anti-HT-2BR scFV and anti-TGFB scFV, separated by auto-cleavable sequences (e.g., T2A) and IRES. Bottom panel, an experimental animal disease model is exemplified herein, showing schematic workflow of the administration of various components to induce liver fibrosis and test the tolerogenic myeloid cell therapeutic product described herein. CCL4 is repeatedly injected to the experimental animal at indicated period to induce fibrosis. Engineered myeloid cells expressing the mRNA encoding the tolerogenic module is administered at the days indicated in the schematic workflow diagram.

FIG. 12B shows evaluation of the effect of the treatments administered in FIG. 12A. In this prophetic example, liver function enzymes ALT and AST are tested at the indicated periods.

FIG. 13 (top panel) shows a schematic diagram a recombinant mRNA comprising sequences encoding tolerogenic modules for therapy in an experimental graft versus host disease (GVHD) mice model, e.g., Bottom panel, prophetic data showing an experimental animal disease model of GVHD, and that treatment of the mice with experimental GVHD with the therapeutic myeloid cells expressing recombinant mRNA reduces mortality from the disease.

### DETAILED DESCRIPTION

The diverse functionality of myeloid cells makes them an ideal cell therapy candidate that can be engineered to have numerous therapeutic effects. The present disclosure is related to immunotherapy using myeloid cells (e.g., CD14+ cells) of the immune system, particularly antigen presenting cells (APCs). A number of therapeutic indications could be contemplated using myeloid cells. For example, myeloid cell immunotherapy could be exceedingly important in cancer, autoimmunity, fibrotic diseases and infections. The present disclosure is related to immunotherapy using myeloid cells, including APCs e.g., macrophages, that are modified ex vivo. It is an object of the invention disclosed herein to harness one or more of these functions of myeloid cells for therapeutic uses. For example, it is an object of the invention disclosed herein to harness the antigen presenting activity of myeloid cells, including engineered myeloid cells, for therapeutic uses. For example, it is an object of the invention disclosed herein to harness the ability of myeloid cells, including engineered myeloid cells that have been modified ex vivo, to induce antigen specific tolerance of T cells. For example, it is an object of the invention disclosed herein to harness the ability of myeloid cells, including engineered myeloid cells, to promote activation of tolerogenic APCs, e.g., dendritic cells (DCs). For example, it is an object of the invention disclosed herein to harness the ability of myeloid cells, including engineered myeloid cells, to reduce recruitment and trafficking of immune cells and molecules.

In some embodiments, the present disclosure involves making and using engineered myeloid cells (e.g., CD14+ cells, such as macrophages or other APCs, which can be introduced into a tissue to induce antigen-specific tolerance. Engineered myeloid cells, such as macrophages and other phagocytic cells, can be prepared by incorporating nucleic acid sequences (e.g., mRNA, plasmids, viral constructs) encoding a chimeric fusion protein (CFP), that has an extracellular binding domain specific to disease associated antigens (e.g., autoimmune antigens), into the cells using, for example, recombinant nucleic acid technology, synthetic nucleic acids, gene editing techniques (e.g., CRISPR), transduction (e.g., using viral constructs), electroporation, or nucleofection. It has been found that myeloid cells can be engineered to have a broad and diverse range of activities. For example, it has been found that myeloid cells can be engineered to express a recombinant polynucleotide encoding one or more antigens to have a broad and diverse range of activities. For example, it has been found that myeloid cells can be engineered to harbor a recombinant nucleic acid that encodes one or more antigens, e.g., autoimmune antigens, such that upon introduction into the body of a subject, the myeloid cells induce tolerogenic response against the one or more antigens in the subject, where the subject had exhibited an increased immunogenic response to at least one of the one or more antigen prior to the introducing the engineered myeloid cells. In some embodiments, the myeloid cells can be engineered to promote secretion of tolerogenic molecules such that upon introducing the engineered cells in a subject exhibiting a pathologically increased immune activation prior to the administering of the engineered myeloid cells, and whereupon introducing the engineered cells in the subject, reduces or ameliorates the pathologically increased immune activation. A pathologically increased immune response as used herein can be described as an undesired immune response against a self-antigen, e.g., an autoantigen; or against a non-self-antigen, such as in a grafted tissue in a graft versus host immune response, or such as in a host versus graft immune response; or can be described as an undesired and/or uncontrolled immune response, such as for example, an allergic response, a hyperactive immune response e.g., cytokine storm, or an immune sequelae against a foreign antigen. A person of skill in the art can envisage situations encompassed broadly as a pathologically increased immune response, even if not articulated herein. In some embodiments, the engineered myeloid cells promote secretion of tolerogenic molecules in an inflamed or allergic tissue of a subject. The engineered myeloid cells can be engineered to suppress or reduce recruitment and trafficking of immune cells and molecules responsive to an antigen to a tissue exhibiting an aberrant immune activation or aberrant immune response. An aberrant immune response as described herein can be described as an undesired immune response against a self-antigen, e.g., an autoantigen; or against a non-self-antigen, such as in a grafted tissue in a graft versus host immune response, or such as in a host versus graft immune response; or can be described as an undesired and/or uncontrolled immune response, such as for example, an allergic response, a hyperactive immune response e.g., cytokine storm, or an immune sequelae against a foreign antigen. A person of skill in the art can envisage situations encompassed broadly as a pathologically increased immune response, even if not articulated herein.

Engineered myeloid cells can also be short-lived in vivo, phenotypically diverse, sensitive, plastic, and are often found to be difficult to manipulate in vitro. For example, exogenous gene expression in monocytes has been difficult compared to exogenous gene expression in non-hematopoietic cells. There are significant technical difficulties associated with transfecting myeloid cells (e.g., monocytes/macrophages). As professional phagocytes, myeloid cells, such as monocytes/macrophages, comprise many potent degradative enzymes that can disrupt nucleic acid integrity and make gene transfer into these cells an inefficient process. This is especially true of activated macrophages which undergo a dramatic change in their physiology following exposure to immune or inflammatory stimuli. Viral transduction of these cells has been hampered because macrophages are end-stage cells that generally do not divide; therefore, some of the vectors that depend on integration into a replicative genome have met with limited success. The present disclosure provides innovative methods and compositions that can successfully transfect or transduce a myeloid cell, or otherwise induce a genetic modification in a myeloid cell, with the purpose of augmenting a functional aspect of a myeloid cell, additionally, without compromising the cell's differentiation capability, maturation potential, and/or its plasticity. In some embodiments, myeloid cells are manipulated ex vivo, such that upon introducing into a subject. the manipulated myeloid cells comprising the genetic modification are readily taken up by active phagocytes in vivo, which then process antigens comprised in the manipulated engineered myeloid cells, e.g., the one or more antigens encoded by the recombinant polynucleotide in the engineered myeloid cells, as described in the previous paragraphs, and display on the membrane surface, and induce tolerance against the antigens in vivo.

All terms are intended to be understood as they would be understood by a person skilled in the art. Unless defined otherwise, all technical and scientific terms used herein have the same meaning as commonly understood by one of ordinary skill in the art to which the disclosure pertains. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment. As used herein, the singular forms “a,” “an,” and “the” include the plural referents unless the context clearly indicates otherwise. The terms “include,” “such as,” and the like are intended to convey inclusion without limitation, unless otherwise specifically indicated.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

The term “about” or “approximately” as used herein when referring to a measurable value such as a parameter, an amount, a temporal duration, and the like, is meant to encompass variations of +/−30% or less, +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

An “agent” can refer to any cell, small molecule chemical compound, antibody or fragment thereof, nucleic acid molecule, or polypeptide.

An “alteration” or “change” can refer to an increase or decrease. For example, an alteration can be an increase or decrease of 1%, 2%, 3%, 4%, 5%, 10%, 20%, 30%, or by 40%, 50%, 60%, or even by as much as 70%, 75%, 80%, 90%, or 100%. For example, an alteration can be an increase or decrease of 1-fold, 2-fold, 3-fold, 4-fold, 5-fold, 10-fold, 20-fold, 30-fold, or by 40-fold, 50-fold, 60-fold, or even by as much as 70-fold, 75-fold, 80-fold, 90-fold, or 100-fold.

An “antigen presenting cell” or “APC” as used herein includes professional antigen presenting cells (e.g., B lymphocytes, macrophages, monocytes, dendritic cells, Langerhans cells), as well as other antigen presenting cells (e.g., keratinocytes, endothelial cells, astrocytes, fibroblasts, oligodendrocytes, thymic epithelial cells, thyroid epithelial cells, glial cells (brain), pancreatic beta cells, and vascular endothelial cells). An APC can express Major Histocompatibility complex (MHC) molecules and can display antigens complexed with MHC on its surface which can be recognized by T cells and trigger T cell activation and an immune response. Professional antigen-presenting cells, notably dendritic cells, play a key role in stimulating naive T cells. Nonprofessional antigen-presenting cells, such as fibroblasts, may also contribute to this process. APCs can also cross-present peptide antigens by processing exogenous antigens and presenting the processed antigens on class I MHC molecules. Antigens that give rise to proteins that are recognized in association with class I MHC molecules are generally proteins that are produced within the cells, and these antigens are processed and associate with class I MHC molecules.

As used herein, the term “anergy,” “tolerance,” or “antigen-specific tolerance” refers to insensitivity of T cells to T cell receptor-mediated stimulation. Such insensitivity is generally antigen-specific and persists after exposure to the antigenic peptide has ceased. For example, anergy in T cells is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP 1 sequence that can be found within the enhancer (Kang et al. 1992 Science. 257: 1134).

The term “antigen enhancer” refers to a non-specific immune response enhancer with which the antigen is mixed or incorporated.

The term “antibody” refers to a class of proteins that are generally known as immunoglobulins, including, but not limited to IgG1, IgG2, IgG3, and IgG4), IgA (including IgA1 and IgA2), IgD, IgE, IgM, and IgY, The term “antibody” includes, but is not limited to, full length antibodies, single-chain antibodies, single domain antibodies (sdAb) and antigen-binding fragments thereof. Antigen-binding antibody fragments include, but are not limited to, Fab, Fab′ and F(ab′)2, Fd (consisting of V_{H} and C_{H}1), single-chain variable fragment (scFv), single-chain antibodies, disulfide-linked variable fragment (dsFv) and fragments comprising a V_{L} and/or a V_{H} domain. Antibodies can be from any animal origin. Antigen-binding antibody fragments, including single-chain antibodies, can comprise variable region(s) alone or in combination with tone or more of a hinge region, a CH1 domain, a CH2 domain, and a CH3 domain. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex.

An “antigen (Ag)” refers to a compound, composition, or substance that can stimulate the production of antibodies or a T cell response in an animal.

A “biological sample” can refer to any tissue, cell, fluid, or other material derived from an organism.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

The “control elements” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used. In particular embodiments, a vector for use in practicing the invention including, but not limited to expression vectors and viral vectors, will include exogenous, endogenous, or heterologous control sequences such as promoters and/or enhancers.

The term “constitutive gene” as used herein refers to a gene that is transcribed continually as opposed to a facultative gene, which is only transcribed when needed. The term “highly expressed gene” as used herein refers to a gene that is expressed broadly in diverse cell types, or at higher levels than other genes.

The terms “complement,” “complements,” “complementary,” and “complementarity,” as used herein, refer to a sequence that is complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/embossneedle/nucleotide.html), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.ukaools/psa/emboss_water/nucleotide.html, optionally with default settings). Optimal alignment can be assessed using any suitable parameters of a chosen algorithm, including default parameters.

Complementarity may be perfect or substantial/sufficient. Perfect complementarity between two nucleic acids may mean that the two nucleic acids may form a duplex in which every base in the duplex is bonded to a complementary base by Watson-Crick pairing. Substantial or sufficient complementary may mean that, a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions may be predicted by using the sequences and standard mathematical calculations to predict the melting temperature (Tₘ) of hybridized strands, or by empirical determination of Tₘ by using routine methods.

Described herein are compositions and methods for the design, preparation, manufacture and/or formulation of circular polynucleotides including circular RNA. As used herein, “circular RNA” or “circRNA” means a single stranded circular polynucleotide which acts substantially like, and has the properties of, an RNA, and that can encode at least one polypeptide of interest. The term “circular” is also meant to encompass any secondary or tertiary configuration of the circular RNA.

The term “elutriation” refers to a purification, separation, or removal process that separates cells based on differences in their density. Preferably, the elutriation process to purify human monocytes is centrifugal elutriation.

The term “epitope” includes any protein determinant capable of specific binding to an antibody, antibody peptide, and/or antibody-like molecule (including but not limited to a T cell receptor) as defined herein. Epitopic determinants typically consist of chemically active surface groups of molecules such as amino acids or sugar side chains and generally have specific three-dimensional structural characteristics as well as specific charge characteristics.

The term “electroporation” refers to application of an electric voltage to cells in the presence of a material desired to be delivered inside cells, temporarily allowing cell membranes to become porous to passage of materials, such as nucleic acids, into the cells. Conditions used for electroporation include selection of voltage used, pulse width and number of pulses. Typically, a voltage in the range of about 800 V/cm-1400 V/cm is applied in a pulse of about 8 milliseconds-15 milliseconds. More than one pulse can be applied, typically 1-3 pulses are applied. Particular conditions selected depend on variables such as cell type, size and species from which the cell is derived and such conditions are selected by one of skill in the art. Electroporation methods are well-known in the art, for example, as described in J. A. Nickoloff, Animal Cell Electroporation and Electrofusion Protocols, Humana Press; 1st ed., 1995.

The term “engineered” refers to cells that engineered by genetic engineering means. Engineered cells are modified as compared to naturally occurring cells. For example, an engineered monocyte generated according to the present method carries a nucleic acid comprising a nucleotide sequence that does not naturally occur in the monocytes from which it is derived.

The term “fusion polypeptide” is known to the skilled person and relates to a polypeptide comprising at least two polypeptides, e.g. proteins, protein domains, or parts thereof, linked covalently, preferably by a peptide bond. A chimeric fusion protein as used herein can mean a fusion polypeptide (e.g., a fusion protein), where one or more isolated components from two or more heterogenous proteins or polypeptides are fused to form the fusion polypeptide. In some embodiments a chimeric fusion polypeptide may designate a recombinant chimeric polypeptide comprising, for example, two or more antigens from two or more different antigenic proteins. In some embodiments, a chimeric fusion protein is a engineered to be expressed on the surface of the cell. In some embodiments, a chimeric fusion protein may be a recombinant receptor protein. In some embodiments, the chimeric fusion protein may comprise an antigen, wherein the antigen is presented by an antigen presenting cell expressing the recombinant polynucleotide encoding the chimeric fusion protein.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

A “fragment” can refer to a portion of a protein or nucleic acid. In some embodiments, a fragment retains at least 50%, 75%, or 80%, or 90%, 95%, or even 99% of the biological activity of a reference protein or nucleic acid.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO: 6), where R is a purine (A or G) (Kozak, 1986. Cell. 44(2):283-92, and Kozak, 1987. Nucleic Acids Res. 15(20):8125-48)

An engineered cell, such as an engineered myeloid cell, can refer to a cell that has at least one exogenous nucleic acid sequence in the cell, even if transiently expressed. Expressing an exogenous nucleic acid may be performed by various methods described elsewhere, and encompasses methods known in the art. The present disclosure relates to preparing and using engineered cells, for example, engineered myeloid cells, such as engineered phagocytic cells. The present disclosure relates to, inter alia, an engineered cell comprising an exogenous nucleic acid encoding, for example, a chimeric fusion protein (CFP).

The term “exon” refers to a nucleic acid sequence found in genomic DNA that is bioinformatically predicted and/or experimentally confirmed to contribute contiguous sequence to a mature mRNA transcript. The term “intron” refers to a sequence present in genomic DNA that is bioinformatically predicted and/or experimentally confirmed to not encode part of or all of an expressed protein, and which, in endogenous conditions, is transcribed into RNA (e.g. pre-mRNA) molecules, but which is spliced out of the endogenous RNA (e.g. the pre-mRNA) before the RNA is translated into a protein.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al, 1990. Trends Biochem Sci 15(12):477-83) and Jackson and Kaminski. 1995. RNA 1(10):985-1000. An “internal ribosome entry site” or “IRES” refers to a nucleotide sequence that allows for 5′-end/cap-independent initiation of translation and thereby raises the possibility to express 2 proteins from a single messenger RNA (mRNA) molecule. IRESs are commonly located in the 5′ UTR of positive-stranded RNA viruses with uncapped genomes. Another means to express 2 proteins from a single mRNA molecule is by insertion of a 2A peptide(-like) sequence in between their coding sequence. 2A peptide(-like) sequences mediate self-processing of primary translation products by a process variously referred to as “ribosome skipping”, “stop-go” translation and “stop carry-on” translation. 2A peptide(-like) sequences are present in various groups of positive- and double-stranded RNA viruses including Picornaviridae, Flaviviridae, Tetraviridae, Dicistroviridae, Reoviridae and Totiviridae.

The term “2A peptide” refers to a class of 18-22 amino-acid (AA)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” is believed to be ribosome skipping the formation of a glycyl-prolyl peptide bond at the C-terminus of the 2A sequence. 2A peptide(-like) sequences mediate self-processing of primary translation products by a process variously referred to as “ribosome skipping”, “stop-go” translation and “stop carry-on” translation. 2A peptide(-like) sequences are present in various groups of positive- and double-stranded RNA viruses including Picornaviridae, Flaviviridae, Tetraviridae, Dicistroviridae, Reoviridae and Totiviridae.

As used herein, the term “operably linked” refers to a functional relationship between two or more segments, such as nucleic acid segments or polypeptide segments. Typically, it refers to the functional relationship of a transcriptional regulatory sequence to a transcribed sequence.

The term “termination sequence” refers to a nucleic acid sequence which is recognized by the polymerase of a host cell and results in the termination of transcription. The termination sequence is a sequence of DNA that, at the 3′ end of a natural or synthetic gene, provides for termination of mRNA transcription or both mRNA transcription and ribosomal translation of an upstream open reading frame. Prokaryotic termination sequences commonly comprise a GC-rich region that has a two-fold symmetry followed by an AT-rich sequence. A commonly used termination sequence is the T7 termination sequence. A variety of termination sequences are known in the art and may be employed in the nucleic acid constructs of the present invention, including the TINT3, TL13, TL2, TR1, TR2, and T6S termination signals derived from the bacteriophage lambda, and termination signals derived from bacterial genes, such as the trp gene of E. coli.

The term “immune response” includes, but is not limited to, T cell mediated, NK cell mediated and/or B cell mediated immune responses. These responses may be influenced by modulation of T cell costimulation and NK cell costimulation. Exemplary immune responses include T cell responses, e.g., cytokine production, and cellular cytotoxicity. In addition, immune responses include immune responses that are indirectly affected by NK cell activation, B cell activation and/or T cell activation, e.g., antibody production (humoral responses) and activation of cytokine responsive cells, e.g., macrophages. Immune responses include adaptive immune responses. The adaptive immune system can react to foreign molecular structures, such as antigens of an intruding organism. Unlike the innate immune system, the adaptive immune system is highly specific to a pathogen. Adaptive immunity can also provide long-lasting protection. Adaptive immune reactions include humoral immune reactions and cell-mediated immune reactions. In humoral immune reactions, antibodies secreted by B cells into bodily fluids bind to pathogen-derived antigens leading to elimination of the pathogen through a variety of mechanisms, e.g. complement-mediated lysis. In cell-mediated immune reactions, T cells capable of destroying other cells are activated. For example, if proteins associated with a disease are present in a cell, they can be fragmented proteolytically to peptides within the cell. Specific cell proteins can then attach themselves to the antigen or a peptide formed in this manner, and transport them to the surface of the cell, where they can be presented to molecular defense mechanisms, such as T cells. Cytotoxic T cells can recognize these antigens and kill cells that harbor these antigens.

The term “major histocompatibility complex (MHC)”, “MHC molecule”, or “MHC protein” refers to a protein capable of binding an antigenic peptide and present the antigenic peptide to T lymphocytes. Such antigenic peptides can represent T cell epitopes. The human MHC is also called the HLA complex. Thus, the terms “human leukocyte antigen (HLA)”, “HLA molecule” or “HLA protein” are used interchangeably with the terms “major histocompatibility complex (MHC)”, “MHC molecule”, and “MHC protein”. HLA proteins can be classified as HLA class I or HLA class II. The structures of the proteins of the two HLA classes are very similar; however, they have very different functions. Class I HLA proteins are present on the surface of almost all cells of the body, including most tumor cells. Class I HLA proteins are loaded with antigens that usually originate from endogenous proteins or from pathogens present inside cells, and are then presented to naive or cytotoxic T-lymphocytes (CTLs). HLA class II proteins are present on antigen presenting cells (APCs), including but not limited to dendritic cells, B cells, and macrophages. They mainly present peptides which are processed from external antigen sources, e.g. outside of cells, to helper T cells.

In the HLA class II system, phagocytes such as macrophages and immature dendritic cells can take up entities by phagocytosis into phagosomes—though B cells exhibit the more general endocytosis into endosomes—which fuse with lysosomes whose acidic enzymes cleave the uptaken protein into many different peptides. Autophagy is another source of HLA class II peptides. The most studied subclass II HLA genes are: HLA-DPA1, HLA-DPB1, HLA-DQA1, HLA-DQB1, HLA-DRA, and HLA-DRB1.

Presentation of peptides by HLA class II molecules to CD4+ helper T cells can lead to immune responses to foreign antigens. Once activated, CD4+ T cells can promote B cell differentiation and antibody production, as well as CD8+ T cell (CTL) responses. CD4+ T cells can also secrete cytokines and chemokines that activate and induce differentiation of other immune cells. HLA class II molecules are typically heterodimers of α- and β-chains that interact to form a peptide-binding groove that is more open than class I peptide-binding grooves.

HLA alleles are typically expressed in codominant fashion. For example, each person carries 2 alleles of each of the 3 class I genes, (HLA-A, HLA-B and HLA-C) and so can express six different types of class II HLA. In the class II HLA locus, each person inherits a pair of HLA-DP genes (DPA1 and DPB1, which encode α and β chains), HLA-DQ (DQA1 and DQB1, for α and β chains), one gene HLA-DRa (DRA1), and one or more genes HLA-DRO (DRB1 and DRB3, -4 or -5). HLA-DRB1, for example, has more than nearly 400 known alleles. That means that one heterozygous individual can inherit six or eight functioning class II HLA alleles: three or more from each parent. Thus, the HLA genes are highly polymorphic; many different alleles exist in the different individuals inside a population. Genes encoding HLA proteins have many possible variations, allowing each person's immune system to react to a wide range of foreign invaders. Some HLA genes have hundreds of identified versions (alleles), each of which is given a particular number. In some embodiments, the class I HLA alleles are HLA-A*02:01, HLA-B*14:02, HLA-A*23:01, HLA-E*01:01 (non-classical). In some embodiments, class II HLA alleles are HLA-DRB*01:01, HLA-DRB*01:02, HLA-DRB*11:01, HLA-DRB*15:01, and HLA-DRB*07:01.

Nucleic acid molecules useful in the methods of the disclosure include, but are not limited to, any nucleic acid molecule with activity or that encodes a polypeptide. Polynucleotides having substantial identity to an endogenous sequence are typically capable of hybridizing with at least one strand of a double-stranded nucleic acid molecule. “Hybridize” refers to when nucleic acid molecules pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration can ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions can ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an exemplary embodiment, hybridization can occur at 30° C. in 750 mM NaCl, 75 mM trisodium citrate, and 1% SDS. In another exemplary embodiment, hybridization can occur at 37° C. in 500 mM NaCl, 50 mM trisodium citrate, 1% SDS, 35% formamide, and 100 μg/ml denatured salmon sperm DNA (ssDNA). In another exemplary embodiment, hybridization can occur at 42° C. in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, or less than about 15 mM NaCl and 1.5 mM trisodium citrate. Stringent temperature conditions for the wash steps can include a temperature of at least about 25° C., of at least about 42° C., or at least about 68° C. In exemplary embodiments, wash steps can occur at 25° C. in 30 mM NaCl, 3 mM trisodium citrate, and 0.1% SDS. In other exemplary embodiments, wash steps can occur at 42° C. in 15 mM NaCl, 1.5 mM trisodium citrate, and 0.1% SDS. In another exemplary embodiment, wash steps can occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

A tolerogenic module may refer to a composition that may comprise an mRNA, encoding a tolerance inducing agent, such as a protein or peptide, wherein the mRNA is encapsulated in a delivery vehicle, such as a lipid nanoparticle (LNP). The tolerance inducing protein or peptide may be a polypeptide. The tolerance inducing protein, peptide or polypeptide may be a cytokine or a chemokine, an immunosuppressant molecule, TGF-beta (or TGF-β), IL4, IL10, IL11, IL13, IL33, IL35, IL37 etc., or a checkpoint molecule such as PD1 or PDL1. The tolerogenic module may comprise proteolipid protein emulsified with an adjuvant. In some cases, the adjuvant may be Freund's adjuvant.

A clinical score, mean clinical score or a diseases score may refer to a form of measuring the disease severity, and may be based on a determination of a number of manifestations relevant to the disease it pertains to in a subject who is examined at a given time period. A clinical score, mean clinical score or a diseases score may be predetermined as a system of evaluation that would apply to all observations in a clinical study or may be used as a mode of determination of disease severity in general across a practice area in medicine. The manifestations referred to herein may be observable symptoms, observable physiological outcomes or features that result from a disease, (e.g., fever, swelling, pain) and may be used to determine the severity of the illness or sickness experienced by a subject at the time of observation. For example, the clinical score or a diseases score may be an arbitrary score as determined by a practitioner in the relevant field of medicine, where severity of one or more symptoms of the disease are indicated in a predetermined numerical scale and unit of preference, or on a relative numerical scale without an unit of measurement. For example, a symptomatic illness that comprises of a fever and an inflammation may each be allotted a scoring system in the preferred units and a clinical score may be thereafter determined that indicates the relative degree of illness based on the measurements of fever and inflammation separately, and accounted for jointly by the clinical score or disease score. For example, a mean clinical score may be provided in an arbitrary scale of 0 to 4, where 0 is the lack of any symptoms or manifestation and 4 is the highest score of the disease manifestation in the form of its symptoms.

“Phagocytosis” may be used interchangeably with “engulfment” and can refer to a process by which a cell engulfs a particle, such as a cancer cell or an infected cell. This process can give rise to an internal compartment (phagosome) containing the particle. This process can be used to ingest and or remove a particle, such as a cancer cell or an infected cell from the body.

A “polypeptide” can refer to a molecule containing amino acids linked together via a peptide bond, such as a glycoprotein, a lipoprotein, a cellular protein or a membrane protein. A polypeptide may comprise one or more subunits of a protein. A polypeptide may be encoded by a recombinant nucleic acid. In some embodiments, polypeptide may comprise more than one peptide sequence in a single amino acid chain, which may be separated by a spacer, a linker or peptide cleavage sequence. A polypeptide may be a fused polypeptide. A polypeptide may comprise one or more domains, modules or moieties. In some cases, a polypeptide may be used interchangeably with the term “protein”.

The expression “pharmaceutically acceptable” may refer to the ingredients of a pharmaceutical composition are compatible with each other and not deleterious to the subject to which it is administered. The expression “pharmaceutically acceptable carrier, diluent, excipient and/or adjuvant” refers to a substance that does not produce an adverse, allergic or other untoward reaction when administered to an animal, preferably a human. It includes any and all inactive substances such as for example solvents, cosolvents, antioxidants, surfactants, stabilizing agents, emulsifying agents, buffering agents, pH modifying agents, preserving agents (or preservating agents), antibacterial and antifungal agents, isotonifiers, granulating agents or binders, lubricants, disintegrants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g., FDA Office or EMA.

The term “polyA site” or “polyA sequence” as used herein may denote a DNA sequence which directs both the termination and polyadenylation of the nascent RNA transcript by RNA polymerase II. A poly A sequence may refer to the terminal nucleotides denoted by adenosine repeats in an mRNA. The terms “polyadenylation sequence” (also referred to as a “poly A site” or “poly A sequence”) refers to a DNA sequence which directs both the termination and polyadenylation of the nascent RNA transcript. Polyadenylation sequences can promote mRNA stability by addition of a polyA tail to the 3′ end of the coding sequence and thus, contribute to increased translational efficiency. Efficient polyadenylation of the recombinant transcript is desirable as transcripts lacking a poly A tail are unstable and are rapidly degraded. Illustrative examples of polyA signals that can be used in a vector of the invention, includes an ideal polyA sequence (e.g., AATAAA, ATT AAA, AGTAAA), a bovine growth hormone polyA sequence (BGHpA), a rabbit β-globin polyA sequence (rPgpA), or another suitable heterologous or endogenous polyA sequence known in the art. Efficient polyadenylation of the recombinant transcript is desirable, as transcripts lacking a poly A tail are typically unstable and rapidly degraded. The poly A signal utilized in an expression vector may be “heterologous” or “endogenous”. An endogenous poly A signal is one that is found naturally at the 3′ end of the coding region of a given gene in the genome. A heterologous poly A signal is one which is isolated from one gene and placed 3′ of another gene, e.g., coding sequence for a protein. A commonly used heterologous poly A signal is the SV40 poly A signal. The SV40 poly A signal is contained on a 237 bp BamHI/BclI restriction fragment and directs both termination and polyadenylation; numerous vectors contain the SV40 poly A signal. Another commonly used heterologous poly A signal is derived from the bovine growth hormone (BGH) gene; the BGH poly A signal is also available on a number of commercially available vectors. The poly A signal from the Herpes simplex virus thymidine kinase (HSV tk) gene is also used as a poly A signal on a number of commercial expression vectors. The polyadenylation signal facilitates the transportation of the RNA from within the cell nucleus into the cytosol as well as increases cellular half-life of such an RNA. The polyadenylation signal is present at the 3′-end of an mRNA.

The term “recombinant nucleic acid” may refer to a nucleic acid prepared, expressed, created or isolated by recombinant means. A recombinant nucleic acid can contain a nucleotide sequence that is not naturally occurring. The term “recombinant nucleic acid” may be interchangeably used with “recombinant polynucleotide” throughout the document, and is understood in this context to mean the same. A recombinant nucleic acid may be synthesized in the laboratory. A recombinant nucleic acid may be prepared by using recombinant DNA technology, for example, enzymatic modification of DNA, such as enzymatic restriction digestion, ligation, and DNA cloning. A recombinant nucleic acid can be DNA, RNA, analogues thereof, or a combination thereof. A recombinant DNA may be transcribed ex vivo or in vitro, such as to generate a messenger RNA (mRNA). A recombinant mRNA may be isolated, purified and used to transfect a cell. A recombinant nucleic acid may encode a protein or a polypeptide. The process of introducing or incorporating a nucleic acid into a cell can be via transformation, transfection or transduction. Transformation is the process of uptake of foreign nucleic acid by a bacterial cell. This process is adapted for propagation of plasmid DNA, protein production, and other applications. Transformation introduces recombinant plasmid DNA into competent bacterial cells that take up extracellular DNA from the environment. Some bacterial species are naturally competent under certain environmental conditions, but competence is artificially induced in a laboratory setting. Transfection is the introduction of small molecules such as DNA, RNA, or antibodies into eukaryotic cells. Transfection may also refer to the introduction of bacteriophage into bacterial cells. ‘Transduction’ is mostly used to describe the introduction of recombinant viral vector particles into target cells, while ‘infection’ refers to natural infections of humans or animals with wild-type viruses.

“Substantially identical” may refer to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence can be at least 60%, 80% or 85%, 90%, 95%, 96%, 97%, 98%, or even 99% or more identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-m° indicating a closely related sequence. A “reference” is a standard of comparison. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment to a reference sequence and determination of homologous residues.

The terms “spacer” or “linker” as used in reference to a fusion protein may refer to a peptide sequence that joins two other peptide sequences of the fusion protein. In some embodiments, a linker or spacer has no specific biological activity other than to join or to preserve some minimum distance or other spatial relationship between the proteins or RNA sequences. In some embodiments, the constituent amino acids of a spacer can be selected to influence some property of the molecule such as the folding, flexibility, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. In some embodiments, a linker is used to separate two or more polypeptides, e.g. two antigenic peptides by a distance sufficient to ensure that each antigenic peptide properly folds. Exemplary peptide linker sequences adopt a flexible extended conformation and do not exhibit a propensity for developing an ordered secondary structure. Amino acids in flexible linker protein region may include Gly, Asn and Ser, or any permutation of amino acid sequences containing Gly, Asn and Ser. Other near neutral amino acids, such as Thr and Ala, also can be used in the linker sequence.

The term “subject” or “patient” refers to an organism, such as an animal (e.g., a human) which is the object of treatment, observation, or experiment. By way of example only, a subject includes, but is not limited to, a mammal, including, but not limited to, a human or a non-human mammal, such as a non-human primate, murine, bovine, equine, canine, ovine, or feline. A subject may also refer to a non-mammalian animal.

The term “therapeutic effect” may refer to some extent of relief of one or more of the symptoms of a disorder (e.g., a neoplasia, tumor, or infection by an infectious agent or an autoimmune disease) or its associated pathology. “Therapeutically effective amount” as used herein may refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required.

“Transposons” as used herein are segments within the chromosome that can translocate within the genome, also known as “jumping gene”. There are two different classes of transposons: class 1, or retrotransposons, that mobilize via an RNA intermediate and a “copy-and-paste” mechanism, and class II, or DNA transposons, that mobilize via excision integration, or a “cut-and-paste” mechanism (Ivics Nat Methods 2009). Bacterial, lower eukaryotic (e.g. yeast) and invertebrate transposons appear to be largely species specific, and cannot be used for efficient transposition of DNA in vertebrate cells. “Sleeping Beauty” (Ivics Cell 1997), was the first active transposon that was artificially reconstructed by sequence shuffling of inactive TEs from fish. This made it possible to successfully achieve DNA integration by transposition into vertebrate cells, including human cells. Sleeping Beauty is a class II DNA transposon belonging to the Tcl/mariner family of transposons (Ni Genomics Proteomics 2008). In the meantime, additional functional transposons have been identified or reconstructed from different species, including Drosophila, frog and even human genomes, that all have been shown to allow DNA transposition into vertebrate and also human host cell genomes. Each of these transposons have advantages and disadvantages that are related to transposition efficiency, stability of expression, genetic payload capacity etc. Exemplary class II transposases that have been created include Sleeping Beauty, PiggyBac, Frog Prince, Himarl, Passport, Minos, hAT, To11, To12, AciDs, PIF, Harbinger, Harbinger3-DR, and Hsmarl.

The terms “treat,” “treated,” “treating,” “treatment,” and the like are meant to refer to reducing, preventing, or ameliorating a disorder and/or symptoms associated therewith (e.g., a neoplasia or tumor or infectious agent or an autoimmune disease). “Treating” can refer to administration of the therapy to a subject after the onset, or suspected onset, of a disease (e.g., cancer or infection by an infectious agent or an autoimmune disease). “Treating” includes the concepts of “alleviating”, which can refer to lessening the frequency of occurrence or recurrence, or the severity, of any symptoms or other ill effects related to the disease and/or the side effects associated with therapy. The term “treating” also encompasses the concept of “managing” which refers to reducing the severity of a disease or disorder in a patient, e.g., extending the life or prolonging the survivability of a patient with the disease, or delaying its recurrence, e.g., lengthening the period of remission in a patient who had suffered from the disease. It is appreciated that, although not precluded, treating a disorder or condition does not require that the disorder, condition, or symptoms associated therewith be completely eliminated. The term “prevent”, “preventing”, “prevention” and their grammatical equivalents as used herein, can refer to avoiding or delaying the onset of symptoms associated with a disease or condition in a subject that has not developed such symptoms at the time the administering of an agent or compound commences. In certain embodiments, treating a subject or a patient as described herein comprises administering a therapeutic composition, such as a drug, a metabolite, a preventive component, a nucleic acid, a peptide, or a protein that encodes or otherwise forms a drug, a metabolite or a preventive component. In some embodiments, treating comprises administering a cell or a population of cells to a subject in need thereof. In some embodiments, treating comprises administering to the subject one or more of engineered cells described herein, e.g. one or more engineered myeloid cells, such as phagocytic cells. Treating comprises treating a disease or a condition or a syndrome, which may be a pathological disease, condition or syndrome, or a latent disease, condition or syndrome. In some cases, treating, as used herein may comprise administering a therapeutic vaccine. In some embodiments, the engineered phagocytic cell is administered to a patient or a subject. In some embodiments, a cell administered to a human subject results in reduced immunogenicity. For example, an engineered phagocytic cell may lead to no or reduced graft versus host disease (GVHD) or fratricide effect. In some embodiments, an engineered cell administered to a human subject is immunocompatible to the subject (i.e. having a matching HLA subtype that is naturally expressed in the subject). Subject specific HLA alleles or HLA genotype of a subject can be determined by any method known in the art. In exemplary embodiments, the methods include determining polymorphic gene types that can comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

The term “vector”, can refer to a nucleic acid molecule capable of autonomous replication in a host cell, and which allow for cloning of nucleic acid molecules. As known to those skilled in the art, a vector includes, but is not limited to, a plasmid, cosmid, phagemid, viral vectors, phage vectors, yeast vectors, mammalian vectors and the like. For example, a vector for exogenous gene transformation may be a plasmid. In certain embodiments, a vector comprises a nucleic acid sequence containing an origin of replication and other elements necessary for replication and/or maintenance of the nucleic acid sequence in a host cell. In some embodiments, a vector or a plasmid provided herein is an expression vector. Expression vectors are capable of directing the expression of genes and/or nucleic acid sequence to which they are operatively linked. In some embodiments, an expression vector or plasmid is in the form of circular double stranded DNA molecules. A vector or plasmid may or may not be integrated into the genome of a host cell. In some embodiments, nucleic acid sequences of a plasmid are not integrated in a genome or chromosome of the host cell after introduction. For example, the plasmid may comprise elements for transient expression or stable expression of the nucleic acid sequences, e.g. genes or open reading frames harbored by the plasmid, in a host cell. In some embodiments, a vector is a transient expression vector. In some embodiments, a vector is a stably expressed vector that replicates autonomously in a host cell. In some embodiments, nucleic acid sequences of a plasmid are integrated into a genome or chromosome of a host cell upon introduction into the host cell. Expression vectors that can be used in the methods as disclosed herein include, but are not limited to, plasmids, episomes, bacterial artificial chromosomes, yeast artificial chromosomes, bacteriophages or viral vectors. A vector can be a DNA or RNA vector. In some embodiments, a vector provide herein is a RNA vector that is capable of integrating into a host cell's genome upon introduction into the host cell (e.g., via reverse transcription), for example, a retroviral vector or a lentiviral vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

### Engineered Myeloid Cells

Myeloid cells, including macrophages, are cells derived from the myeloid lineage and belong to the innate immune system. They are derived from bone marrow stem cells which egress into the blood and can migrate into tissues. Some of their main functions include phagocytosis, the activation of T cell responses, and clearance of cellular debris and extracellular matrices. Another hallmark function of certain phagocytic myeloid cells, such as monocytes, macrophages and dendritic cells is antigen presentation. After phagocytosis, the phagocytosed component is processed intracellularly, and an the myeloid cell presents the antigens on the surface of the cell in conjunction of an MHC molecule and associated helper molecules e.g., co-stimulatory molecules that are recognized by T cells in the milieu, by which T cells are activated which sets off the cascade of antigen-specific immune response and generation of immunological memory. They also play an important role in maintaining homeostasis, and initiating and resolving inflammation.

Myeloid cells can differentiate into numerous downstream cells, including macrophages, which can display different responses ranging from pro-inflammatory to anti-inflammatory depending on the type of stimuli they receive from the surrounding microenvironment. Furthermore, tissue macrophages have been shown to play a broad regulatory and activating role on other immune cell types including effector T cells, NK cells and T regulatory cells. Macrophages have been shown to be a main immune infiltrate in inflamed tissue and may, in some cases display and immune activating influence, or, in some cases may have a broad immunosuppressive influence on the tissue.

Myeloid cells are a major cellular compartment of the immune system comprising monocytes, dendritic cells, tissue macrophages, and granulocytes. Models of cellular ontogeny, activation, differentiation, and tissue-specific functions of myeloid cells have been revisited during the last years with surprising results. However, their enormous plasticity and heterogeneity, during both homeostasis and disease, are far from understood. Although myeloid cells have many functions, including phagocytosis and their ability to activate T cells, harnessing these functions for therapeutic uses has remained elusive. Newer avenues are therefore sought for using other cell types towards development of improved therapeutics, including but not limited to T cell malignancies.

A myeloid cell can refer broadly to cells of the myeloid lineage of the hematopoietic cell system, and can exclude, for example, the lymphocytic lineage. Myeloid cells comprise, for example, cells of the granulocyte lineage and monocyte lineages. Myeloid cells are differentiated from common progenitors derived from the hematopoietic stem cells in the bone marrow. Commitment to myeloid cell lineages may be governed by activation of distinct transcription factors, and accordingly myeloid cells may be characterized as cells having a level of plasticity, which may be described as the ability to further differentiate into terminal cell types based on extracellular and intracellular stimuli. Myeloid cells can be rapidly recruited into local tissues via various chemokine receptors on their surface. Myeloid cells are responsive to various cytokines and chemokines.

A myeloid cell, for example, may be a cell that originates in the bone marrow from a hematopoietic stem cell under the influence of one or more cytokines and chemokines, such as G-CSF, GM-CSF, Flt3L, CCL2, VEGF and S100A8/9. In some embodiments, the myeloid cell is a precursor cell. In some embodiments, the myeloid cell may be a cell having characteristics of a common myeloid progenitor, or a granulocyte progenitor, a myeloblast cell, or a monocyte-dendritic cell progenitor or a combination thereof. A myeloid can include a granulocyte or a monocyte or a precursor cell thereof. A myeloid can include an immature granulocyte, an immature monocyte, an immature macrophage, an immature neutrophil, and an immature dendritic cell.

A myeloid can include a monocyte or a pre-monocytic cell or a monocyte precursor. In some cases, a myeloid cell as used herein may refer to a monocyte having an M0 phenotype, an M1 phenotype or an M2 phenotype. A myeloid can include a dendritic cell (DC), a mature DC, a monocyte derived DC, a plasmacytoid DC, a pre-dendritic cell, or a precursor of a DC. A myeloid can include a neutrophil, which may be a mature neutrophil, a neutrophil precursor, or a polymorphonucleocyte (PMN). A myeloid can include a macrophage, a monocyte-derived macrophage, a tissue macrophage, a macrophage of an M0, an M1 or an M2 phenotype. A myeloid can include a tumor infiltrating monocyte (TIM). A myeloid can include a tumor associated monocyte (TAM). A myeloid can include a myeloid derived suppressor cell (MDSC). A myeloid can include a tissue resident macrophage. A myeloid can include a tumor associated DC (TADC). Accordingly, a myeloid cell may express one or more cell surface markers, for example, CD11b, CD14, CD15, CD16, CD38, CCR5, CD66, Lox-1, CD11c, CD64, CD68, CD163, CCR2, CCR5, HLA-DR, CD1c, CD83, CD141, CD209, MHC-II, CD123, CD303, CD304, a SIGLEC family protein and a CLEC family protein. In some cases, a myeloid cell may be characterized by a high or a low expression of one or more of cell surface markers, for example, CD11b, CD14, CD15, CD16, CD66, Lox-1, CDIIc, CD64, CD68, CD163, CCR2, CCR5, HLA-DR, CD1c, CD83, CD141, CD209, MHC-II, CD123, CD303, CD304 or a combination thereof.

A myeloid cell may be involved in the process of phagocytosis. The process of phagocytosis can be closely coupled with an immune response and antigen presentation. The processing of exogenous antigens follows their uptake into professional antigen presenting cells by some type of endocytic event. Phagocytosis facilitate antigen presentation. For example, antigens from phagocytosed cells or pathogens, including cancer antigens, can be processed and presented on the cell surface of APCs.

Instant disclosure encompasses herein a population of human myeloid cells, particularly, for example, one or more various cells derived from the monocyte lineage, engineered to comprise an effective amount of a recombinant nucleic acid encoding a human autoimmune antigen. In one aspect, provided herein is a population of human monocytes comprising an effective amount a recombinant human autoimmune antigen.

Engineered myeloid cells can also be short-lived in vivo, phenotypically diverse, sensitive, plastic, and are often found to be difficult to manipulate in vitro. For example, engineered myeloid cells of the monocyte lineage in which a recombinant nucleic acid is incorporated, say for example, by transfection, or transduction, for example by a viral vector, is prone to alteration de novo, (where, by “alteration de novo” it is herein intended to convey that the alteration is independent of the identity or characteristics of the protein or polypeptide encoded by the nucleic acid, or its expression characteristics in the cell concerned), e.g., physiologically mature, differentiate, become terminally differentiated, lose plasticity, express one or more different cell surface marker, are activated differently, release one or more cytokines or chemokines distinct from its state prior to the transfection or transduction, exhibit altered phagocytic property, or even initiate cell death of the myeloid cell. In one embodiment, the instant disclosure encompasses carefully directing engineered myeloid cells of the monocytic lineage toward a physiologically controlled fate for utilization of cell in a desired immunotherapy.

In one embodiment, the recombinant nucleic acid comprises a viral vector, DNA plasmid or an RNA vector. The myeloid cell is engineered to comprise an effective amount of a recombinant nucleic acid encoding a human autoimmune antigen recombinant nucleic acid. An effective amount of the recombinant nucleic acid encoding a human autoimmune antigen recombinant nucleic acid comprises an amount that is sufficient to express the polypeptide encoded by the recombinant nucleic acid, e.g., the human autoimmune antigen. In one embodiment, the effective amount of the recombinant nucleic acid is an amount corresponding to about 1-100 copy numbers of a polynucleotide encoding the human autoimmune antigen per engineered cell. In one embodiment, the effective amount of the recombinant nucleic acid is an amount corresponding to about 1-200 copy numbers of a polynucleotide encoding the human autoimmune antigen per engineered cell. In one embodiment, the effective amount of the recombinant nucleic acid is an amount corresponding to about 1-300 copy numbers of a polynucleotide encoding the human autoimmune antigen per engineered cell. In one embodiment, the effective amount of the recombinant nucleic acid is an amount corresponding to about 1-400 copy numbers of a polynucleotide encoding the human autoimmune antigen per engineered cell. In one embodiment, the effective amount of the recombinant nucleic acid is an amount corresponding to about 1-500 copy numbers of a polynucleotide encoding the human autoimmune antigen per engineered cell. In one embodiment, the effective amount of the recombinant nucleic acid is an amount corresponding to about 1-600 copy numbers of a polynucleotide encoding the human autoimmune antigen per engineered cell. In one embodiment, the effective amount of the recombinant nucleic acid is an amount corresponding to about 1-700 copy numbers of a polynucleotide encoding the human autoimmune antigen per engineered cell. In one embodiment, the effective amount of the recombinant nucleic acid is an amount corresponding to about 1-800 copy numbers of a polynucleotide encoding the human autoimmune antigen per engineered cell. In one embodiment, the effective amount of the recombinant nucleic acid is an amount corresponding to about 1-900 copy numbers of a polynucleotide encoding the human autoimmune antigen per engineered cell. In one embodiment, the effective amount of the recombinant nucleic acid is an amount corresponding to about 1-1000 copy numbers of the human autoimmune antigen per engineered cell. In some embodiments, the effective amount of the recombinant nucleic acid is an amount corresponding to 1 copy of the polynucleotide encoding the human autoimmune antigen per engineered cell. In some embodiments, the effective amount of the recombinant nucleic acid is an amount corresponding to 2, 3, 4, 5, 6, 7, 8, 9 or 10 copies of the polynucleotide encoding the human autoimmune antigen per engineered cell. In some embodiments, the effective amount of the recombinant nucleic acid is an amount corresponding to about 10, 12, 14, 16, 18, 20, about 30, about 40, about 50 copies, about 60 copies, about 70 copies, about 70 copies, about 80 copies, about 90 copies, or about 100 copies of the polynucleotide encoding the human autoimmune antigen per engineered cell. In some embodiments, the effective amount of the recombinant nucleic acid is an amount corresponding to about 200, 300, 400, 500, 600, 700, 800, 900 or about 1000 copies of the polynucleotide encoding the human autoimmune antigen per engineered cell. In some embodiments, the effective amount of the recombinant nucleic acid is an amount corresponding to greater than 1000 copies of the polynucleotide encoding the human autoimmune antigen per engineered cell.

In some embodiments, the effective amount of the recombinant nucleic acid is an amount corresponding to an amount that results in detectable expression of the human autoimmune antigen encoded by the engineered cell.

In some embodiments, the myeloid cell is transfected, e.g., electroporated with 1 microgram of recombinant polynucleotide encoding the human autoimmune antigen per 10\hat{ }6 cells in a 1 ml suspension of appropriate media. In some embodiments, the myeloid cell is transfected, e.g., electroporated with about 1 microgram to about 10 micrograms (e.g., 1 2, 3, 4, 5, 6, 7, 8, 9 or 10 micrograms) of recombinant polynucleotide encoding the human autoimmune antigen per 10\hat{ }6 cells in a 1 ml suspension of appropriate media. In some embodiments, the myeloid cell is transfected, e.g., electroporated with approximately about 1 microgram to about 100 micrograms (e.g., 1, 5, 10, 20, 30, 40, 50, 60, 70, 80, 90 or 100 micrograms) of recombinant polynucleotide encoding the human autoimmune antigen per 10\hat{ }6 cells in a 1 ml suspension of appropriate media.

In some embodiments, the engineered myeloid cells can be manipulated in vitro, such that the engineered myeloid cell expresses the human autoimmune antigen encoded by the recombinant polynucleotide after the recombinant polynucleotide is introduced into the myeloid cell, such that the autoimmune antigen is processed intracellularly and accumulates in the phagolysosomal vesicles. The engineered myeloid cells can be further manipulated in vitro such that the cell is an apoptotic cell that is thereafter phagocytosed by a phagocytic cell in vivo, once the engineered myeloid cells are introduced into a subject in need thereof, after which the phagocytic cell in turn presents the autoimmune antigen to T cells in vivo, resulting in reducing or ameliorating the autoimmune reaction. Provided herein is a population of apoptotic human monocytes comprising an effective amount of a recombinant human autoimmune antigen in one or more vesicles.

Provided herein are engineered myeloid cells (including, but not limited to, neutrophils, monocytes, myeloid dendritic cells (mDCs), mast cells and macrophages), designed to comprise a recombinant polynucleotide encoding one or more autoimmune antigen(s), where the engineered myeloid cells can be utilized for inducing tolerance against the one or more autoimmune antigen(s). In some embodients, In some embodiments, the myeloid cell is a phagocytic and/or an antigen presenting cell. In some embodiments, the cell is a stem cell derived cell, a myeloid cell, a monocyte, a macrophage, a dendritic cell, a mast cell, a neutrophil, a microglia, or an astrocyte. In some embodiments, the cell is an M1 monocyte. In some embodiments, the cell is an M2 monocyte. In some embodiments, the cell is an M1 macrophage cell. In some embodiments, the cell is an M2 macrophage cell. In some embodiments, the cell is an M1 myeloid cell. In some embodiments, the cell is an M2 myeloid cell. In some embodiments, the myeloid cell is a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell.

In some embodiments, the myeloid cells are CD14⁺CD16⁻ human monocytes.

In some embodiments, the myeloid cells are CD14^{dim}CD16⁺ human monocytes.

In some embodiments, the myeloid cells are CD14⁺CD16⁺ human monocytes.

In some embodiments, the myeloid cells are CD14⁻CD16⁻ human monocytes

In some embodiments, the recombinant nucleic acid is DNA. In some embodiments, the recombinant nucleic acid is RNA. In some embodiments, the recombinant nucleic acid is mRNA. In some embodiments, the recombinant nucleic acid is an unmodified mRNA. In some embodiments, the recombinant nucleic acid is a modified mRNA. In some embodiments, the recombinant nucleic acid is a circRNA. In some embodiments, the recombinant nucleic acid is a tRNA. In some embodiments, the recombinant nucleic acid is a microRNA. Also provided herein is a vector comprising a recombinant nucleic acid sequence encoding one or more autoantigens described herein. In some embodiments, the vector is viral vector. In some embodiments, the viral vector is retroviral vector or a lentiviral vector. In some embodiments, the vector further comprises a promoter operably linked to at least one nucleic acid sequence encoding one or more polypeptides. In some embodiments, the vector is polycistronic. In some embodiments, each of the at least one nucleic acid sequence is operably linked to a separate promoter. In some embodiments, the vector further comprises one or more internal ribosome entry sites (IRESs). In some embodiments, the vector further comprises a 5′UTR and/or a 3′UTR flanking the at least one nucleic acid sequence encoding one or more polypeptides. In some embodiments, the vector further comprises one or more regulatory regions. In some embodiments, the RNA vector comprises a 5′UTR from a highly expressed gene. In some embodiments, the RNA vector comprises a stabilizing 3′UTR. In some embodiments, the RNA vector comprises a stabilizing 3′UTR from B-globin. In some embodiments, the RNA vector comprises a triplex forming sequence. In some embodiments, the RNA vector comprises a MascRNA-tRNA like sequence. In some embodiments, the RNA vector comprises a flavivirus sfRNA. In some embodiments, the recombinant nucleic acid comprises a signal peptide or a variant thereof. In some embodiments, the recombinant nucleic acid encodes a fusion polypeptide comprising an autoimmune polypeptide and an antigen enhancer. In some embodiments, the antigen enhancer is selected from LAMP-1/2, hsp110 and grp170, hsp70, hsp65, rab7 GTPas, PSGL-1/mIgG_{2b}, macrophage mannose receptor (MMR), and dendritic cell-specific intercellular adhesion molecule-3 grabbing non-integrin (DC-SIGN), or a MHC class I trafficking signal. In some embodiments, the RNA vector comprises a first nucleic acid sequence encoding a signal peptide, a second nucleic acid sequence encoding at least one tolerizing polypeptide, and a third nucleic acid sequence encoding an immunomodulatory polypeptide.

Also provided herein is a polypeptide encoded by the recombinant nucleic acid of a composition described herein. Also provided herein is a pharmaceutical composition comprising a composition described herein, such as a recombinant nucleic acid described herein, a vector described herein, a polypeptide described herein or a cell described herein; and a pharmaceutically acceptable excipient.

In some embodiments, the human autoimmune antigen is associated with an autoimmune disease, such as an autoimmune disease. In some embodiments, the human autoantigen is associated with any one of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki diseaseLambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease.

In some embodiments, the human autoimmune antigen is associated with a disease or condition that is selected from the group consisting of multiple sclerosis (MS), rheumatoid arthritis, systemic lupus erythematosus (lupus), inflammatory bowel disease (IBD), ulcerative colitis, Crohn's disease, type 1 diabetes mellitus, Guillain-Barre syndrome, chronic inflammatory demyelinating polyneuropathy, psoriasis, Graves' disease, Hashimoto's thyroiditis, myasthenia gravis, Addison's disease, Sjögren's syndrome, pernicious anemia, celiac disease and vasculitis.

In some embodiments, the human autoimmune antigen is associated with Celiac Disease. In some embodiments, the human autoimmune antigen is associated with Multiple Sclerosis. In some embodiments, the human autoimmune antigen is associated with Myasthenia gravis. In some embodiments, the human autoimmune antigen is associated with Type 1 Diabetes. In some embodiments, the human autoimmune antigen is associated with rheumatoid arthritis. In some embodiments, the human autoimmune antigen is associated with systemic lupus erythematosus (lupus). In some embodiments, the human autoimmune antigen is associated with inflammatory bowel disease (IBD). In some embodiments, the human autoimmune antigen is associated with ulcerative colitis. In some embodiments, the human autoimmune antigen is associated with Crohn's disease, type 1 diabetes mellitus. In some embodiments, the human autoimmune antigen is associated with chronic inflammatory demyelinating polyneuropathy. In some embodiments, the human autoimmune antigen is associated with psoriasis. In some embodiments, the human autoimmune antigen is associated with Graves' disease. In some embodiments, the human autoimmune antigen is associated with Hashimoto's thyroiditis. In some embodiments, the human autoimmune antigen is associated with Sjögren's syndrome. In some embodiments, the human autoimmune antigen is associated with pernicious anemia. In some embodiments, the human autoimmune antigen is associated with vasculitis.

In some embodiments, the human autoimmune antigen may be selected from the group consisting of MBP13-32, MBP83-99, MBP111-129, MBP146-170, MOG1-20, MOG35-55, and PLP139-15 polypeptide and fragment thereof.

In some embodiments, the RNA vector comprises a first nucleic acid sequence encoding a signal peptide, a second nucleic acid sequence encoding at least two human autoimmune antigens, and a third nucleic acid sequence encoding an immunomodulatory polypeptide. In some embodiments, the immunomodulatory polypeptide comprises a Lysosome-Associated Membrane Glycoprotein-1 (LAMP-1) polypeptide or fragment thereof. In some embodiments, the population comprises a plurality of RNA vectors comprising a plurality of human autoimmune antigens, wherein the human monocytes individually comprise one or more RNA vectors.

In some embodiments, the exogenously modified population of monocytes express the polypeptide comprising the autoantigenic peptide. In some embodiments, the recombinant polynucleotide encodes two or more autoantigenic peptides from a single protein.

In some embodiments, the exogenously modified population of monocytes express a tolerogenic peptide.

In one embodiment, provided herein are engineered myeloid cells wherein the engineered to comprise a recombinant polynucleotide encoding an allergic antigen. The engineered myeloid cells may comprise a recombinant polynucleotide encoding, for example, an antigen derived from a Stachybotrys antigen, a Stachybotrys protein or a fragment thereof, against which a subject has developed an allergic reaction. In some embodiments, the subject has uncontrolled, chronic allergic reaction, e.g., significant elevation in IgG or IgE antibodies against one or more Stachybotrys protein or a fragment thereof, for example, anti-Stachybotrys hemolysin and anti-Stachyrase-A IgG or IgE antibodies. Engineered myeloid cells are designed to express a suitable antigen encoded by a recombinant polynucleotide incorporated in the engineered cell, for example a Stachybotrys hemolysin epitope, or Stachyrase-A epitope, or a combination thereof. In some embodiments, the engineered myeloid cell is further manipulated ex vivo such that an apoptotic condition is induced in the myeloid cell. The engineered myeloid cells which is further manipulated in vitro such that the cell is an apoptotic cell is thereafter phagocytosed by a phagocytic cell in vivo, once the engineered myeloid cells are introduced into a subject in need thereof, after which the phagocytic cell in turn presents the allergic antigen to T cells in vivo, resulting in reducing or ameliorating the allergic reaction. Similarly, engineered myeloid cells may be generated comprising a recombinant polynucleotide encoding an allergic antigen from a tree pollens. For example, engineered myeloid cell may be generated comprising a recombinant polynucleotide encoding an birch pollen antigen Bet vi. For example, engineered myeloid cell may be generated comprising a recombinant polynucleotide encoding a Japanese cedar pollen (JCP) antigen.

In some embodiments, the exogenously modified population of monocytes do not present the autoantigenic peptide. Engineered myeloid cell may be generated comprising a recombinant polynucleotide encoding an food allergen. For example, engineered myeloid cell may be generated comprising a recombinant polynucleotide encoding an apple allergen, Mal d1. Specific engineered myeloid cells may be generated comprising a recombinant polynucleotide encoding one or more allergens derived from a crustacean (shell-fish). Specific engineered myeloid cells may be generated comprising a recombinant polynucleotide encoding one or more allergens derived from a nut, e.g., peanut. In some embodiments, engineered myeloid cells may be generated comprising a recombinant polynucleotide encoding gliadin or a fragment thereof. In some embodiments, engineered myeloid cells may be generated comprising a recombinant polynucleotide encoding barley or a fragment thereof. In some embodiments, engineered myeloid cells may be generated comprising a recombinant polynucleotide encoding hordein or a fragment thereof. In some embodiments, engineered myeloid cells may be generated comprising a recombinant polynucleotide encoding MBP or a fragment thereof. In some embodiments, engineered myeloid cells may be generated comprising a recombinant polynucleotide encoding MOG, or a fragment thereof. In some embodiments, engineered myeloid cells may be generated comprising a recombinant polynucleotide encoding PLP or a fragment thereof. In some embodiments, engineered myeloid cells may be generated comprising a recombinant polynucleotide encoding Insulin or a fragment thereof. In some embodiments, engineered myeloid cells may be generated comprising a recombinant polynucleotide encoding Pro-insulin, or a fragment thereof. In some embodiments, engineered myeloid cells may be generated comprising a recombinant polynucleotide encoding IGRP, or a fragment thereof. In some embodiments, engineered myeloid cells may be generated comprising a recombinant polynucleotide encoding Casein. In some embodiments, engineered myeloid cells may be generated comprising a recombinant polynucleotide encoding Factor VIII.

In some embodiments, the engineered myeloid cells express a fused polypeptide, comprising an epitope. In some embodiments, the fused polypeptide comprising the epitope further comprises a peptide or a fragment thereof that enhances presentation of the epitope by a MHC molecule. In one embodiment, the mRNA may comprise a peptide or a fragment thereof that can enhance the expression and/or function of a MHC molecule. Therefore, in some embodiments, the engineered cell may comprise an mRNA, e.g., a recombinant mRNA that comprises a sequence that encodes for an HLA/MHC enhancer, and a sequence encoding one or more autoimmune antigens, or autoantigenic epitopes. In one embodiment, the HLA/MHC enhancer may be a protein that binds to or interacts with an MHC gene transcription regulating elements, for example, a protein that interacts with the conserved SXY module in the transcription regulatory region of HLA-DR gene. In one embodiment, the HLA/MHC enhancer may be an activator of the CIITA/CBP family. In some embodiments, the HLA/MHC enhancer may be a protein or fragment thereof that can bind to the Vitamin D responsive element (VDRE) regulatory element present in promoters of certain HLA genes. In some embodiments, the HLA/MHC enhancer may comprise a histone modifying protein or fragment thereof. In one embodiment, the sequences encoding each antigen or epitope may be spaced with a sequence encoding self-cleaving peptide, such as T2A or P2A.

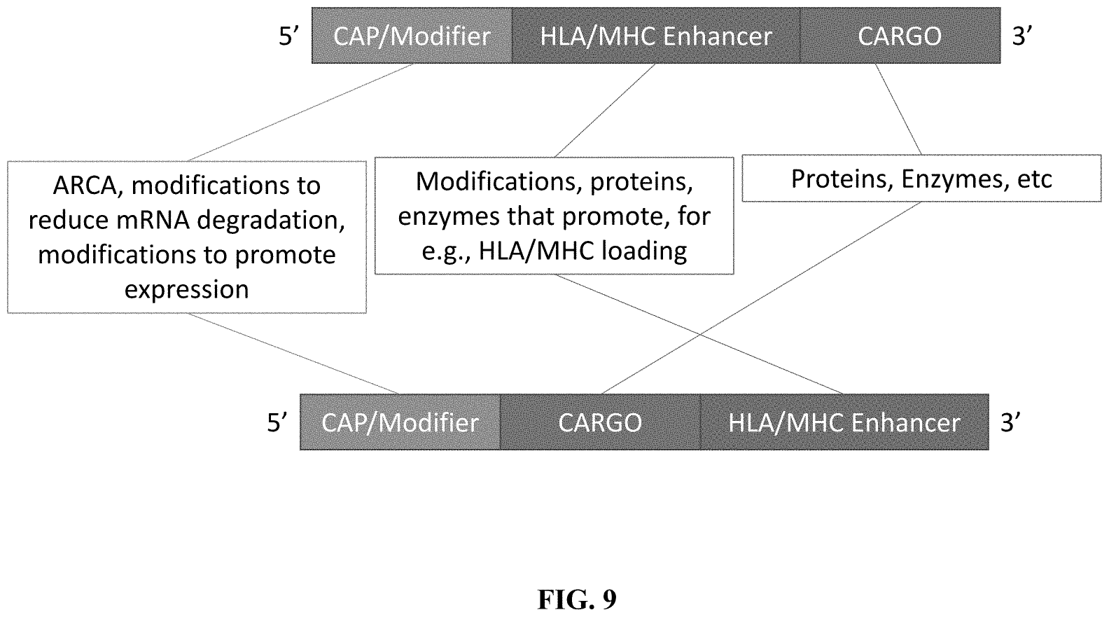

In some embodiments, the antigenic cargo sequence may be 5′ to the sequence encoding a HLA enhancer on the recombinant mRNA. In some embodiments, the antigenic cargo sequence may be 3′ to the sequence encoding a HLA enhancer on the recombinant mRNA. In some embodiments, the sequences encoding each antigen or epitope may be spaced from the adjacent sequence encoding an MHC enhancer by a sequence encoding self-cleaving peptide, such as T2A or P2A. FIG. 9 exemplifies some of the design concepts for a recombinant mRNA described herein.

In one aspect, the engineered tolerogenic myeloid cell expresses a recombinant mRNA comprising a sequence encoding an immune regulatory gene or fragment thereof. In one embodiment, an immune regulatory gene or fragment thereof may encode a cytokine, a chemokine, an immune cell signaling component, a hormone, an enzyme etc. In one embodiment, the immune regulatory gene or fragment thereof may encode a immune suppressor molecule. In one embodiment, the immune regulatory gene or fragment thereof may encode TGF beta. In one embodiment, the immune regulatory gene or fragment thereof may encode IL10. In one embodiment, the immune regulatory gene or fragment thereof may encode IL4.

In one embodiment, provided herein is an engineered myeloid cell comprising a recombinant mRNA comprising a sequence encoding one or more autoantigenic epitopes and one or more sequences encoding TGF beta, IL10 and IL4. In some embodiments, provided herein is a recombinant mRNA that comprises a sequence encoding one or more autoantigenic epitopes and one or more sequences encoding TGF beta, IL10 and IL4. In one embodiment, provided herein is a recombinant mRNA comprising a sequence encoding one or more autoantigenic epitopes and a sequence encoding human TGF beta.

In one embodiment, provided herein is an engineered myeloid cell comprising a recombinant mRNA comprising a sequence encoding one or more autoantigenic epitopes and one or more sequences encoding programmed cell death protein 1 (PD1), or programmed cell death protein ligand 1 (PDL1). In some embodiments, provided herein is a recombinant mRNA that comprises a sequence encoding one or more autoantigenic epitopes and a sequence encoding PD1, or PDL1. In some embodiments, expressing PD1 in the engineered myeloid cell influences the tolerogenic function of the engineered myeloid cell. PD1 promotes self-tolerance through modulating the activity of T-cells, activating apoptosis of antigen-specific T cells and inhibiting apoptosis of regulatory T cells. Programmed Cell Death Ligand 1 (PD-L1) is a trans-membrane protein. In some embodiments, co-expression of PDL1 acts as a co-inhibitory factor of the immune response, it can combine with PD-1 to reduce the proliferation of PD-1 positive cells, inhibit their cytokine secretion and induce apoptosis. In some embodiments, the recombinant mRNA encodes one or more autoantigenic sequences or epitopes, and a sequence encoding human PD1 or PDL1.

In one embodiment, the recombinant mRNA encodes one or more autoantigenic sequences or epitopes, a sequence encoding human TGF beta, and a sequence encoding human PD1 or PDL1.

In one embodiment, the recombinant mRNA encodes one or more autoantigenic sequences or epitopes, a sequence encoding human TGF beta or human IL10, and a sequence encoding human PD1 or PDL1.

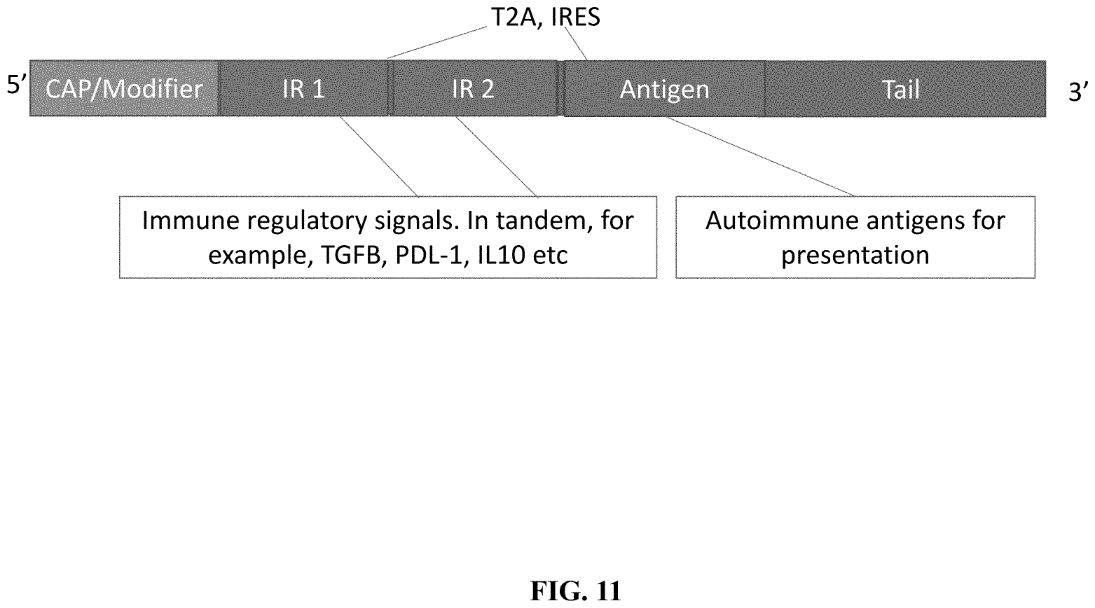

In one embodiment, the recombinant mRNA encodes one or more autoantigenic sequences or epitopes, a sequence encoding human IL10, and a sequence encoding human PD1 or PDL1. FIG. 11 exemplifies some of the mRNA designs embodied herein.

In some embodiments, the polypeptide comprising the autoantigenic peptide is a full-length protein. In some embodiments, the exogenously modified population of monocytes comprises exogenously modified monocytes that are apoptotic.

In one embodiment, provided herein are engineered myeloid cells wherein the myeloid cells are engineered to comprise a recombinant polynucleotide encoding an antigen against which an aberrant T cell activation takes place. In some embodiments, an aberrant T cell activation may be an uncontrollable T cell reaction that can ensue against a non-self-antigen derived from a foreign body, e.g., an infective pathogen, even after the pathogen is eliminated.

In one embodiment, provided herein are engineered myeloid cells wherein the myeloid cells are engineered to comprise a recombinant polynucleotide encoding a host-versus-graft-antigen in a post-transplant immunological disorder. In one embodiment, provided herein are engineered myeloid cells wherein the myeloid cells are engineered to comprise a recombinant polynucleotide encoding a graft-versus-host-antigen in a post-transplant immunological disorder.

In some embodiments, the human auto-immune antigen is a protein associated with immunogenicity to therapeutics. In one embodiment, provided herein are engineered myeloid cells wherein the myeloid cells are engineered to comprise a recombinant polynucleotide encoding an antigen against a drug or therapeutic, against which an aberrant immune response occurs in a subject. For example, in some embodiments, engineered myeloid cells may be generated comprising a recombinant polynucleotide encoding a ligand for a drug-neutralizing antibody.

In some embodiments, the myeloid cells engineered to comprise one or more antigens as described above are prepared and formulated into a therapeutic for administering into a subject, wherein the subject suffers from an aberrant immune response against the antigen. In some embodiments, the human monocytes are elutriation-purified human monocytes. In some embodiments, the human monocytes are derived from the human subject.

In some embodiments, the autoantigenic peptide is selected from the group consisting of MBP13-32, MBP83-99, MBP111-129, MBP146-170, MOG1-20, MOG35-55, PLP139-15.

In some embodiments, the recombinant polynucleotide is RNA. In some embodiments, the recombinant polynucleotide is mRNA. In some embodiments, the recombinant polynucleotide is DNA. In some embodiments, the recombinant polynucleotide is a vector. In some embodiments, the vector is a viral vector. In some embodiments, the viral vector is a lentiviral vector, an adenoviral vector or an adeno-associated viral vector.

In some embodiments, the human monocytes are elutriation-purified human monocytes.

In some embodiments, the human monocytes are derived from the human subject.

In some embodiments, the exogenously modified population of monocytes comprises exogenously modified monocytes that are apoptotic.

In some embodiments, provided herein are engineered myeloid cells wherein the engineered to comprise a recombinant polynucleotide as described in this section in the preceding paragraphs, and is further modified ex vivo by an agent to induce an apoptotic phase. In some embodiments, the cells of the exogenously modified myeloid cells are live pre-apoptotic cells and/or apoptotic cells. In some embodiments the exogenously modified myeloid cells express one or more apoptotic markers. In some embodiments, the cells exhibit surface exposure of the calreticulin CRT/ERp57 complex. In some embodiments, the cells exhibit activation of an ER stress response, such as GADD34 induction, or eIF2a and PERK activation. In some embodiments, the cells exhibit a sub-apoptotic caspase activation step. In some embodiments, the cells exhibit an apoptotic marker phosphatidyl serine (PS), which resides in the inner leaflets of the membrane in non-apoptotic healthy live cells are exposed on the outer leaflets. Such cells are detected by the dye Annexin V which binds to PS. In some embodiments, the cells are Annexin V positive, but negative for propidium iodide (PI) staining. PI staining indicates necrotic cells.

### Therapeutic Compositions

### A. Composition for Cell Therapy

### Cell Population, Modified Cells

Provided herein are therapeutic compositions comprising myeloid cells discussed above, or recombinant nucleic acid compositions comprising a sequence encoding one or more antigens.

In one aspect, provided herein is a pharmaceutical composition comprising an exogenously modified population of monocytes; and (b) a pharmaceutically acceptable excipient, diluent or carrier. In some embodiments, the exogenously modified population of monocytes comprises a recombinant polynucleotide encoding a polypeptide comprising an antigenic peptide. The antigenic peptide is an autoantigenic peptide, an autoinflammatory peptide, an allergic peptide, an anti-drug immunogenic peptide, or a GVHD antigen. In one embodiment, the polypeptide is a tolerizing polypeptide. The pharmaceutical composition comprises an immunosuppressive therapeutic. The therapeutic is for use in a subject that exhibits an autoimmune, allergic or inflammatory disease or disorder, that etiologically involves the autoantigenic peptide, an autoinflammatory peptide, an allergic peptide, an anti-drug immunogenic peptide, or a GVHD antigen.

In some embodiments, the exogenously modified population of monocytes have been engineered to comprise the recombinant polynucleotide encoding a polypeptide comprising an antigenic peptide.

In some embodiments, the exogenously modified population of monocytes have been further modified by an agent, such that when the exogenously modified population of monocytes are introduced into the subject, the antigen presenting cells (APCs) of the subject readily engulf or phagocytose monocytes of the exogenously modified population of monocytes after administration of the pharmaceutical composition.

The exogenously modified population of monocytes are monocytes obtained from a human biological sample. In some embodiments, the biological sample is peripheral blood sample, e.g., apheresis sample.

Provided herein is an exogenously modified population of monocytes comprise a population of monocytes (e.g., cells of monocytic origin, e.g. CD14+ cells), wherein the monocytes are (i) engineered to comprise a recombinant nucleic acid, and/or (ii) treated with an agent that alters the monocytic cell to be pre-apoptotic or apoptotic.

The disclosure provides a monocyte population comprising a plurality of CD14+ cells of the disclosure wherein said population comprises one or two different subclasses of CD14+ cells. The cell population of the present disclosure may comprise CD14+CD16− and/or CD14dimCD16+ and/or CD14+CD16+ and/or CD14−CD16− monocytes. At least two, at least three, at least four, at least five, or at least six or more different subclasses of CD14+ cells may be present. The subclasses may comprise monocyte subclasses, monocyte-derived suppressor cells and any other subclasses of CD14+ cells present in peripheral blood. The population may comprise all subclasses of CD14+ cells present in peripheral blood. The population may represent at least 25%, at least 30%, at least 35%, at least 40%, at least 45% or at least 50% of the CD14+ cells present in peripheral blood derived from a patient. The population may comprise classical, classical and intermediate, classical and non-classical, or classical, intermediate and non-classical modified monocytes.

In some embodiments, monocytes are obtained from whole blood, peripheral blood mononuclear cells, bone marrow, lymph node tissue, cord blood, thymus tissue, tissue from a site of infection, ascites, pleural effusion, spleen tissue.

In some embodiments, monocytes of this disclosure are obtained from peripheral blood.

In one aspect, provided herein is a population of myeloid cells, (e.g., monocytic cells) wherein the myeloid cells are engineered to comprise a recombinant polynucleotide encoding one or more autoimmune antigen(s), where the engineered myeloid cells can be utilized for inducing tolerance against the one or more autoimmune antigen(s) in a subject having an autoimmune disease or disorder. Also provided herein is a population of myeloid cells, (e.g., monocytic cells) wherein the myeloid cells are engineered to comprise a recombinant polynucleotide encoding one or more antigen(s), wherein an aberrant T cell stimulation and immune reaction against the one or more antigens is ongoing in a subject, and where the engineered myeloid cells can be utilized for inducing tolerance against the one or more antigen(s). In some embodiments, the population of myeloid cells comprise phagocytic cells, and/or an antigen presenting cells. In some embodiments, the population of cells comprise stem cell derived cell, monocytes, macrophages, and/or dendritic cells. In some embodiments, the population of myeloid cells comprise M1 monocytes. In some embodiments, the population of myeloid cells comprise M2 monocytes. In some embodiments, the population of myeloid cell comprise M1 macrophages. In some embodiments, the population of myeloid cells comprise M2 macrophage cell. In some embodiments, the population of myeloid cells comprise CD14+ cells, CD14+/CD16− cells, CD14+/CD16+ cells, CD14−/CD16+ cells, CD14−/CD16− cells, dendritic cells, M0 macrophages, M2 macrophages, M1 macrophages or mosaic myeloid cells/macrophages/dendritic cells.

In some embodiments, the population of myeloid cells are CD14⁺CD16⁻ human monocytes.

In some embodiments, the population of myeloid cells are CD14^{dim}CD16⁺ human monocytes.

In some embodiments, the population of myeloid cells are CD14⁺CD16⁺ human monocytes.

In some embodiments, the population of myeloid cells are CD14⁻CD16⁻ human monocytes

In some embodiments, the recombinant nucleic acid is DNA. In some embodiments, the recombinant nucleic acid is RNA. In some embodiments, the recombinant nucleic acid is mRNA. In some embodiments, the recombinant nucleic acid is an unmodified mRNA. In some embodiments, the recombinant nucleic acid is a modified mRNA. In some embodiments, the recombinant nucleic acid is a circRNA. In some embodiments, the recombinant nucleic acid is a tRNA. In some embodiments, the recombinant nucleic acid is a microRNA. Also provided herein is a vector comprising a recombinant nucleic acid sequence encoding one or more autoantigens described herein. In some embodiments, the vector is viral vector. In some embodiments, the viral vector is retroviral vector or a lentiviral vector. In some embodiments, the vector further comprises a promoter operably linked to at least one nucleic acid sequence encoding one or more polypeptides. In some embodiments, the vector is polycistronic. In some embodiments, each of the at least one nucleic acid sequence is operably linked to a separate promoter. In some embodiments, the vector further comprises one or more internal ribosome entry sites (IRESs). In some embodiments, the vector further comprises a 5′UTR and/or a 3′UTR flanking the at least one nucleic acid sequence encoding one or more polypeptides. In some embodiments, the vector further comprises one or more regulatory regions. In some embodiments, the RNA vector comprises a 5′UTR from a highly expressed gene. In some embodiments, the RNA vector comprises a stabilizing 3′UTR. In some embodiments, the RNA vector comprises a stabilizing 3′UTR from B-globin. In some embodiments, the RNA vector comprises a triplex forming sequence. In some embodiments, the RNA vector comprises a MascRNA-tRNA like sequence. In some embodiments, the RNA vector comprises a flavivirus sfRNA. In some embodiments, the recombinant nucleic acid comprises a signal peptide or a variant thereof. In some embodiments, the recombinant nucleic acid encodes a fusion polypeptide comprising an autoimmune polypeptide and an antigen enhancer. In some embodiments, the antigen enhancer is selected from LAMP-1/2, hsp110 and grp170, hsp70, hsp65, rab7 GTPase, PSGL-1/mIgG_{2b}, macrophage mannose receptor (MMR), and dendritic cell-specific intercellular adhesion molecule-3 grabbing non-integrin (DC-SIGN), or a MHC class I trafficking signal. In some embodiments, the RNA vector comprises a first nucleic acid sequence encoding a signal peptide, a second nucleic acid sequence encoding at least one tolerizing polypeptide, and a third nucleic acid sequence encoding an immunomodulatory polypeptide.

In some embodiments the population of monocytes are engineered to comprise the recombinant polynucleotide by transfecting, or transducing the isolated monocytes from the human biological sample with a vector comprising the recombinant polynucleotide sequence. In some embodiments the population of monocytes are engineered by electroporating the nucleic acid sequence comprising the recombinant polynucleotide into the cell population.

In one embodiment, the vector is a nucleic acid vector, for example, a plasmid, having one or more regulatory regions for expression of the recombinant polynucleotide inside the cell as discussed above. In some embodiments, the nucleic acid vector is an RNA vector. In some embodiments, the nucleic acid vector is introduced into the human monocytes by electroporation. In some embodiments, the nucleic acid vector is associated with one or more lipids for facilitating delivery into a cell. In one embodiment, the vector may be a delivery vehicle for delivery of the recombinant polynucleotide inside the cell, such as a nanoparticle. In some embodiments, the recombinant polynucleotide is an mRNA. In some embodiments, the mRNA is associated with one or more lipids for facilitating delivery into a cell. In some embodiments, the mRNA associated with one or more lipids is electroporated into the human monocytes.

In some embodiments, the polypeptide comprising the autoantigenic peptide is a full-length protein. In some embodiments, the exogenously modified population of monocytes express the polypeptide comprising the autoantigenic peptide.

In some embodiments, the population of monocytes engineered to comprise the recombinant polynucleotide are further modified by exposing the population to an agent. In some embodiments, the population of engineered monocytes are treated with the agent prior to formulating into a pharmaceutical composition. In some embodiments, the population of engineered monocytes are contacted with the agent at the time of delivery as pharmaceutical composition or at a time immediately preceding delivery. In some embodiments the population of engineered monocytes are contacted with the agent for a period of time prior to formulating as a pharmaceutical composition for delivery into a subject, or for storage at a cytostatic temperature, such as −80° C. or below. In some embodiments, the In some embodiments, the exogenously modified population of monocytes have been modified with an agent such that monocytes of the exogenously modified population of monocytes are phagocytosed, engulfed and/or recognized as apoptotic by APCs of human subject administered the pharmaceutical composition. In some embodiments, the agent comprises an apoptosis-inducing agent. In some embodiments, the agent comprises a cross-linking agent. In some embodiments, the agent comprises an agent that crosslinks lipids. In some embodiments, the agent comprises an agent that crosslinks a cell membrane. In some embodiments, the agent comprises an agent that binds with double-stranded DNA of the myeloid cells. In some embodiments, the agent comprises an agent that induces interchain cross-linking within double stranded DNA. In some embodiments, the agent comprises an agent that intercalates within double stranded DNA. In some embodiments, the agent comprises an agent that inhibits RNA synthesis. In some embodiments, the agent comprises 1-ethyl-3-(3-dimethylaminopropyl)-carbodiimide (EDC). In some embodiments, the agent comprises irradiation. In some embodiments, the agent comprises an acrylamide, a β-carboline alkaloid, anthracycline, carvacrol, p-cymene, doxorubicin, daunorubicin (DNR), idarubicin (IDA), or camptothecin (CAM), blasticidin, or cycloheximide. In some embodiments, the agent is actinomycin D. In some embodiments, the agent is specific for monocytes. In some embodiments, the agent does not induce apoptosis of the APCs that phagocytose, engulf and/or recognize the monocytes. In some embodiments, the agent is small molecule or gene. In some embodiments, the agent induces apoptosis of the monocytes of the human subject over time. In some embodiments, the agent is destroyed or rendered non-functional or degraded after the APCs phagocytose, engulf and/or recognize the monocytes.

In some embodiments, the exogenously modified population of monocytes is an exogenously modified population of CD14+ cells. In some embodiments, the exogenously modified population of monocytes is an exogenously modified population of CD14+CD16+ cells. In some embodiments, the exogenously modified population of monocytes is an exogenously modified population of CD14^{dim}CD16+ cells. In some embodiments, the exogenously modified population of monocytes is an exogenously modified population of CD14−CD16+ cells.

In some embodiments, provided herein is a human myeloid cell isolated from a biological sample, for example a leukapheresis sample, wherein the isolated myeloid cells are CD14+ cells, that are further modified by incorporating into the cell (e.g. by electroporating) an mRNA, e.g., a recombinant mRNA encoding one or more autoantigenic epitopes, and one or more sequences selected from the following: a sequence encoding an immune suppressor agent, e.g. TGF beta, a sequence encoding an HLA/MHC enhancer, or other anti-proliferative molecules such as a checkpoint component, e.g., a sequence encoding PD 1, and a sequence encoding PDL1; wherein the cell thus modified (or engineered) expresses the mRNA. When administered at the site of an autoinflammation or autoimmune reaction spot or systemically, the thus modified myeloid cells can induce suppression of inflammation.

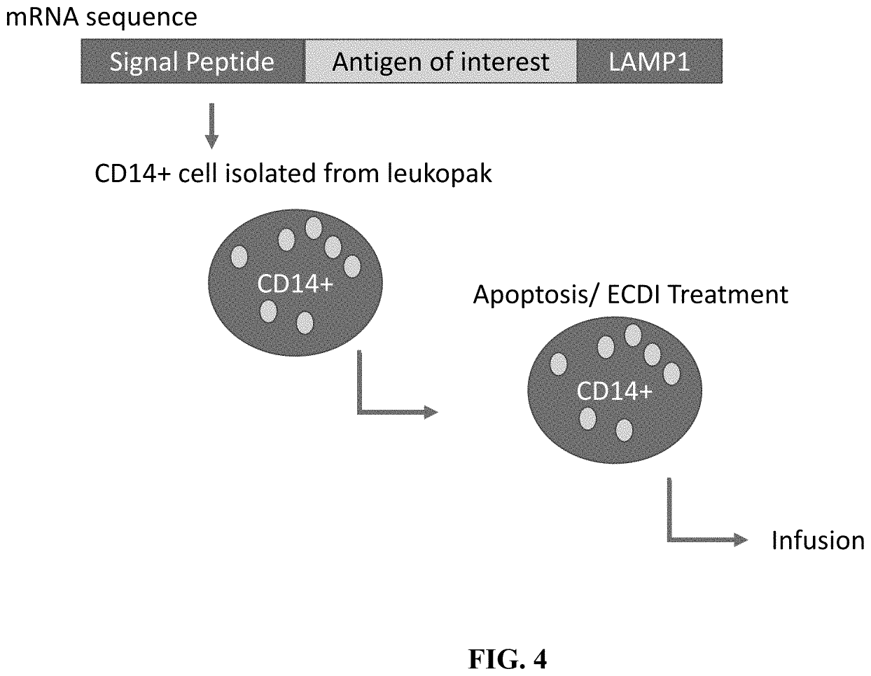

In some embodiments, provided herein is a human myeloid cell isolated from a biological sample, for example a leukapheresis sample, wherein the isolated myeloid cells are CD14+ cells, that are further modified by incorporating into the cell (e.g. by electroporating) an mRNA, e.g., a recombinant mRNA encoding one or more autoantigenic epitopes, and one or more sequences selected from the following: a sequence encoding an immune suppressor agent, e.g. TGF beta, a sequence encoding an HLA/MHC enhancer, or other anti-proliferative molecules such as a checkpoint component, e.g., a sequence encoding PD 1, and a sequence encoding PDL1; wherein the cell thus modified (or engineered) expresses the mRNA, and wherein the engineered cell is further modified by treating with an agent that induces apoptosis of the cell. The engineered and treated cell is introduced into a human in need for a cell therapy for treating a hyperimmune condition or disease, such as an autoimmune disease or an allergic disease. FIG. 4 exemplifies the concept embodied herein.

In some embodiments, the population of myeloid cells (e.g., monocytes) comprise about 10\hat{ }6 cells to about 10\hat{ }12 monocyte cells. In some embodiments, the exogenously modified population of monocytes comprises at least about 1×10\hat{ }5 exogenously modified cells. In some embodiments, the exogenously modified population of monocytes comprises at least about 1×10\hat{ }5 monocytes. In some embodiments, the exogenously modified population of monocytes comprises at least about 1×10\hat{ }5 CD14+CD16+ cells, at least about 1×10\hat{ }5 CD14^{dim}CD16+ cells, at least about 1×10\hat{ }5 CD14−CD16+ cells.

In some embodiments, the exogenously modified population of monocytes comprises at least about 1×10\hat{ }6 exogenously modified cells. In some embodiments, the exogenously modified population of monocytes comprises at least about 1×10\hat{ }6 monocytes. In some embodiments, the exogenously modified population of monocytes comprises at least about 1×10\hat{ }6 CD14+CD16+ cells, at least about 1×10\hat{ }6 CD14^{dim}CD16+ cells, at least about 1×10\hat{ }6 CD14−CD16+ cells.

In some embodiments, the exogenously modified population of monocytes comprises at least about 1×10\hat{ }7 exogenously modified cells. In some embodiments, the exogenously modified population of monocytes comprises at least about 1×10\hat{ }7 monocytes. In some embodiments, the exogenously modified population of monocytes comprises at least about 1×10\hat{ }7 CD14+CD16+ cells, at least about 1×10\hat{ }7 CD14^{dim}CD16+ cells, at least about 1×10\hat{ }7 CD14−CD16+ cells.

In some embodiments, the exogenously modified population of monocytes comprises at least about 1×10\hat{ }8 exogenously modified cells. In some embodiments, the exogenously modified population of monocytes comprises at least about 1×10\hat{ }8 monocytes. In some embodiments, the exogenously modified population of monocytes comprises at least about 1×10\hat{ }8 CD14+CD16+ cells, at least about 1×10\hat{ }8 CD14^{dim}CD16+ cells, at least about 1×10\hat{ }8 CD14−CD16+ cells.

In some embodiments, the exogenously modified population of monocytes comprises at least about 1×10\hat{ }9 exogenously modified cells. In some embodiments, the exogenously modified population of monocytes comprises at least about 1×10\hat{ }9 monocytes. In some embodiments, the exogenously modified population of monocytes comprises at least about 1×10\hat{ }9 CD14+CD16+ cells, at least about 1×10\hat{ }9 CD14^{dim}CD16+ cells, at least about 1×10\hat{ }9 CD14−CD16+ cells.

In some embodiments, the exogenously modified population of monocytes comprises at least about 1×10\hat{ }10 exogenously modified cells. In some embodiments, the exogenously modified population of monocytes comprises at least about 1×10\hat{ }10 monocytes. In some embodiments, the exogenously modified population of monocytes comprises at least about 1×10\hat{ }10 CD14+CD16+ cells, at least about 1×10\hat{ }10 CD14^{dim}CD16+ cells, at least about 1×10\hat{ }10 CD14−CD16+ cells.

In some embodiments, the exogenously modified population of monocytes comprises at least about 1×10\hat{ }11 exogenously modified cells. In some embodiments, the exogenously modified population of monocytes comprises at least about 1×10\hat{ }11 monocytes. In some embodiments, the exogenously modified population of monocytes comprises at least about 1×10\hat{ }11 CD14+CD16+ cells, at least about 1×10\hat{ }11 CD14^{dim}CD16+ cells, at least about 1×10\hat{ }11 CD14−CD16+ cells.

In some embodiments, the exogenously modified population of monocytes comprises at least about 1×10\hat{ }12 exogenously modified cells. In some embodiments, the exogenously modified population of monocytes comprises at least about 1×10\hat{ }12 monocytes. In some embodiments, the exogenously modified population of monocytes comprises at least about 1×10\hat{ }12 CD14+CD16+ cells, at least about 1×10\hat{ }12 CD14^{dim}CD16+ cells, at least about 1×10\hat{ }12 CD14−CD16+ cells.

In some embodiments, the engineered population of monocytes comprising the recombinant polynucleotide is further modified by an agent.

In some embodiments, the exogenously modified population of monocytes of the pharmaceutical composition are human monocytes that are post-proliferative.

In some embodiments, the human monocytes are pre-apoptotic or apoptotic.

For example, the isolated cell populations can have at least about 50%, 60%, 75%, 80%, 85%, 90% or 95% of cells expressing CD14.

In some embodiments, the pharmaceutically acceptable excipient, diluent or carrier comprises a particle. In some embodiments, the pharmaceutically acceptable excipient, diluent or carrier comprises a lipid nanoparticle.

In some embodiments, the pharmaceutically acceptable excipient, diluent or carrier comprises a buffer, e.g., PBS.

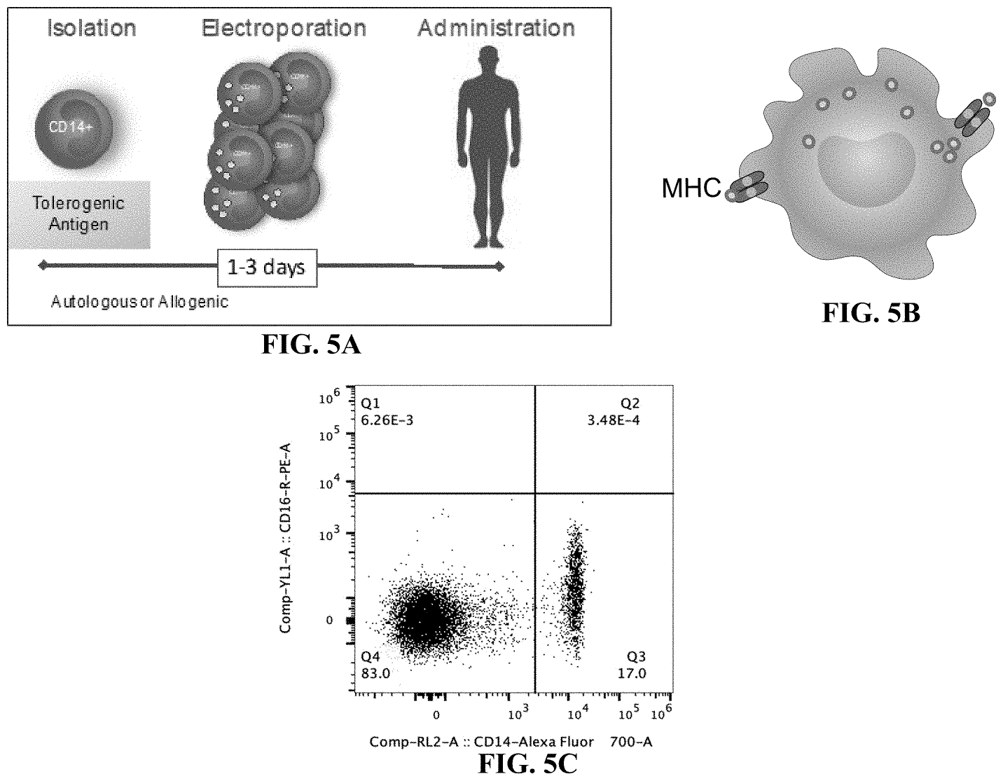

In some embodiments, provided herein is a method for preparing a cell therapy composition comprising a therapeutically effective amount of the exogenously modified population of CD14+ myeloid cells that express one or more antigenic epitopes and/or one or more immune response modulating agents, such as TGF beta, IL10, or HLA enhancer, as described in the section above, and infusing the composition into a human in need thereof. In one embodiment, it takes less than 5 days from isolating human cells to manipulating ex vivo and infusing to a human in need thereof. In some embodiment, it takes 1-4 days from isolation of cells to infusing the cell therapy product for treating a human. In some embodiment, it takes 1-3 days from isolation of cells to infusing the cell therapy product for treating a human. In some embodiments, the process from isolating a cell from a human sample to infusing the cell therapy product as described herein for treating a hyper immune condition or disease takes about 3 days. In some embodiments, the process from isolating a cell from a human sample to infusing the cell therapy product as described herein for treating a hyper immune condition or disease takes about 2 days. In some embodiments, the process from isolating a cell from a human sample to infusing the cell therapy product as described herein for treating a hyper immune condition or disease takes about 1 day. This concept is exemplified in FIGS. 5A-5C.

### Nucleic Acid Compositions

In one aspect, provided herein is a pharmaceutical composition comprising: (a) a recombinant polynucleotide encoding a polypeptide comprising an antigenic peptide; (b) an agent that modifies monocytes of a human subject administered the pharmaceutical composition such that the monocytes of the human subject are phagocytosed, engulfed and/or recognized as apoptotic by APCs of the human subject and (c) a pharmaceutically acceptable excipient, diluent or carrier. The antigenic peptide is an autoantigenic peptide, an autoinflammatory peptide, an allergic peptide, an anti-drug immunogenic peptide, or a GVHD antigen. In some embodiments, the pharmaceutical composition comprises an immunosuppressive therapeutic. The therapeutic is for use in a subject that exhibits an autoimmune, allergic or inflammatory disease or disorder, that etiologically involves the autoantigenic peptide, an autoinflammatory peptide, an allergic peptide, an anti-drug immunogenic peptide, or a GVHD antigen.

In one embodiment, the recombinant nucleic acid is DNA. In some embodiments, the recombinant nucleic acid is RNA. In some embodiments, the recombinant nucleic acid is mRNA. In some embodiments, the recombinant nucleic acid is an unmodified mRNA. In some embodiments, the recombinant nucleic acid is a modified mRNA. In some embodiments, the recombinant nucleic acid is a circRNA. In some embodiments, the recombinant nucleic acid is a self-replicating RNA. In some embodiments, the recombinant nucleic acid is a tRNA. In some embodiments, the recombinant nucleic acid is a microRNA. Also provided herein is a vector comprising a recombinant nucleic acid sequence encoding one or more autoantigens described herein. In some embodiments, the vector is viral vector. In some embodiments, the viral vector is retroviral vector or a lentiviral vector. In some embodiments, the vector further comprises a promoter operably linked to at least one nucleic acid sequence encoding one or more polypeptides. In some embodiments, the vector is polycistronic. In some embodiments, each of the at least one nucleic acid sequence is operably linked to a separate promoter. In some embodiments, the vector further comprises one or more internal ribosome entry sites (IRESs). In some embodiments, the vector further comprises a 5′UTR and/or a 3′UTR flanking the at least one nucleic acid sequence encoding one or more polypeptides. In some embodiments, the vector further comprises one or more regulatory regions. In some embodiments, the RNA vector comprises a 5′UTR from a highly expressed gene. In some embodiments, the RNA vector comprises a stabilizing 3′UTR. In some embodiments, the RNA vector comprises a stabilizing 3′UTR from B-globin. In some embodiments, the RNA vector comprises a triplex forming sequence. In some embodiments, the RNA vector comprises a MascRNA-tRNA like sequence. In some embodiments, the RNA vector comprises a flavivirus sfRNA.

In some embodiments, the nucleic acid construct, e.g., the mRNA construct, comprises one or more sequences encoding a protein or a polypeptide of interest can be designed to comprise elements that protect, prevent, inhibit or reduce degradation of the mRNA by endogenous 5′-3′ exoribonucleases, for example, Xml. Xml is a cellular enzyme in the normal RNA decay pathways that degrades 5′ monophosphorylated RNAs. However, some viral RNA structural elements are found to be particularly resistant to such RNases, for example, the Xml-resistant structure in flaviviral sfRNAs, called the ‘xrRNA’.

In some embodiments, the recombinant nucleic acid comprises a signal peptide or a variant thereof. In some embodiments, the recombinant nucleic acid encodes a fusion polypeptide comprising an autoimmune polypeptide and an antigen enhancer. In some embodiments, the antigen enhancer may comprise a sequence obtained from LAMP-1/2. In some embodiments, the antigen enhancer may comprise a sequence obtained from hsp110. In some embodiments, the antigen enhancer may comprise a sequence obtained from grp170. In some embodiments, the antigen enhancer may comprise a sequence obtained from hsp70. In some embodiments, the antigen enhancer may comprise a sequence obtained from hsp65, In some embodiments, the antigen enhancer may comprise a sequence obtained from rab7 GTPase. In some embodiments, the antigen enhancer may comprise a sequence obtained from PSGL-1/mIgG_{2b}. In some embodiments, the antigen enhancer may comprise a sequence obtained from macrophage mannose receptor (MMR). In some embodiments, the antigen enhancer may comprise a sequence obtained from the Latent 1,3-β-D-glucan, whereas Latent 1,3-β-D-glucan acts as an adjuvant for allergen-specific IgE production induced by Japanese cedar pollen exposure In some embodiments, the antigen enhancer may comprise a sequence obtained from dendritic cell-specific intercellular adhesion molecule-3 grabbing non-integrin (DC-SIGN), or a MHC class I trafficking signal. In some embodiments, the RNA vector comprises a first nucleic acid sequence encoding a signal peptide, a second nucleic acid sequence encoding at least one tolerizing polypeptide, and a third nucleic acid sequence encoding an immunomodulatory polypeptide.

In some embodiments, the mRNA is at least about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 kilobases. In some embodiments, the mRNA is a most about 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 kilobases.

In some embodiments, the mRNA comprises a sequence that inhibits or prevents degradation of the mRNA. In some embodiments, the sequence that inhibits or prevents degradation of the mRNA inhibits or prevents degradation of the mRNA by an exonuclease or an RNAse. In some embodiments, the sequence that inhibits or prevents degradation of the mRNA is a G quadruplex, pseudoknot or triplex sequence. In some embodiments, the sequence the sequence that inhibits or prevents degradation of the mRNA is an exoribonuclease-resistant RNA structure from a flaviviral RNA or an ENE element from KSV. In some embodiments, the sequence that inhibits or prevents degradation of the mRNA inhibits or prevents degradation of the mRNA by a deadenylase. In some embodiments, the sequence that inhibits or prevents degradation of the mRNA comprises non-adenosine nucleotides within or at a terminus of a poly A tail of the mRNA. In some embodiments, the sequence that inhibits or prevents degradation of the mRNA increases stability of the mRNA.

In some embodiments, one or more nucleic acid sequences are designed for integration into the genome of the myeloid cell, such as a monocyte cell. In some embodiments, the mRNA is encapsulated in a liposome or a nanoparticle designed for targeted delivery into a specific tissue or a cell type. In some embodiments, the a polynucleotide sequence capable of driving transcription of a coding sequence in a cell. Thus, promoters used in the polynucleotide constructs of the disclosure include cis-acting transcriptional control elements and regulatory sequences that are involved in regulating or modulating the timing and/or rate of transcription of a gene. For example, a promoter may be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions, developmental conditions, or drug or chemical conditions. Exemplary inducible promoter may be a doxycycline or a tetracycline inducible promoter. Tetracycline regulated promoters may be both tetracycline inducible or tetracycline repressible, called the tet-on and tet-off systems. The tet regulated systems rely on two components, i.e., a tetracycline-controlled regulator (also referred to as transactivator) (tTA or rtTA) and a tTA/rtTA-dependent promoter that controls expression of a downstream cDNA, in a tetracycline-dependent manner. tTA is a fusion protein containing the repressor of the Tn10 tetracycline-resistance operon of Escherichia coli and a carboxyl-terminal portion of protein 16 of herpes simplex virus (VP16). The tTA-dependent promoter consists of a minimal RNA polymerase II promoter fused to tet operator (tetO) sequences (an array of seven cognate operator sequences). This fusion converts the tet repressor into a strong transcriptional activator in eukaryotic cells. In the absence of tetracycline or its derivatives (such as doxycycline), tTA binds to the tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. In contrast, in the tet-ON system, a mutant form of tTA, termed rtTA, has been isolated using random mutagenesis. In contrast to tTA, rtTA is not functional in the absence of doxycycline but requires the presence of the ligand for transactivation

In some embodiments, the exogenous sequence comprises a sequence encoding an exogenous polypeptide. In some embodiments the sequence encoding an exogenous polypeptide is integrated into the genome of the myeloid cells. In some embodiments, the sequence encoding an exogenous polypeptide is not in frame with a sequence encoding an endonuclease and/or a reverse transcriptase. In some embodiments, the exogenous sequence does not comprise introns. In some embodiments, the exogenous sequence comprises a sequence encoding an exogenous polypeptide selected from the group consisting of an enzyme, a receptor, a transport protein, a structural protein, a hormone, an antibody, a contractile protein and a storage protein. In some embodiments, the exogenous sequence comprises a regulatory sequence. In some embodiments, the regulatory sequence comprises a cis-acting regulatory sequence. In some embodiments, the regulatory sequence comprises a cis-acting regulatory sequence selected from the group consisting of an enhancer, a silencer, a promoter or a response element. In some embodiments, the regulatory sequence comprises a trans-acting regulatory sequence. In some embodiments, the regulatory sequence comprises a trans-acting regulatory sequence that encodes a transcription factor. In some embodiments, the regulatory sequence comprises a suicide gene sequence.

In some embodiments, integration of the insert sequence does not adversely affect cell health. In some embodiments, the endonuclease, the reverse transcriptase or both are capable of site-specific integration of the insert sequence.

In some embodiments, the one or more nuclease domains from the heterologous protein are incorporated into the mRNA construct for site-specific integration of the mRNA comprising a cargo sequence. In some embodiments, the mRNA may comprise a CRISPR-Cas protein domain loaded with a specific guide nucleic acid, e.g., a guide RNA (gRNA) for a specific target locus. In some embodiments, the CRISPR-Cas protein is a Cas9, a Cas12a, a Cas12b, a Cas13, a CasX, or a CasY protein domain. In some embodiments, the one or more nuclease domains from the heterologous protein has target specificity

In some embodiments, the mRNA comprises a sequence encoding an additional nuclease domain or a nuclease domain that is not derived from ORF2. In some embodiments, the mRNA comprises a sequence encoding a megaTAL nuclease domain, a TALEN domain, a Cas9 domain, a zinc finger binding domain from an R2 retroelement, or a DNA binding domain that binds to repetitive sequences such as a Rep78 from AAV. In some embodiments, the endonuclease comprises a mutation that reduces activity of the endonuclease compared to the endonuclease without the mutation. In some embodiments, the endonuclease is an ORF2p endonuclease and the mutation is S228P. In some embodiments, the mRNA comprises a sequence encoding a domain that increases fidelity and/or processivity of the reverse transcriptase. In some embodiments, the reverse transcriptase is a reverse transcriptase from a retroelement other than ORF2 or reverse transcriptase that has higher fidelity and/or processivity compared to a reverse transcriptase of ORF2p. In some embodiments, the reverse transcriptase is a group II intron reverse transcriptase. In some embodiments, the group II intron reverse transcriptase is a group IIA intron reverse transcriptase, a group IIB intron reverse transcriptase, or a group IIC intron reverse transcriptase. In some embodiments, the group II intron reverse transcriptase is TGIRT-II or TGIRT-III.

In some embodiments, the mRNA comprises a sequence comprising an Alu element and/or a ribosome binding aptamer. In some embodiments, the mRNA comprises a sequence encoding a polypeptide comprising a DNA binding domain. In some embodiments, the 3′ UTR sequence is derived from a viral 3′ UTR or a beta-globin 3′ UTR.

In one aspect, provided herein is a composition comprising a recombinant mRNA or vector encoding an mRNA, wherein the mRNA comprises a human LINE-1 transposon sequence comprising a human LINE-1 transposon 5′ UTR sequence, a sequence encoding ORF1p downstream of the human LINE-1 transposon 5′ UTR sequence, an inter-ORF linker sequence downstream of the sequence encoding ORF1p,a sequence encoding ORF2p downstream of the inter-ORF linker sequence, and a 3′ UTR sequence derived from a human LINE-1 transposon downstream of the sequence encoding ORF2p; wherein the 3′ UTR sequence comprises an insert sequence, wherein the insert sequence is a reverse complement of a sequence encoding an exogenous polypeptide or a reverse complement of a sequence encoding an exogenous regulatory element.

In some embodiments, a synthetic nucleic acid is provided herein, the synthetic nucleic acid encoding a transgene, and encoding one or more components for genomic integration and/or retrotransposition.

In one aspect, provided herein is a method of integrating a nucleic acid sequence into a genome of a cell, the method comprising introducing a recombinant mRNA or a vector encoding an mRNA into the cell, wherein the mRNA comprises: an insert sequence, wherein the insert sequence comprises an exogenous sequence, or a sequence that is a reverse complement of the exogenous sequence; a 5′ UTR sequence and a 3′ UTR sequence downstream of the 5′ UTR sequence; wherein the 5′ UTR sequence or the 3′ UTR sequence comprises a binding site for a human ORF protein, and wherein the insert sequence is integrated into the genome of the cell. In some embodiments, the 5′ UTR sequence or the 3′ UTR sequence comprises a binding site for human ORF2p.

In one aspect, provided herein is a method for integrating a nucleic acid sequence into the genome of an immune cell, the method comprising introducing a recombinant mRNA or a vector encoding an mRNA, wherein the mRNA comprises an insert sequence, wherein the insert sequence comprises (i) an exogenous sequence or (ii) a sequence that is a reverse complement of the exogenous sequence; 5′ UTR sequence and a 3′ UTR sequence downstream of the 5′ UTR sequence, wherein the 5′ UTR sequence or the 3′ UTR sequence comprises an endonuclease binding site and/or a reverse transcriptase binding site, and wherein the transgene sequence is integrated into the genome of the immune cell.

A proper 5′-cap structure is important in the synthesis of functional messenger RNA. In some embodiments, the 5′-cap comprises a guanosine triphosphate arranged as GpppG at the 5′terminus of the nucleic acid. In some embodiments, the mRNA comprises a 5′ 7-methylguanosine cap, m7-GpppG. A 5′ 7-methylguanosine cap increases mRNA translational efficiency and prevents degradation of mRNA 5′-3′exonucleases. In some embodiments, the mRNA comprises “anti-reverse” cap analog (ARCA, m⁷′3′-oGpppG). Translational efficiency, however, can be markedly increased by usage of the ARCA. In some embodiments, the guanosine cap is a Cap 0 structure. In some embodiments, the guanosine cap is a Cap 1 structure. In addition to its essential role of cap-dependent initiation of protein synthesis, the mRNA cap also functions as a protective group from 5′ to 3′ exonuclease cleavage and a unique identifier for recruiting protein factors for pre-mRNA splicing, polyadenylation and nuclear export. It acts as the anchor for the recruitment of initiation factors that initiate protein synthesis and the 5′ to 3′ looping of mRNA during translation. Three enzymatic activities are required to generate the Cap 0 structure, namely, RNA triphosphatase (TPase), RNA guanylyltransferase (GTase) and guanine-N7 methyltransferase (guanine-N7 MTase). Each of these enzyme activities carries out an essential step in the conversion of the 5′ triphosphate of nascent RNA to the Cap 0 structure. RNA TPase removes the γ-phosphate from the 5′ triphosphate to generate 5′ diphosphate RNA. GTase transfers a GMP group from GTP to the 5′ diphosphate via a lysine-GMP covalent intermediate. The guanine-N7 MTase then adds a methyl group to the N7 amine of the guanine cap to form the cap 0 structure. For Cap 1 structure, m7G-specific 2′O methyltransferase (2′O MTase) methylates the +1 ribonucleotide at the 2′O position of the ribose to generate the cap 1 structure. The nuclear RNA capping enzyme interacts with the polymerase subunit of RNA polymerase II complex at phosphorylated Ser5 of the C-terminal heptad repeats. RNA guanine-N7 methyltransferase also interacts with the RNA polymerase II phosphorylated heptad repeats. In some embodiments, the cap is a G-quadruplex cap.

The poly A structure in the 3′UTR of an mRNA is an important regulator of mRNA half-life. In some embodiments, the length of the poly A tail of the mRNA construct is taken into critical consideration and designed for maximizing the expression of the protein encoded by the mRNA coding region, and the mRNA stability. In some embodiments, the nucleic acid construct comprises one or more poly A sequences. In some embodiments, the poly A sequence at the 3′UTR of the sequences encoding the one or more proteins or polypeptides comprise 20-200 adenosine nucleobases. In some embodiments, the poly A sequence comprises 30-200 adenosine nucleobases. In some embodiments, the poly A sequence comprises 50-200 adenosine nucleobases. In some embodiments, the poly A sequence comprises 80-200 adenosine nucleobases. In some embodiments, the mRNA segment comprising the sequences that encode one or more proteins or polypeptides comprises a 3′-UTR having a poly-A tail comprising about 180 adenosine nucleobases, or about 140 adenosine nucleobases, or about 120 adenosine nucleobases. In some embodiments, the poly A tail comprises about 122 adenosine nucleobases. In some embodiments, the poly A sequence comprises 50 adenosine nucleobases. In some embodiments, the poly A sequence comprises 30 adenosine nucleobases. In some embodiments, the adenosine nucleobases in the poly A tail are placed in tandem, with or without intervening non-adenosine bases. In some embodiments, one or more non-adenosine nucleobases are incorporated in the poly A tail, which confer further resistance to certain exonucleases. In some embodiments, the stretch of adenosines in poly A tail of the construct comprises one or more non-adenosine (A) nucleobase. In some embodiments, the non-A nucleobase is present at −3, −2, −1, and/or +1 position at the poly A 3′-terminal region. In some embodiments, the non-A bases comprise a guanosine (G) or a cytosine (C) or an uracil base (U). In some embodiments, the non-A base is a G. In some embodiments, the non-A base more than one, in tandem, for example, GG. In some embodiments, the modification at the 3′ end of the poly A tail with one or more non-A base is directed at disrupting the A base stacking at the poly A tail. The poly A base stacking is effective for deadenylation by various deadenylating enzymes, and therefore 3′end of poly ending in -AAAG, -AAAGA, or -AAAGGA are effective in conferring stability against deadenylation. In some organisms, a GC sequence intervening a poly A sequence is shown to effectively show down 3′-5′ exonuclease mediated decay. A modification contemplated herein comprises an intervening non-A residue, or a non-A residue duplex intervening a poly A stretch at the 3′end.

In some embodiments, a triplex structure is introduced in the 3′ UTR which effectively stalls or slows down exonuclease activity involving the 3′-end.

### B. Composition for Polynucleotide Therapy

In one aspect, provided herein is a recombinant polynucleotide that is modified for delivery in vivo for treating an autoimmune disease or condition in a human subject. In one aspect, the recombinant polynucleotide is an mRNA. In one embodiment, the mRNA comprises one or more sequences encoding one or more antigenic peptides, each comprising antigenic an epitope. The one or more antigenic peptides may be selected from autoantigens, allergens, autoinflammatory antigens depending on the need of the subject, wherein the subject suffers from an autoimmune disease, an allergic disease, an autoinflammatory disease, or a disease or a condition associated with an hyperactive immune response against the one or more antigenic epitopes. In some embodiments, the antigenic peptide is an autoantigenic peptide, an autoinflammatory peptide, an allergic peptide, an anti-drug immunogenic peptide, or a GVHD antigen, as described anywhere in the disclosure.

In some embodiments, the recombinant polynucleotide, e.g., the mRNA comprises sequence encoding an antigenic peptide, comprising a single epitope. In some embodiments, the recombinant polynucleotide, e.g., the mRNA comprises sequences encoding a plurality of antigens. In another embodiment, the mRNA comprises sequences encoding at least 2, 3, 4, 5, 6, 7, 8, 9, 10, 11, 12, 13, 14, 15, 16, 17, 18, 19, or 20 antigens. In yet another embodiment, In some embodiments, the recombinant polynucleotide, e.g., the mRNA comprises sequences encoding at least 2, 3, 4, 5, 6, 7, 8, 9, 10, 11, 12, 13, 14, 15, 16, 17, 18, 19, or 20 antigens.

In some embodiments the mRNA comprises sequences encoding antigens a cocktail of antigens to cover several possible alternative targets. For example, a cocktail of histocompatibility antigen fragments could be used to tolerize a subject in anticipation of future transplantation with an allograft of unknown phenotype. Allovariant regions of human leukocyte antigens are known in the art: e.g., Immunogenetics 29:231, 1989. In another example, a mixture of allergens may serve as inducing antigen for the treatment of atopy.

In one embodiment, the antigenic peptide or protein is an autoantigen, an alloantigen or a transplantation antigen. In yet another particular embodiment, the autoantigen is selected from the group consisting of myelin basic protein, collagen or fragments thereof, DNA, nuclear and nucleolar proteins, mitochondrial proteins and pancreatic β-cell proteins.

In some embodiments the mRNA comprises sequences encoding antigens that may include one or more of: aquaporin 4 antigens to treat neuromyelitis optica; pancreatic beta-cell antigens, insulin and GAD to treat insulin-dependent diabetes mellitus; collagen type 11, human cartilage gp 39 (HCgp39) and gp130-RAPS for use in treating rheumatoid arthritis; myelin basic protein (MBP), proteolipid protein (PLP) and myelin oligodendrocyte glycoprotein (MOG, see above) to treat multiple sclerosis; fibrillarin, and small nucleolar protein (snoRNP) to treat scleroderma; thyroid stimulating factor receptor (TSH-R) for use in treating Graves' disease; nuclear antigens, histones, glycoprotein gp70 and ribosomal proteins for use in treating systemic lupus erythematosus; pyruvate dehydrogenase dehydrolipoamide acetyltransferase (PCD-E2) for use in treating primary billiary cirrhosis; hair follicle antigens for use in treating alopecia areata; and human tropomyosin isoform 5 (hTM5) for use in treating ulcerative colitis.

In one embodiment, the epitopes are from myelin basic protein, proteolipid protein, myelin oligodendrocyte glycoprotein, an aquaporin, myelin associated glycoprotein, insulin, glutamic acid decarboxylase, gliadin, or the a3 chain of type IV collagen, or fragments, homologs, or isoforms thereof. In a further embodiment, the epitopes are from gluten, including from gliadin and/or glutenin. In one embodiment, the epitopes are from insulin homologs, such as those described in U.S. Pat. No. 8,476,228 hereby incorporated in its entirety for all purposes. In one embodiment, the gliaden epitopes are SEQ ID NOs: 13, 14, 16, 320, or 321 in U.S. Application with publication No. 20110293644, or those described in Sollid et al. (2012) Immunogenetics 65:455-460, both hereby incorporated in its entirety for all purposes. In some embodiments, an antigen or epitope is in Table 1 or 2 of U.S. Application with publication No. 20170173071.

In an embodiment, an antigen is, e.g., aggrecan, alanyl-tRNA syntetase (PL-12), alpha beta crystallin, alpha fodrin (Sptan 1), alpha-actinin, a1 antichymotrypsin, a1 antitripsin, a1 microglobulin, alsolase, aminoacyl-tRNA synthetase, an amyloid, an annexin, an apolipoprotein, aquaporin, bactericidal/permeability-increasing protein (BPI), β-globin precursor BP1, β-actin, β-lactoglobulin A, β-2-glycoprotein I, .beta.2-microglobulin, a blood group antigen, C reactive protein (CRP), calmodulin, calreticulin, cardiolipin, catalase, cathepsin B, a centromere protein, chondroitin sulfate, chromatin, collagen, a complement component, cytochrome C, cytochrome P450 2D6, cytokeratins, decorin, dermatan sulfate, DNA, DNA topoisomerase I, elastin, Epstein-Barr nuclear antigen 1 (EBNA1), elastin, entaktin, an extractable nuclear antigen, Factor I, Factor P, Factor B, Factor D, Factor H, Factor X, fibrinogen, fibronectin, formiminotransferase cyclodeaminase (LC-1), gliadin and amidated gliadin peptides (DGPs), gp210 nuclear envelope protein, GP2 (major zymogen granule membrane glycoprotein), a glutenin, glycoprotein gpllb/IIIa, glial fibrillary acidic protein (GFAP), glycated albumin, glyceraldehyde 3-phosphate dehydrogenase (GAPDH), haptoglobin A2, heat shock proteins, hemocyanin, heparin, a histone, histidyl-tRNA synthetase (Jo-1), a hordein, hyaluronidase, immunoglobulins, insulin, insulin receptor, an integrin, interstitial retinol-binding protein 3, intrinsic factor, Ku (p70/p80), lactate dehydrogenase, laminin, liver cytosol antigen type 1 (LCI), liver/kidney microsomal antigen 1 (LKM1), lysozyme, melanoma differentiation-associated protein 5 (MDAS), Mi-2 (chromodomain helicase DNA binding protein 4), a mitochondrial protein, muscarinic receptors, myelin-associated glycoprotein, myosin, myelin basic protein, myelin oligodendrocyte glycoprotein, myeloperoxidase (MPO), rheumatoid factor (IgM anti-IgG), neuron-specific enolase, nicotinic acetylcholine receptor A chain, nucleolin, a nucleoporin, nucleosome antigen, PM/ScllOO, PM/Scl 75, pancreatic β-cell antigen, pepsinogen, peroxiredoxin 1, phosphoglucose isomerase, phospholipids, phosphotidyl inositol, platelet derived growth factors, polymerase beta (POLB), potassium channel KIR4.1, proliferating cell nuclear antigen (PCNA), proteinase-3, proteolipid protein, proteoglycan, prothrombin, recoverin, rhodopsin, ribonuclease, a ribonucleoprotein, ribosomes, a ribosomal phosphoprotein, RNA, an Sm protein, SplOO nuclear protein, SRP54 (signal recognition particle 54 kDa), a secalin, selectin, smooth muscle proteins, sphingomyelin, streptococcal antigens, superoxide dismutase, synovial joint proteins, T1F1 gamma collagen, threonyl-tRNA synthetase (PL-7), tissue transglutaminase, thyroid peroxidase, thyroglobulin, thyroid stimulating hormone receptor, transferrin, triosephosphate isomerase, tubulin, tumor necrosis alpha, topoisomerase, Ul-dnRNP 68/70 kDa, Ul-snRNP A, Ul-snRNP C, U-snRNP B/B′, ubiquitin, vascular endothelial growth factor, vimentin, and vitronectin.

In some embodiments the mRNA comprises sequences encoding antigens associated with type 1 diabetes (T1D), e.g., preproinsulin, proinsulin, insulin, insulin B chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose6-phosphatase catalytic subunit related protein (IGRP), islet antigen 2 (IA2), and zinc transporter (ZnT8). See, e.g., Mallone et al. (2011) Clin. Dev. Immunol. 2011:513210; and U.S. Patent Publication No. 2017/0045529.

In some embodiments the mRNA comprises sequences encoding antigens associated with Grave's disease include, for example, thyroglobulin, thyroid peroxidase, and thyrotropin receptor (TSH-R).

In some embodiments the mRNA comprises sequences encoding antigens associated with autoimmune polyendocrine syndrome for example, 17-alpha hydroxylase, histidine decarboxylase, tryptophan hydroxylase, and tyrosine hydroxylase.

In some embodiments the mRNA comprises sequences encoding antigens associated with rheumatoid arthritis e.g., collagen, vimentin, aggregan, and fibrinogen.

In some embodiments the mRNA comprises sequences encoding antigens associated with Parkinson's disease, e.g., a-synuclein.

In some embodiments the mRNA comprises sequences encoding antigens associated with multiple sclerosis, e.g., myelin basic protein, myelin oligodendrocyte glycoprotein, and proteolipid protein.

In some embodiments the mRNA comprises sequences encoding antigens associated with celiac disease, e.g., tissue transglutaminase and gliadin. Other antigens associated with celiac disease include, e.g., secalins, hordeins, avenins, and glutenins.

In some embodiments, the mRNA composition comprising a sequence encoding a tolerogenic component, e.g., a protein, peptide, for example, and immunosuppressor enzyme, protein or immunosuppressive cytokine may be designated as a tolerogenic mRNA module. In some embodiments, one or more of such modules may be administered to a subject for tolerance induction. The therapeutic components are the mRNA modules. A reduction of inflammation, or induction of tolerance may be considered a therapeutic response or effect herein.

In some embodiments, the mRNA comprises a proteolipid protein (PLP) fused to a peptide or antigen or epitope of interest. Exemplary mRNA modules for inducing tolerance may encode a proteolipid protein (PLP) fused to a TGF-beta (PLP-TGFB), or Programed Death Ligand 1 (PLP-PDL1). In some embodiments, an exemplary mRNA modules for inducing tolerance may comprise a PLP fused to both PDL1 and TFGB, (PLP-PDL1-TGFB). In some embodiments, an exemplary mRNA modules for inducing tolerance may comprise a PLP fused to PDL1, TGFB and IL10 (PLP-PDL1-TGFB-IL10). In some embodiments, an exemplary mRNA modules for inducing tolerance may comprise PLP-Gliadin epitope fusion. A therapeutic cell composition may likewise comprise one or more of exemplary mRNA modules described herein.

In some embodiments, the mRNA comprises a sequence encoding an antigen associated with celiac disease, e.g. a gliadin antigen. In some embodiments, the mRNA comprises a sequence encoding a peptide, e.g., an epitope, an antigen or interest, and comprises a signal peptide sequence upstream and operably linked to a signal peptide.

In one embodiment, the mRNA is polycistronic.

In one embodiment, the mRNA comprises a sequence encoding one or more antigenic peptides, and a sequence encoding one or more of (a) an immune regulatory agent, (b) an MHC enhancer element, and (c) an agent that can induce apoptosis of the cell that takes up the mRNA in the body, as described in various embodiments of the disclosure.

In some embodiments, the mRNA comprises one or more regulatory sequences.

In one embodiment, the mRNA is modified.

In one embodiment, the mRNA comprises one or more modified nucleotides.

In one embodiment, the mRNA is directedly administered to the subject systemically.

In one embodiment, the mRNA is directedly administered to the subject locally, e.g., in a tissue, or a site of inflammation.

In one embodiment, the administration is intravenous, subcutaneous, or via aerosol.

In some embodiments, the lipid nanoparticle may be conjugated to a drug.

In some embodiments, the mRNA comprises a sequence that enables genomic integration of a segment of the polynucleotide sequence into a living cell, e.g., in vivo.

In some embodiments, the mRNA is at least about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 kilobases. In some embodiments, the mRNA is a most about 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 kilobases.

In some embodiments, the mRNA is modified. Modifications include (i) presence of a 5′ CAP, the 5′-cap comprises a guanosine triphosphate arranged as GpppG at the 5′terminus of the nucleic acid. In some embodiments, the mRNA comprises a 5′ 7-methylguanosine cap, m7-GpppG. In some embodiments, the mRNA comprises “anti-reverse” cap analog (ARCA, m^{7,3′-O}GpppG).

In some embodiments, the mRNA comprises a 3′UTR designed for increased stability of the mRNA. In some embodiments, the 3′UTR comprises a poly A tail. In some embodiments the poly A tail is 50-200 nucleotides long. In some embodiments, the poly A tail comprises >50 poly A residues. In some embodiments, the poly A tail comprises 60-100 adenosine residues. In some embodiments, the poly A tail comprises 80-120 adenosine residues. In some embodiments, the poly A tails comprises 100-150 adenosine residues. In some embodiments, the 3′-UTR of the mRNA comprises a 3′UTR sequence from B-globin. In some embodiments, the mRNA comprises a triplex forming sequence. In some embodiments, the mRNA comprises a MascRNA-tRNA like sequence. In some embodiments, the mRNA comprises a flavivirus sfRNA.

In some embodiments, the modification at the 3′ end of the poly A tail with one or more non-A base is directed at disrupting the A base stacking at the poly A tail. The poly A base stacking is effective for deadenylation by various deadenylating enzymes, and therefore 3′end of poly ending in -AAAG, -AAAGA, or -AAAGGA are effective in conferring stability against deadenylation. In some organisms, a GC sequence intervening a poly A sequence is shown to effectively show down 3′-5′ exonuclease mediated decay. A modification contemplated herein comprises an intervening non-A residue, or a non-A residue duplex intervening a poly A stretch at the 3′end.

In some embodiments, the mRNA comprises one or more nucleotide modifications. The modifications may include incorporation of one or more of N⁶-methyladenosine (m⁶A), N⁶,2′-O-dimethyladenosine (m⁶Am), 8-oxo-7,8-dihvdroguanosine (8-oxoG), pseudouridine (Ψ), 5-methylcytidine (m⁵C), and N⁴-acetylcytidine (ac⁴C) residues replacing the naturally occurring counterparts.

In some embodiments, the recombinant polynucleotide is an unmodified mRNA.

In one embodiment, the recombinant polynucleotide is designed for genomic integration. In some embodiments, the recombinant polynucleotide, e.g., mRNA comprises a genomic integration machinery for integrating a gene of interest or a fragment thereof, (an exogenous gene or fragment thereof) into the genome of a live cell, wherein the genomic integration machinery comprises a LINE 1 sequence, a CAS nuclease of any suitable sequence as described elsewhere in the disclosure.

In one embodiment, the mRNA additionally comprises a cell targeting moiety, or is conjugated to a cell targeting moiety. In some embodiments, the mRNA is associated in a composition that comprises a cell targeting moiety, for example, the mRNA is associated with one or more lipid compositions and a cell targeting moiety. In one example, the cell targeting moiety is conjugated to the one or more lipids associated with the mRNA. In one embodiment, the mRNA is associated with a lipid nanoparticle. In one embodiment the mRNA is encapsulated in a lipid nanoparticle. In one embodiment, the lipid nanoparticle comprises a cell targeting moiety. In one embodiment, the lipid nanoparticle comprises at least one charged lipid, e.g., a cationic lipid. In one embodiment, the lipid nanoparticle comprises a neutral lipid. In one embodiment, the lipid nanoparticle comprises a PEGylated lipid.

In some embodiments, the lipid nanoparticle is less than 1000 nm in diameter. In some embodiments, the lipid nanoparticle is about 500 nm in diameter. In some embodiments, the lipid nanoparticle is about 200 nm in diameter.

In one embodiment, the cell targeting moiety is a peptide, a ligand, an aptamer, an antibody or a fragment thereof.

In one embodiment, the cell targeting moiety is a circular peptide.

In one embodiment, the cell targeting moiety is a small molecule.

In some embodiments, the cell targeting moiety is conjugated to a component on the lipid nanoparticle.

### Methods of Preparation of Pharmaceutical Compositions for Immunotherapy

Recombinant polynucleotide constructs comprising the sequence encoding one or more antigens, such as an autoantigen are designed following known methods of molecular cloning. The recombinant polynucleotide sequence may be cloned into expression vectors, such as plasmids, or into viral vectors, such as adenoviral vectors following known techniques in molecular biology, using commercially available vectors and reagents.

In some embodiments, the mRNA is synthesized by in vitro transcription (IVT). In some embodiments, mRNA synthesis and capping may be performed in one step. Capping may occur in the same reaction mixture as IVT. In some embodiments, mRNA synthesis and capping may be performed in separate steps. mRNA thus formed by IVT is purified and then capped.

Engineered myeloid cells, such as macrophages and other phagocytic cells, can be prepared by incorporating nucleic acid sequences (e.g., mRNA, plasmids, viral constructs) encoding one or more antigens or a recombinant fusion protein into the cells using, for example, recombinant nucleic acid technology, synthetic nucleic acids, gene editing techniques (e.g., CRISPR), transduction (e.g., using viral constructs), electroporation, or nucleofection.

In some embodiments, the recombinant polynucleic acid is an mRNA. In some embodiments, the recombinant polynucleic acid is a circRNA. In some embodiments, the recombinant polynucleic acid is a viral vector. In some embodiments, the recombinant polynucleic acid is delivered via a viral vector. Also provided herein is a method of preparing a cell, the method comprising contacting a cell with a composition described herein, a vector described herein or a polypeptide described herein. In some embodiments, contacting comprises transducing. In some embodiments, contacting comprises chemical transfection, electroporation, nucleofection, or viral infection or transduction.

Also provided herein is a method of preparing a pharmaceutical composition comprising contacting a lipid to a composition described herein or a vector described herein. In some embodiments, contacting comprises forming a lipid nanoparticle.

Polynucleotides and Delivery

In some embodiments a polynucleotide, e.g., an RNA polynucleotide, is introduced or incorporated in the cell, e.g., a monocyte, by known methods of transfection, such as using lipofectamine, or calcium phosphate, or via physical means such as electroporation or nucleofection. In some embodiments the polynucleotide is introduced or incorporated in the cell by infection, a process commonly known as viral transduction. In some embodiments, a polynucleotide is introduced or incorporated into the cell via a lipid nanoparticle or a polymer nanoparticle.

Lipid nanoparticles (LNP) may comprise a polar and or a nonpolar lipid. In some embodiments, cholesterol is present in the LNPs for efficient delivery. LNPs are 100-300 nm in diameter provide efficient means of RNA delivery to various cell types. In some embodiments, LNP may be used to introduce the recombinant nucleic acids into a cell in in vitro cell culture. In some embodiments, the LNP encapsulates the nucleic acid wherein the nucleic acid is a naked DNA molecule. In some embodiments, the LNP encapsulates the nucleic acid wherein the nucleic acid is an RNA molecule. In some embodiments, the LNP encapsulates the nucleic acid wherein the nucleic acid is inserted in a vector, such as a plasmid vector. In some embodiments, the LNP encapsulates the nucleic acid wherein the nucleic acid is a circular RNA (circRNA) molecule.

It is well known that a nucleic acid, e.g., a messenger ribonucleic acid (mRNA), may be delivered inside a cell, whether in vitro, in vivo, in situ or ex vivo, to cause intracellular translation of the nucleic acid and production of an encoded polypeptide of interest. Because of their unique closed circular structure, circRNAs are more resistant to the degradation by exonuclease and have a longer half-life than their corresponding linear counterparts. As such, it is desirable to develop new and improved circRNAs which are useful in the production of polypeptides of interest. In an embodiment, circular RNA and/or methods of producing circular RNA is described in US patent applications US20160194368A1 and/or US20180169146A1.

The polynucleotide introduced or incorporated into the monocyte may be an RNA polynucleotide or a DNA polynucleotide. A DNA polynucleotide may be a plasmid. A RNA polynucleotide may be, e.g., mRNA or circular RNA. A RNA polynucleotide may be produced by transcription of a plasmid.

In some embodiments, a polynucleotide encodes an antigen or epitope or fragment thereof and a localization signal. In some embodiments, the localization signal is a signal peptide. In some embodiments, the localization signal or signal peptide directs the antigen or epitope or fragment thereof to a vesicle. In some embodiments, a localization signal or signal peptide is a GMCSF signal peptide. In some embodiments, a localization signal or signal peptide is described by SEQ ID NO: 1.

A polynucleotide may also comprise an enhancer, e.g., LAMP-1, hsp110 and grp170, hsp70, hsp65, rab7 GTPas, (PSGL-1/mIgG2b), the macrophage mannose receptor (MMR) and dendritic cell-specific intercellular adhesion molecule-3 grabbing non-integrin (DC-SIGN).

In some embodiments, a polynucleotide comprises one or more promoters, and other regulatory components, including enhancer binding sequence, initiation and terminal codons, a 5′UTR, a 3′UTR comprising a transcript stabilization element, and/or a conserved regulatory protein binding sequence.

The polynucleotides of the present invention may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed herein or as known in the art.

In an embodiment, an expression control sequence may be a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and PI 1 promoters from vaccinia virus, an elongation factor 1-alpha (EFla) promoter, early growth response 1 (EGRI), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et ah, Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer-binding site substituted (MND) promoter (Challita et al., J Virol. 69(2):748-55 (1995)).

In some embodiments, a polynucleotide contains a promoter. In some embodiments, a promoter is selected from a cytomegalovirus immediate early gene promoter (CMV), an elongation factor 1 alpha promoter (EF1-a), a phosphoglycerate kinase-1 promoter (PGK), a ubiquitin-C promoter (UBQ-C), a cytomegalovirus enhancer/chicken beta-actin promoter (CAG), polyoma enhancer/herpes simplex thymidine kinase promoter (MCI), a beta actin promoter (β-ACT), a simian virus 40 promoter (SV40), and a myeloproliferative sarcoma virus enhancer, negative control region deleted, dl587rev primer-binding site substituted (MND) promoter.

In some embodiments, a polynucleotide contains an IRES. In some embodiments, a polynucleotide encodes more than one polypeptide. In some embodiments, to achieve efficient translation of each of the plurality of polypeptides, the polynucleotide sequences can be separated by one or more IRES sequences or polynucleotide sequences encoding self-cleaving polypeptides.

In some embodiments, a polynucleotide comprises a Kozak sequence, e.g., the consensus Kozak sequence. The consensus Kozak sequence is (GCC)RCCATGG (SEQ ID NO: 6), where R is a purine (A or G) (Kozak, 1986. Cell. 44(2):283-92, and Kozak, 1987. Nucleic Acids Res. 15(20):8125-48).

In particular embodiments, expression is increased by incorporating posttranscriptional regulatory elements, efficient polyadenylation sites, and optionally, transcription termination signals into the polynucleotides. A variety of posttranscriptional regulatory elements can increase expression, e.g., woodchuck hepatitis virus posttranscriptional regulatory element (WPRE; Zufferey et al, 1999, J. Virol, 73:2886); the posttranscriptional regulatory element present in hepatitis B virus (HPRE) (Huang et al, Mol. Cell. Biol, 5:3864); and the like (Liu et al, 1995, Genes Dev., 9: 1766). In particular embodiments, vectors of the invention comprise a posttranscriptional regulatory element such as a WPRE or HPRE. In particular embodiments, vectors of the invention lack or do not comprise a posttranscriptional regulatory element (PTE) such as a WPRE or HPRE because in some instances these elements increase the risk of cellular transformation and/or do not substantially or significantly increase the amount of RNA transcript or increase RNA stability. Therefore, in some embodiments, vectors of the invention lack or do not comprise a posttranscriptional regulatory element. In other embodiments, vectors of the invention lack or do not comprise a WPRE or HPRE as an added safety measure.

In some embodiments, the polynucleotide for expression of the antigen is of a viral origin, for example a lentiviral vector, a retroviral or an adenoviral vector. In some embodiments the lentiviral vector is prepared in-house and manufactured in large scale for the purpose. In some embodiments, commercially available lentiviral vectors are utilized, as is known to one of skill in the art. In some embodiments the viral vector is an Adeno-Associated Virus (AAV) vector.

In some embodiments, a polynucleotide is a viral vector and/or is delivered to a monocyte virally. In some embodiments, retroviral vectors may be used. In some embodiments, a retroviral vector is Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) or lentivirus. In some embodiments, a lentivirus is HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In some embodiments, a monocyte is transfected or transduced with sfRNA from a flavivirus. The sfRNA may be part of the same or a separate polynucleotide as the RNA polynucleotide encoding the peptide. In some embodiments, the addition of sfRNA increases the stability of the mRNA encoding the peptide.

In some embodiments, one or more methods of incorporating the recombinant polynucleotide into the genome of the populations is contemplated.

In some embodiments, retrotransposon mediated stable integration of an exogenous nucleic acid sequence into the genome of a cell is contemplated. The method may take advantage of the random genomic integration machinery of the retrotransposon into the cell without creating an adverse effect. Methods described herein can be used for robust and versatile incorporation of an exogenous nucleic acid sequence into a cell, such that the exogenous nucleic acid is incorporated at a safe locus within the genome and is expressed without being silenced by the cell's inherent defense mechanism. The method described herein can be used to incorporate an exogenous nucleic acid that is about 1 kb, about 2 kb, about 3 kb, about 4 kb, about 5 kb, about 6 kb, about 7 kb about 8 kb, about 9 kb, about 10 kb, or more in size. In some embodiments, the exogenous nucleic acid is not incorporated within a ribosomal locus. In some embodiments, the exogenous nucleic acid is not incorporated within a ROSA26 locus, or another safe harbor locus. In some embodiments, the methods and compositions described herein can incorporate an exogenous nucleic acid sequence anywhere within the genome of the cell. Furthermore, contemplated herein is a retrotransposition system that is developed to incorporate an exogenous nucleic acid sequence into a specific predetermined site within the genome of a cell, without creating an adverse effect. The disclosed methods and compositions incorporate several mechanisms of engineering the retrotransposons for highly specific incorporation of the exogenous nucleic acid into a cell with high fidelity. Retrotransposons chosen for this purpose may be a human retrotransposon.

In some embodiments, a retrotransposable system is used to stably incorporate into the genome and express a non-endogenous nucleic acid, where the non-endogenous nucleic acid comprises retrotransposable elements within the nucleic acid sequence. In some embodiments, a cell's endogenous retrotransposable system (e.g., proteins and enzymes) is used to stably express a non-endogenous nucleic acid in the cell. In some embodiments, a cell's endogenous retrotransposable system (e.g., proteins and enzymes, such as a LINE-I retrotransposition system) is used, but may further express one or more components of the retrotransposable system to stably express a non-endogenous nucleic acid in the cell.

In some embodiments, the non-endogenous nucleic acid sequence encodes an antigenic peptide, and/or a myeloid cell modifying nucleic acid component. In some embodiments, the non-endogenous sequence encodes a gene that is activated upon reaching a microenvironment in vivo, such as a tissue, or an organ. In some embodiments, the non-endogenous sequence encodes a gene that is responsive to a microenvironment in vivo, such as a tissue, or an organ. In some embodiments, the non-endogenous sequence encodes a gene that is responsive to a drug applied to the subject. In some embodiments, the non-endogenous sequence encodes a suicide gene. In some embodiments, the non-endogenous sequence encodes a suicide gene that is activated upon receiving an external stimuli, such as a drug or a tissue microenvironment associated stimuli, e.g., hypoxia. In some embodiments, In some embodiments, the non-endogenous sequence encodes a trigger for apoptosis of the cell.

### Antigens and Compositions Containing Antigens and Engineered Monocytes

The present invention provides compositions and methods for inducing antigen-specific tolerance in a subject comprising an engineered apoptotic monocyte, and an antigen or epitope or fragment thereof. The antigen is a tolerance inducing antigen that contributes to the specificity of the tolerogenic response that is induced. The one or more antigens can act as an allergen that would otherwise induce T-cell receptor-mediated stimulation in a subject (i.e. if the subject had not been administered a composition comprising an engineered apoptotic monocyte, and an antigen or epitope or fragment thereof). In another embodiment, the antigen is not the same as the target antigen, wherein the target antigen is associated with a condition or suspected to cause a condition in a subject, wherein the target antigen can act as an allergen that would otherwise induce T-cell receptor-mediated stimulation in a subject (i.e. if the subject had not been administered a composition comprising an engineered apoptotic monocyte, and an antigen or epitope or fragment thereof).

In one embodiment, a composition can comprise a plurality of different antigens associated with the same condition. For example, the composition may comprise different antigens associated with multiple sclerosis. In another embodiment, a composition can comprise a plurality of different antigens associated with the same general condition. For example, the composition may comprise different antigens, each antigen being from a different plant, associated with a pollen allergen. In another embodiment, the composition may comprise different food antigens. In another embodiment, the composition comprises a plurality of antigens, wherein a subset of the plurality is associated with one condition and another subset is associated with a second condition.

In another embodiment, the composition comprises a plurality of different epitopes or fragments from the same antigen associated or suspected to cause a condition. In yet another embodiment, the composition comprises a plurality of different epitopes from a plurality of different antigens. The different epitopes can be immunodominant epitopes. An immunodominant epitope is a subunit of an antigen or antigen determinant that is most easily recognized by the immune system, such that the immunodominant epitope is responsible for the major immune response in a host to the antigenic determinant. The immunodominant epitope is also thought to most influence the specificity of an antibody to the epitope. Immunodominant epitopes have been identified for numerous antigens, such as described in Ota et al., Nature 346, 183-187 (1990) and Slavin et al., Autoimmunity 28, 109-120 (1998). Methods of identifying an immunodominant epitope is also known in the art, such as described in Kuwana et al., Arthritis Rheum. 46, 2742-7 (2002) and Huard et al., Int. Immunnol. 9, 1701-7 (1997). The method can comprise generating overlapping regions of an antigen and determining the specificity of an antibody to each region. For example, overlapping peptides of an antigen can be generated and epitope specificity to an antibody for the antigen is determined using ELISA. The peptides can comprise at least 3, 4, 5, 6, 7, 8, 9, 10, 11, 12, 13, 14, 15, 16, 17, 18, 19, 20, 21, 22, 23, 24, or 25 amino acids, wherein the overlap between the peptides can be at least 1, 2, 3, 4, 5, 6, 7, 8, 9, 10, 11, 12, 13, 14, 15, 16, 17, 18, 19, 20, 21, 22, 23, 24, or 25 amino acids. Sera from a subject can be used to test the specificity, such as sera from a subject with a condition.

In one embodiment, an immunodominant epitope is identified and used in a composition disclosed herein. In another embodiment, the immunodominant epitope is known in the art.

In one embodiment, a composition comprises one or more of the immunodominant epitopes, wherein each is contained in a vesicle of an engineered apoptotic monocyte. In another embodiment, a composition comprises a plurality of immunodominant epitopes, wherein the plurality is attached to a single engineered apoptotic monocyte. In another embodiment, a composition comprises at least 2, 3, 4, 5, 6, 7, 8, 9, 10, 11, 12, 13, 14, 15, 16, 17, 18, 19, or 20 immunodominant epitopes, wherein each is contained in a vesicle of an engineered apoptotic monocyte. In yet another embodiment, a composition comprises at least 2, 3, 4, 5, 6, 7, 8, 9, 10, 11, 12, 13, 14, 15, 16, 17, 18, 19, or 20 immunodominant epitopes, wherein a plurality, such as at least 2, 3, 4, 5, 6, 7, 8, 9, 10, 11, 12, 13, 14, 15, 16, 17, 18, 19, or 20 immunodominant epitopes is contained in a vesicle of a single engineered apoptotic monocyte.

The composition can comprise a plurality of immunodominant epitopes, wherein at least one of the plurality is associated with a first antigen and another one of the plurality is associated with a second antigen. The first and second antigen can be associated with the same condition, such as multiple sclerosis. In another embodiment, the first and second antigen can be associated with the same general condition, such as pollen allergy (for example, the first antigen can be of a first seed plant, and the second is of a second seed plant). In another embodiment, the first and second antigen can be different allergens. In yet another embodiment, the first and second antigen can each be associated with different conditions.

An antigen which is a bystander for the target antigen can also be used. This is an antigen which may not be immunologically related to the target antigen, but is preferentially expressed in a tissue where the target antigen is expressed. A working theory as to the effectiveness of bystander suppression is that suppression is an active cell-mediated process that down-regulates the effector arm of the immune response at the target cells. The suppressor cells are specifically stimulated by the inducer antigen at the mucosal surface, and home to a tissue site where the bystander antigen is preferentially expressed. Through an interactive or cytokine-mediated mechanism, the localized suppressor cells then down-regulate effector cells (or inducers of effector cells) in the neighborhood, regardless of what they are reactive against. If the effector cells are specific for a target different from the inducing antigen, then the result is a bystander effect. In one embodiment, one of ordinary skill need not identify or isolate a particular target antigen against which tolerance is desired in order to practice the present invention, in that a molecule preferentially expressed at the target site can be used as an inducing antigen.

In certain embodiments of this invention, the antigen is not in the same form as expressed in the individual being treated, but is a fragment or derivative thereof. Antigens include, but are not limited to, peptides based on a molecule of the appropriate specificity but adapted by fragmentation, residue substitution, labeling, conjugation, and/or fusion with peptides having other functional properties. The adaptation may be performed for any desirable purposes, including but not limited to the elimination of any undesirable property, such as toxicity; or to enhance any desirable property, such as mucosal binding, mucosal penetration, or stimulation of the tolerogenic arm of the immune response. Terms such as insulin peptide, collagen peptide, and myclin basic protein peptide, as used herein, refer not only to the intact subunit, but also to allotypic and synthetic variants, fragments, fusion peptides, conjugates, and other derivatives that contain a region that is homologous (preferably 70% identical, more preferably 80% identical and even more preferably 90% identical at the amino acid level) to at least 10 and preferably 20 consecutive amino acids of the respective molecule for which it is an analog, wherein the homologous region of the derivative shares with the respective parent molecule an ability to induce tolerance to the target antigen.

The antigen may comprise a component of an allergen. In one embodiment, administration of a composition comprising an apoptotic body or surrogate thereof and an epitope of an allergen, such as an immunodominant epitope, induces tolerance to the allergen in a subject. The allergen can be, but not limited to, an animal product, drug or therapeutic, food, insect or insect product, fungus, plant, or non-biological product. For example, the animal product can be a component of fur or dander, or dust mite. In another embodiment, the insect can be a cockroach, ant, bee, wasp, or mosquito, product therefrom. Non-biological products can include, but not be limited to, latex or a metal.

In an embodiment, a therapeutic is an allergen. The therapeutic can act as an allergen that would otherwise induce T-cell receptor-mediated stimulation in a subject that had not been administered a composition comprising an engineered apoptotic monocyte, with an epitope from the therapeutic, such as an immunodominant epitope. For example, the therapeutic can comprise an antibody or fragment thereof.

In yet another embodiment, the antigen is a component of a tissue to be transplanted. The antigen can comprise an allogeneic cell extract or endothelial cell antigen. For example, an engineered apoptotic monocyte and an epitope of a tissue to be transplanted can be administered to a subject prior, concurrent, or subsequent to receiving the tissue, such that the composition induces tolerance of the tissue in the subject thereby reducing the risk of transplant rejection in the subject or increasing transplant tolerance. The tissue may acts as an allergen that would otherwise induce T-cell receptor-mediated stimulation in the subject, such as if the subject were not administered the composition comprising an engineered apoptotic monocyte, and an epitope of a tissue to be transplanted. The tissue can be any transplanted tissue or organ, including, but not limited to, heart, heart valve, liver, lung, kidney, intestine, skin, eye, cornea, pancreas, ligament, tendon, and bone, composite tissue grafts (e.g., hand transplant, face transplant) and multiple organ transplants (e.g., heart-lung transplants, kidney-pancreas transplants).

The composition can further comprise an immunosuppressive agent, such as one known in the art. For example, it can be selected from the group consisting of, but not limited to, cyclosporins or metabolites or synthetic analogues thereof (such as Cyclosporin A), tacrolimus, rapamycin, corticosteroids, cyclophosphamide, chlorambucil, azathioprine, myclophenolate mofetil.

In one embodiment, a composition disclosed herein comprises one or more antigens or epitopes or fragments thereof, wherein each is attached to an engineered apoptotic monocyte. In another embodiment, a composition comprises a plurality of antigens, wherein the plurality is attached to a single engineered apoptotic monocyte. In another embodiment, a composition comprises at least 2, 3, 4, 5, 6, 7, 8, 9, 10, 11, 12, 13, 14, 15, 16, 17, 18, 19, or 20 antigens, wherein each is attached to an engineered apoptotic monocyte. In yet another embodiment, a composition comprises at least 2, 3, 4, 5, 6, 7, 8, 9, 10, 11, 12, 13, 14, 15, 16, 17, 18, 19, or 20 antigens, wherein a plurality, such as at least 2, 3, 4, 5, 6, 7, 8, 9, 10, 11, 12, 13, 14, 15, 16, 17, 18, 19, or 20 antigens is attached to a single engineered apoptotic monocyte.

It may also be desirable to provide a cocktail of antigens to cover several possible alternative targets. For example, a cocktail of histocompatibility antigen fragments could be used to tolerize a subject in anticipation of future transplantation with an allograft of unknown phenotype. Allovariant regions of human leukocyte antigens are known in the art: e.g., Immunogenetics 29:231, 1989. In another example, a mixture of allergens may serve as inducing antigen for the treatment of atopy.

In one embodiment, the antigenic peptide or protein is an autoantigen, an alloantigen or a transplantation antigen. In yet another particular embodiment, the autoantigen is selected from the group consisting of myelin basic protein, collagen or fragments thereof, DNA, nuclear and nucleolar proteins, mitochondrial proteins and pancreatic β-cell proteins.

Examples of antigens disease include: aquaporin 4 antigens to treat neuromyelitis optica; pancreatic beta-cell antigens, insulin and GAD to treat insulin-dependent diabetes mellitus; collagen type 11, human cartilage gp 39 (HCgp39) and gp130-RAPS for use in treating rheumatoid arthritis; myelin basic protein (MBP), proteolipid protein (PLP) and myelin oligodendrocyte glycoprotein (MOG, see above) to treat multiple sclerosis; fibrillarin, and small nucleolar protein (snoRN P) to treat scleroderma; thyroid stimulating factor receptor (TSH-R) for use in treating Graves' disease; nuclear antigens, histones, glycoprotein gp70 and ribosomal proteins for use in treating systemic lupus erythematosus; pyruvate dehydrogenase dehydrolipoamide acetyltransferase (PCD-E2) for use in treating primary billiary cirrhosis; hair follicle antigens for use in treating alopecia areata; and human tropomyosin isoform 5 (hTM5) for use in treating ulcerative colitis.

In one embodiment, the epitopes are from myelin basic protein, proteolipid protein, myelin oligodendrocyte glycoprotein, an aquaporin, myelin associated glycoprotein, insulin, glutamic acid decarboxylase, gliadin, or the a3 chain of type IV collagen, or fragments, homologs, or isoforms thereof. In a further embodiment, the epitopes are from gluten, including from gliadin and/or glutenin. In one embodiment, the epitopes are from insulin homologs, such as those described in U.S. Pat. No. 8,476,228 hereby incorporated in its entirety for all purposes. In one embodiment, the gliaden epitopes are SEQ ID NOs: 13, 14, 16, 320, or 321 in U.S. Application with publication No. 20110293644, or those described in Sollid et al. (2012) Immunogenetics 65:455-460, both hereby incorporated in its entirety for all purposes. In some embodiments, an antigen or epitope is in Table 1 or 2 of U.S. Application with publication No. 20170173071.

In an embodiment, an antigen is, e.g., aggrecan, alanyl-tRNA syntetase (PL-12), alpha beta crystallin, alpha fodrin (Sptan 1), alpha-actinin, a1 antichymotrypsin, a1 antitripsin, a1 microglobulin, alsolase, aminoacyl-tRNA synthetase, an amyloid, an annexin, an apolipoprotein, aquaporin, bactericidal/permeability-increasing protein (BPI), β-globin precursor BP1, β-actin, β-lactoglobulin A, β-2-glycoprotein I, .beta.2-microglobulin, a blood group antigen, C reactive protein (CRP), calmodulin, calreticulin, cardiolipin, catalase, cathepsin B, a centromere protein, chondroitin sulfate, chromatin, collagen, a complement component, cytochrome C, cytochrome P450 2D6, cytokeratins, decorin, dermatan sulfate, DNA, DNA topoisomerase I, elastin, Epstein-Barr nuclear antigen 1 (EBNA1), elastin, entaktin, an extractable nuclear antigen, Factor I, Factor P, Factor B, Factor D, Factor H, Factor X, fibrinogen, fibronectin, formiminotransferase cyclodeaminase (LC-1), gliadin and amidated gliadin peptides (DGPs), gp210 nuclear envelope protein, GP2 (major zymogen granule membrane glycoprotein), a glutenin, glycoprotein gpIIb/IIIa, glial fibrillary acidic protein (GFAP), glycated albumin, glyceraldehyde 3-phosphate dehydrogenase (GAPDH), haptoglobin A2, heat shock proteins, hemocyanin, heparin, a histone, histidyl-tRNA synthetase (Jo-1), a hordein, hyaluronidase, immunoglobulins, insulin, insulin receptor, an integrin, interstitial retinol-binding protein 3, intrinsic factor, Ku (p70/p80), lactate dehydrogenase, laminin, liver cytosol antigen type 1 (LCI), liver/kidney microsomal antigen 1 (LKM1), lysozyme, melanoma differentiation-associated protein 5 (MDAS), Mi-2 (chromodomain helicase DNA binding protein 4), a mitochondrial protein, muscarinic receptors, myelin-associated glycoprotein, myosin, myelin basic protein, myelin oligodendrocyte glycoprotein, myeloperoxidase (MPO), rheumatoid factor (IgM anti-IgG), neuron-specific enolase, nicotinic acetylcholine receptor A chain, nucleolin, a nucleoporin, nucleosome antigen, PM/ScllOO, PM/Scl 75, pancreatic β-cell antigen, pepsinogen, peroxiredoxin 1, phosphoglucose isomerase, phospholipids, phosphotidyl inositol, platelet derived growth factors, polymerase beta (POLB), potassium channel KIR4.1, proliferating cell nuclear antigen (PCNA), proteinase-3, proteolipid protein, proteoglycan, prothrombin, recoverin, rhodopsin, ribonuclease, a ribonucleoprotein, ribosomes, a ribosomal phosphoprotein, RNA, an Sm protein, SplOO nuclear protein, SRP54 (signal recognition particle 54 kDa), a secalin, selectin, smooth muscle proteins, sphingomyelin, streptococcal antigens, superoxide dismutase, synovial joint proteins, TIF1 gamma collagen, threonyl-tRNA synthetase (PL-7), tissue transglutaminase, thyroid peroxidase, thyroglobulin, thyroid stimulating hormone receptor, transferrin, triosephosphate isomerase, tubulin, tumor necrosis alpha, topoisomerase, Ul-dnRNP 68/70 kDa, Ul-snRNP A, Ul-snRNP C, U-snRNP B/B′, ubiquitin, vascular endothelial growth factor, vimentin, and vitronectin.

Antigens associated with type 1 diabetes (T1D) include, e.g., preproinsulin, proinsulin, insulin, insulin B chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose6-phosphatase catalytic subunit related protein (IGRP), islet antigen 2 (IA2), and zinc transporter (ZnT8). See, e.g., Mallone et al. (2011) Clin. Dev. Immunol. 2011:513210; and U.S. Patent Publication No. 2017/0045529.

Antigens associated with Grave's disease include, for example, thyroglobulin, thyroid peroxidase, and thyrotropin receptor (TSH-R).

Antigens associated with autoimmune polyendocrine syndrome include, 17-alpha hydroxylase, histidine decarboxylase, tiyptophan hydroxylase, and tyrosine hydroxylase.

Antigens associated with rheumatoid arthritis include, e.g., collagen, vimentin, aggregan, and fibrinogen.

Antigens associated with Parkinson's disease include, e.g., a-synuclein.

Antigens associated with multiple sclerosis include, e.g., myelin basic protein, myelin oligodendrocyte glycoprote, and proteolipid protein.

Antigens associated with celiac disease include, e.g., tissue transglutaminase and gliadin. Other antigens associated with celiac disease include, e.g., secalins, hordeins, avenins, and glutenins.

TABLE 1
Sequences

| Se- | | |
|---|---|---|
| quence | Descrip- | |
| number | tion | Sequence |
| 1 | GMCSF | ATGTGGCTGCAGTCTCTGC |
| | signal | TGCTGCTGGGAACAGTGGC |
| | peptide | CTGTAGCATCTCT |
| 2 | Immuno- | CTGATCCCCATCGCTGTGG |
| | modulatory | GTGGTGCCCTGGCGGGGCT |
| | peptide | GGTCCTCATCGTCCTCATC |
| | LAMP1 | GCCTACCTCgtcGGCAGGA |
| | | AGAGGAGTCACGCAGGCTA |
| | | CCAGACTATCTAG |
| 3 | T7 | TAATACGACTCACTATAGG |
| | promoter | |
| 4 | Exemplary | GGGAGACCCAAGCTGGCTA |
| | 5′ UTR | GCGTTTAAACTTAAGCTTG |
| | | CCACC |
| 5 | B-globin | CTGTGCCTTCTAGTTGCCA |
| | 3′ UTR | GCCATCTGTTGTTTGCCCC |
| | | TCCCCCGTGCCTTCCTTGA |
| | | CCCTGGAAGGTGCCACTCC |
| | | CACTGTCCTTTCCTAATAA |
| | | AATGAGGAAATTGCATCGC |
| | | ATTGTCTGAGTAGGTGTCA |
| | | TTCTATTCTGGGGGGTGGG |
| | | GTGGGGCAGGACAGCAAGG |
| | | GGGAGGATTGGGAAGACAA |
| | | TAGCAGGCATGCTGGGGAT |
| | | GCGGTGGGCTCTATGG |

In some embodiments, previously unknown peptide is encoded by a sequence as listed in Table 1.

In some embodiments, a previously unknown polynucleotide comprises a sequence as listed in Table 1. In some embodiments, a polynucleotide or a protein encoded by a polynucleotide contains a sequence with at least about 70%, 75%, 80%, 85%, 90%, 91%, 92%, 93%, 94%, 95%, 96%, 97%, 98%, 99%, or 99.5% similarity to one or more sequences disclosed herein. In some embodiments, a polynucleotide or a protein encoded by a polynucleotide contains a sequence that is identical to one or more sequences disclosed herein.

In some embodiments, monocytes are derived from a biological sample, such as a tissue or blood. In some embodiments, monocytes are derived from leukapheresis. In some embodiments, the monocytes are from the same subject who will receive the pharmaceutical composition as therapy, e.g., autologous monocytes. In some embodiments, the monocytes are heterologous, but are HLA compatible. The invention provides methods for isolating a population of monocytes that are useful to treat auto-immune disease or a symptom thereof in a human subject as described herein. As exemplified in the Examples below, the monocyte populations can be isolated from suitable biological samples obtained from a mammalian subject, e.g., peripheral blood or bone marrow. The methods of the present invention enable isolation of substantially pure (e.g., with at least 50%, 75% or 85% purity) monocyte populations from a bone marrow or a blood sample. The blood sample can be any sample that contains the bulk of white blood cells or mononuclear leukocytes from whole blood. For example, it can be whole blood or leukapheresis product from whole blood. Leukapheresis is a laboratory procedure in which white blood cells are separated from a sample of blood. Preferably, the CD14+ monocytes are present in the isolated monocyte population. CD14 is a membrane-associated glycosylphosphatidylinositol-linked protein expressed at the surface of cells, especially macrophages. Bone marrow, peripheral blood, and umbilical cord blood each include a sub-population of monocytes that express the CD14 antigen. Thus, these biological samples are preferred for isolating monocyte populations enriched for CD14+ cells in accordance with the methods disclosed herein. Preferably, the monocyte populations are isolated from human bone marrow, human peripheral blood, human umbilical cord blood or other related blood samples. Typically, the methods entail first removal the majority of red blood cells (RBCs) from the sample (“debulking”). This step is accompanied by separation of other blood cells (e.g., platelets, granulocytes and lymphocytes) and remaining red blood cells, if any, from monocytes. In some embodiments, monocyte populations of the present invention are isolated from a suitable sample such as bone marrow or peripheral blood via a method based on fluorescence-activated cell sorting (FACS). RBCs present in a biological sample (e.g., peripheral blood) from a mammalian subject are first removed in the isolation procedures. This can be accomplished by lysing RBCs with standard procedures well known in the art, e.g., ammonium chloride-based lysing method. See, e.g., Tiirikainen, Cytometry 20:341-8, 1995; and Simon et al., Immunol. Commun. 12:301-14, 1983. Alternatively, RBCs can be sedimented and mononuclear cells separated by centrifugation on ficoll. Procedures for separating red blood cells via ficoll density gradient centrifugation are described in the art, e.g., Tripodi et al., Transplantation. 11:487-8, 1971; Vissers et al., J. Immunol. Methods. 110:203-7, 1988; and Boyum et al., Scand. J. Immunol. 34:697-712, 1991. Another method suitable for debulking RBCs is by differential centrifugation using the ability of Hespan (Dupont, Dreieich, Germany) to induce red blood cell agglutination. See, e.g., Nagler et al., Exp. Hematol. 22: 1134-40, 1994; and Pick et al., Br. J. Haematol. 103:639-50, 1998. Further techniques that can be used to debulk RBCs include the use of blood cell filters. Such blood cell filters are readily available from commercial suppliers, e.g., the leukocyte depleting filter manufactured by Pall Biomedical Products Company (East Hills, N.Y.). [0047] After the removal of RBCs, remaining cells in the sample are suspended in an appropriate buffer that is suitable for the subsequent isolation step with FACS. For example, the cells can be resuspended in DPBS/0.5% BSA/2 mM EDTA. Flow cytometry is a technique for counting, examining, and sorting microscopic particles suspended in a stream of fluid. It allows simultaneous multiparametric analysis of the physical and/or chemical characteristics of single cells flowing through an optical and/or electronic detection apparatus. Typically, a beam of light (usually laser light) of a single wavelength is directed onto a hydro-dynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended cell passing through the beam scatters the light in some way, and fluorescent chemicals found in the particle or attached to the particle may be excited into emitting light at a lower frequency than the light source. This combination of scattered and fluorescent light is picked up by the detectors, and by analyzing fluctuations in brightness at each detector (one for each fluorescent emission peak) it is then possible to derive various types of information about the physical and chemical structure of each individual cell. FSC correlates with the cell volume and SSC depends on the inner complexity of the cell (i.e. shape of the nucleus, the amount and type of cytoplasmic granules or the membrane roughness). Some flow cytometers on the market have eliminated the need for fluorescence and use only light scatter for measurement. Other flow cytometers form images of each cell's fluorescence, scattered light, and transmitted light. Modern flow cytometers are able to analyze several thousand cells every second in real time, and can actively separate and isolate cells having specified properties. A flow cytometer is similar to a microscope, except that instead of producing an image of the cell, flow cytometry offers high-throughput automated quantification of set parameters. A flow cytometer has 5 main components: a flow cell-liquid stream, a light source (e.g., laser), a detector and Analogue to Digital Conversion (ADC) system which generate FSC and SSC as well as fluorescence signals, an amplification system, and a computer for analysis of the signals. The data generated by flow-cytometers can be plotted in a single dimension, to produce a histogram, or in two-dimensional dot plots or even in three dimensions. The regions on these plots can be sequentially separated, based on fluorescence intensity, by creating a series of subset extractions, termed “gates”. Specific gating protocols exist for diagnostic and clinical purposes especially in relation to hematology. The plots are often made on logarithmic scales. Because different fluorescent dyes' emission spectra overlap, signals at the detectors have to be compensated electronically as well as computationally.

Monocytes cultured from leukapheresis from Prodigy isolation are cultured at 2×10⁶ monocytes per cm² and per mL in culture bags (MACS GMP differentiation bags, Miltenyi) with GMP-grade TexMACS (Miltenyi) and 100 ng/mL M-CSF. Monocytes are cultured with 100 ng/mL GMP-compliant recombinant human M-CSF (R&D Systems). Cells are cultured in a humidified atmosphere at 37° C., with 5% CO2 for 7 days. A 50% volume media replenishment is carried out twice during culture (days 2 and 4) with 50% of the culture medium removed, then fed with fresh medium supplemented with 200 ng/mL M-CSF (to restore a final concentration of 100 ng/mL).

### Cell Harvesting:

For normal donor-derived macrophages, cells are removed from the wells at day 7 using Cell Dissociation Buffer (Gibco, Thermo Fisher) and a pastette. Cells are resuspended in PEA buffer and counted, then approximately 1×10⁶ cells per test are stained for flow cytometry. Leukapheresis-derived macrophages are removed from the culture bags at day 7 using PBS/EDTA buffer (CliniMACS buffer, Miltenyi) containing pharmaceutical grade 0.5% human albumin from serum (HAS; Alburex). Harvested cells are resuspended in excipient composed of two licensed products: 0.9% saline for infusion (Baxter) with 0.5% human albumin (Alburex).

### Flow Cytometry Characterization:

Monocyte and macrophage cell surface marker expression is analyzed using either a FACSCanto II (BD Biosciences) or MACSQuant 10 (Miltenyi) flow cytometer. Approximately 20,000 events are acquired for each sample. Cell surface expression of leukocyte markers in freshly isolated and day 7 matured cells is carried out by incubating cells with specific antibodies (final dilution 1:100). Cells are incubated for 5 min with FcR block (Miltenyi) then incubated at 4° C. for 20 min with antibody cocktails. Cells are washed in PEA, and dead cell exclusion dye DRAQ7 (BioLegend) is added at 1:100. Cells are stained for a range of surface markers as follows: CD45-VioBlue, CD14-PE or CD14-PerCP-Vio700, CD163-FITC, CD169-PE and CD16-APC (all Miltenyi), CCR2-BV421, CD206-FITC, CXCR4-PE and CD115-APC (all BioLegend), and 25F9-APC and CD115-APC (eBioscience). Both monocytes and macrophages are gated to exclude debris, doublets and dead cells using forward and side scatter and DRAQ7 dead cell discriminator (BioLegend) and analyzed using FlowJo software (Tree Star). From the initial detailed phenotyping, a panel is developed as Release Criteria (CD45-VB/CD206-FITC/CD14-PE/25F9 APC/DRAQ7) that defined the development of a functional macrophage from monocytes. Macrophages are determined as having mean fluorescence intensity (MFI) five times higher than the level on day 0 monocytes for both 25F9 and CD206. A second panel is developed which assessed other markers as part of an Extended Panel, composed of CCR2-BV421/CD163-FITC/CD169-PE/CD14-PerCP-Vio700/CD16-APC/DRAQ7), but is not used as part of the Release Criteria for the cell product.

Both monocytes and macrophages from buffy coat CD14 cells are tested for phagocytic uptake using pHRodo beads, which fluoresce only when taken into acidic endosomes. Briefly, monocytes or macrophages are cultured with 1-2 uL of pHRodo Escherichia coli bioparticles (LifeTechnologies, Thermo Fisher) for 1 h, then the medium is taken off and cells washed to remove non-phagocytosed particles. Phagocytosis is assessed using an EVOS microscope (Thermo Fisher), images captured and cellular uptake of beads quantified using ImageJ software (NIH freeware). The capacity to polarize toward defined differentiated macrophages is examined by treating day 7 macrophages with IFNγ (50 ng/mL) or IL-4 (20 ng/mL) for 48 h to induce polarization to M1 or M2 phenotype (or M[IFNγ] versus M[IL-4], respectively). After 48 h, the cells are visualized by EVOS bright-field microscopy, then harvested and phenotyped as before. Further analysis is performed on the cytokine and growth factor secretion profile of macrophages after generation and in response to inflammatory stimuli. Macrophages are generated from healthy donor buffy coats as before, and either left untreated or stimulated with TNFα (50 ng/mL, Peprotech) and polyinosinic:polycytidylic acid (poly I:C, a viral homolog which binds TLR3, 1 g/mL, Sigma) to mimic the conditions present in the inflamed liver, or lipopolysaccharide (LPS, 100 ng/mL, Sigma) plus IFNγ (50 IU/mL, Peprotech) to produce a maximal macrophage activation. Day 7 macrophages are incubated overnight and supernatants collected and spun down to remove debris, then stored at −80° C. until testing. Secretome analysis is performed using a 27-plex human cytokine kit and a 9-plex matrix metalloprotease kit run on a Magpix multiplex enzyme linked immunoassay plate reader (BioRad).

In some embodiments, the population of human monocytes are isolated from an apheresis product isolated from the first human subject. In some embodiments, the therapeutic composition is suitable for administration to a second human subject, wherein the first and second human subjects are not the same subject.

In some embodiments, the population of human monocytes are engineered by incorporating a recombinant nucleic acid into the cells. In some embodiments, the recombinant nucleic acid is an mRNA, wherein the mRNA may be incorporated in an RNA vector, or DNA, incorporated in a plasmid or a viral vector. In some embodiments, the recombinant nucleic acid is introduced into the provided population of human monocytes by electroporation. In some embodiments, the RNA vector is introduced into the provided population of human monocytes by electroporation.

In some embodiments, the engineered population is incubated for at least four hours. In some embodiments, the engineered population is incubated for up to about twenty-four hours. In some embodiments, the incubated population is contacted with the apoptosis-inducing agent for at least 5, 10, 15, 20, 30, 40, 50 minutes or 1, 2, 3, 4, 5, 6, 7, 8, 9, 10, 11, 12, 13, 14, 15, 16, 17, 18, 19, 20, 21, 22, or 23 hours or 1, 2, 3, 4, 5, 6, 7 days. In some embodiments, the incubated population is contacted with the apoptosis-inducing agent for a period time to induce apoptosis in at least 5, 10, 15, 20, 25, 30, 35, 40, 45, 50, 55, 60, 65, 70, 75, 80, 85, 90, 95, 96, 97, 98, 99, or greater than 99% of the incubated population. Monocytes can comprise apoptotic cells, non-apoptotic cells, or a combination thereof. In some cases, Monocytes comprises non-apoptotic cells. In some cases, monocytes comprise both apoptotic cells and non-apoptotic cells. In some cases, monocytes comprise apoptotic monocytes. In some embodiments, apoptotic monocytes comprise engineered apoptotic monocytes.

Apoptotic cells (e.g., ECDI-treated monocytes) can fail to stimulate activation and/or proliferation of antigen-specific normal T cell clones. Without wishing to be bound by theory, the apoptotic cells can fail to provide a co-stimulatory signal to T cells. As a result, the apoptotic cells can induce a state of long-term unresponsiveness termed anergy.

Monocytes can be made apoptotic a number of different ways. For example, the cells can be contacted with a chemical (e.g., a fixative or cross-linking agent, a cellular damaging agent, or a combination thereof), to make some or all of the cells apoptotic. In another example, the cells can be irradiated (e.g., with ultraviolet radiation, gamma radiation, etc.) to make some or all of the cells apoptotic.

Monocytes can be contacted with a chemical, such as a fixative or cross-linking agent. The contacting can make some or all of the cells apoptotic. Suitable fixatives or cross-linking agents include, but are not limited to: an amine-to-amine crosslinker, a sulfhydryl-to-sulfhydryl crosslinker, an amine-to-sulfhydryl crosslinker, an in vivo crosslinker, a sulfhydryl-to-carbohydrate crosslinker, a photoreactive crosslinker, a chemoselective ligation crosslinking agent, a carboxyl-to-amine crosslinker, a carbodiimide, genipin, acrylic aldehyde, diformyl, osmium tetroxide, a diimidoester, mercuric chloride, zinc sulphate, zinc chloride, trinitrophenol (picric acid), potassium dichromate, ethanol, methanol, acetone, acetic acid, or a combination thereof.

The mechanism of action of monocytes treated or contacted with a crosslinking agent e.g. EDCI, is not fully understood, but involves covalently linking amino- and carboxy groups of cell surface molecules, leading to subsequent programmed cell death.

The dose of crosslinking agent used to trigger apoptosis may be titrated to obtain a maximum benefit to the subject, including safety and efficacy. A final concentration of a cross-linking agent of less than 15 mg/ml, preferably less than 10 mg/ml, even more preferably less than 5 mg/ml, even more preferably of about 3 mg/ml may be used. The optimal dose may also vary by subject. The person skilled in the art knows how to determine the optimal dose of a crosslinking agent.

Monocytes can be contacted with a carbodiimide, or a carbodiimide derivative. Treatment with a carbodiimide can chemically crosslink free amine and carboxyl groups, and can effectively induce apoptosis in cells, organs, and/or tissues. The contacting can be for a predetermined amount of time. The contacting can make some or all of the cells apoptotic. In one embodiment, said carbodiimide comprises 1-ethyl-3-(3-dimethylaminopropyl)-carbodiimide (ECDI); N,N′-diisopropylcarbodiimide (DIC); N,N′-dicyclohexylcarbodiimide (DCC), or combination thereof. In a preferred embodiment, said carbodiimide comprises 1-ethyl-3-(3-dimethylaminopropyl)-carbodiimide (ECDI). In some embodiments, the carbodiimide can comprise ethylcarbodiimide; ethylene carbodiimide; diisopropylcarbodiimide (DIC); dicyclohexylcurbodiimide (DCC); 1-ethyl-3-(3-dimethylaminopropyl)-carbodiimide (EDCI, EDC, ECDI, or EDAC); or a combination thereof. In some cases, the carbodiimide comprises ethylcarbodiimide. In some cases, the carbodiimide comprises ethylene carbodiimide. In some cases, the carbodiimide comprises diisopropylcarbodiimide (DIC). In some cases, the carbodiimide comprises dicyclohexylcarbodiimide (DCC). In some cases, the carbodiimide comprises 1-ethyl-3-(3-dimethylaminopropyl)-carbodiimide (EDCI, EDC, ECDI, or EDAC). In some cases, the tolerizing vaccine comprises cells treated with EDCI derivatives and/or functionalized EDCI.

Monocytes can be contacted with a diimidoester. The contacting can be for a pre-determined time. The contacting can make some or all of the cells apoptotic. The diimidoester can comprise cyanuric chloride; diisocyanate; diethylpyrocarbonate (DEPC) or diethyl dicarbonate; a maleimide; benzoquinone; or a combination thereof. Monocytes can be contacted with an amine-to-amine crosslinker. The contacting can be for a pre-determined time. The contacting can make some or all of the cells apoptotic. In some cases, the amine-to-amine-crosslinker comprises disuccinimidyl glutarate (DSG); disuccinimidyl suberate (DSS); bis(sulfosuccinimidyl)suberate (BS3); tris-(succinimidyl) aminotriacetate (TSAT); BS(PEG)5; BS(PEG)9; dithiobis(succinimidyl propionate) (DSP); 3,3′-dithiobis(sulfosuccinimidyl propionate) (DTSSP); disuccinimidyl tartrate (DST); bis(2-(succinimidooxycarbonyloxy)ethyl)sulfone (BSOCOES); ethylene glycol bis(succinimidyl succinate) (EGS); sulfo-EGS; or any combination thereof. In some cases, the amine-to-amine crosslinker comprises an imidoester, such as dimethyl adipimidate (DMA); dimethyl pimelimidate (DMP); dimethyl suberimidate (DMS); dimethyl 3,3′-dithiobispropionimidate (DTBP); or any combination thereof. In some cases, the amine-to-amine crosslinker comprises a difluoro, such as 1,5-difluoro-2,4-dinitrobenzene (DFDNB).

Monocytes can be contacted with a sulfhydryl-to-sulfhydryl crosslinker. The contacting can be for a pre-determined time. The contacting can make some or all of the cells apoptotic. In some cases, the sulfhydryl-to-sulfhydryl crosslinker comprises a maleimide, such as bismaleimidoethane (BMOE); 1,4-bismaleimidobutane (BMB); bismaleimidohexane (BMH); tris(2-maleimidoethyl)amine (TMEA); BM(PEG)2 (such as 1,8-bismaleimido-diethyleneglycol); BM(PEG)3 (such as 1,1 1-bismaleimido-tri ethyleneglycol), dithiobismaleimidoethane (DTME); or any combination thereof.

Monocytes can be contacted with an amine-to-sulfhydryl crosslinker. The contacting can be for a pre-determined time. The contacting can make some or all of the cells apoptotic. In some cases, the amine-to-sulfhydryl crosslinker comprises a NHS-haloacetyl crosslinker, a NHS-maleimide, a NHS-pyridyldithiol crosslinker, a sulfosuccinimidyl 4-(N-maleimidomethyl)cyclohexane-1-carboxylate (SMCC) crosslinker, or any combination thereof. The NHS-haloacetyl crosslinkers can comprise succinimidyl iodoacetate (SIA); succinimidyl 3-(bromoacetamido)propionate (SBAP); succinimidyl (4-iodoacetyl)aminobenzoate (SIAB); sulfo-SIAB; or a combination thereof. The NHS-maleimide can comprise N-a-maleimidoacet-oxysuccinimide ester (AMAS); N-b-maleimidopropyl-oxysuccinimide ester (BMPS); N-g-maleimidobutyryl-oxysuccinimide ester (GMBS); sulfo-GMBS; m-maleimidobenzoyl-N-hydrosuccinimide ester (MBS); sulfo-MBS; SMCC; sulfo-SMCC; N-e-malemidocaproyl-oxysuccinimide ester (EMCS); sulfo-EMCS; succinimidyl 4-(p-maleimidophenyl)butyrate (SMPB); sulfo-SMPB; succinimidyl 6-((beta-maleimidopropionamido)hexanoate) (SMPH); sulfosuccinimidyl 4-(N-maleimidomethyl)cyclohexane-1-carboxy-(6-amidocaproate) (LC-SMCC); N-K-maleimidoundecanoyl-oxysulfosuccinimide ester (sulfo-KMETS); or a combination thereof. The NHS-pyridyldithiol crosslinker can comprise succinimidyl 3-(2-pyridyldithio)propionate (SPDP), succinimidyl 6-(3(2-pyridyldithio)propionamido)hexanoate (LC-SPDP), sulfo-LC-SPDP, or 4-succinimidyloxycarbonyl-alpha-methyl-a(2-pyridyldithio)tolune (SMPT).

Monocytes can be contacted with a sulfhydryl-to-carbohydrate crosslinker. The contacting can be for a pre-determined time. The contacting can make some or all of the cells apoptotic. In some cases, the sulfhydryl-to-carbohydrate crosslinker comprises (N-b-maleimidopropionic acid hydrazide (BMPH), N-e-maleimidocaproic acid hydrazide (EMCH), 4-(4-N-maleimidophenyl)butyric acid hydrazide (MPBH), N-K-maleimidoundecanoic acid hydrazide (KMUH), 3-(2-pyridyldithio)propionyl hydrazide (PDPH), or any combination thereof.

In some cases, the carboxyl-to-amine crosslinker is dicyclohexylcarbodiimide (DCC), 1-ethyl-3-(3-dimethylaminopropyl)-carbodiimide (EDCI, EDC, or EDAC), N-hydroxysuccinimide (NHS), sulfo-NHS, or any combination thereof.

Monocytes can be contacted with a photoreactive crosslinker. The contacting can be for a pre-determined time. The contacting can make some or all of the cells apoptotic. In some cases, the photoreactive crosslinker comprises a NHS ester/aryl azide, a NHS ester/diazirine, or a combination thereof. The NHS ester/aryl azide can comprise N-5-azido-2-nitrobenzoyloxysuccinimide (ANB-NOS), sulfo-SANPAH, or a combination thereof. The NHS ester/diazirine can comprise SDA (NHS-diazirine/succinimidyl 4,4′-azipentanoate), sulfo-SDA, LC-SDA (NHS-LC-diazirine/succinimidyl 6-(4,4′-azipentanamido)hexanoate), sulfo-LC-SDA, SDAD (NHS-SS-diazirine/succinimidyl 2-((4,4′-azipentanamido)ethyl)1,3′-dithiopropionate), sulfo-SDAD, or a combination thereof.

Monocytes can be contacted with an in vivo crosslinker. The contacting can be for a pre-determined time. The contacting can make some or all of the cells apoptotic. The in vivo crosslinker can comprise BS3, DTSSP, sulfo-EGS, DSG, DSP, DSS, EGS, sulfo-SDA, sulfo-LC-SDA, sulfo-SDAD, SDA, LC-SDA, SDAD, NHS-ester diazirine, or any combination thereof.

In some cases, the monocytes are treated with a cellular damaging agent or an apoptosis inducer. In some cases, the cellular damaging agent induces apoptosis in some or all of the contacted cells. Non-limiting exemplary cellular damaging agents include doxorubicin, staurosporine, etoposide, comptothecin, paclitaxel, vinblastine, or any combination thereof. Non-limiting exemplary apoptosis inducers include marinopyrrole A, maritoclax, (E)-3,4,5,4′-tetramethoxystilbene, 17-(Allylamino)-17-demethoxygeldanamycin, 2,4,3′,5′-tetramethoxystilbene, 20HOA, 6,8-bis(benzylthio)-octanoic acid, AT 101, apoptolidin, FET 40 A, ara-G hydrate, aryl quin 1, BAD, BAM7, BAX activator molecule 7, BH3I-1, BID, BMS-906024, BV02, bendamustine, borrelidin, borrelidine, cyclopentanecarboxylic acid, NSC 216128, treponemycin, brassinin, brassinine, brefeldin A, ascotoxin, BFA, cyanein, decumbin, bufalin, CCF642, CCT007093, CD437, CHM-1 hydrate, 2-(2-fluorophenyl)-6,7-methylenedioxy-2-4-quinolone hydrate, NSC 656158, CIL-102, CP-31398, dihydrochloride hydrate, carnal exin, 3-(Thiazol-2-yl)-1H-indole, carnal exine, carboxyatractyloside, cepharanthine, cepharanthine, cinnabarinic acid, cirsiliol, combretastatin A4, costunolide, DBeQ, DIM-C-pPhtBu, DMXAA, DPBQ, enniatin A1, enniatin A, enniatin B1, enniatin B, erastin, eupatorin, FADD, fluticasone propionate, fosbretabulin disodium, GO-201 trifluoroacetate, gambogic acid, HA 14-1, HMBA, hexaminolevulinate (HAL), IMB5046, IMS2186, ikarugamycin, imiquimod, iniparib, kurarinone, LLP-3, lipocalin-2, lometrexol, MI-4F, ML 210, ML291, mollugin, muristerone A, NA-17, NID-1, NPC26, NSC59984, Nap-FF, neocarzinostatin, nifetepimine, nitidine chloride, nutlin-3, nutlin-3a, PKF118-310, PRIMA-1, PRT4165, pemetrexed, penta-O-galloyl-P-D-glucose hydrate, phenoxodiol, prodigiosin (PG), psoralidin, pterostilbene, raltitrexed, raptinal, ridaifen-B, rifabutin, roslin 2, s-p-bromobenzylglutathione cyclopentyl diester, SJ-17255, SMBA1, STF-62247, suprafenacine, syrosingopine, talniflumate, taurolidine, temoporfin, temozolomide, tetrazanbigen, thaxtomin A, thiocolchicine, tirapazamine, UCD38B, UMI-77, undecylprodigiosin, VK3-OCH3, vacquinol-1, violacein, vosaroxin, zerumbone, gAcrp30, gAcrp30/adipolean, or any combination thereof. Cells contacted with a cellular damaging agent or an apoptosis inducer may subsequently be contacted with a fixative or cross-linking agent.

Monocytes can be made apoptotic by contacting the cells with a chemical (e.g., a fixative or cross-linking agent, a cellular damaging agent, or a combination thereof) for a predetermined amount of time. In some embodiments, the cells in the tolerizing vaccine or the preparatory regimen are made apoptotic by fixing for a predetermined amount time with the crosslinking agent (e.g., ECDI). In some cases, the predetermined amount of time is about 30 minutes, about 1 hour, about 2 hours, about 3 hours, about 4 hours, about 5 hours, about 6 hours, about 12 hours, about 18 hours, about 24 hours, about 36 hours, about 48 hours, about 60 hours, or about 72 hours. In some cases, the predetermined amount of time is less than an hour. In some cases, the predetermined time is at least about 1 minute, 5 minutes, 10 minutes, 20 minutes, 30 minutes, 40 minutes, 50 minutes, 60 minutes, 75, minutes, 90 minutes, 120 minutes, 150 minutes, 180 minutes, 210 minutes or 240 minutes. In some cases, the predetermined time is at most about 30 minutes, 40 minutes, 50 minutes, 60 minutes, 75, minutes, 90 minutes, 120 minutes, 150 minutes, 180 minutes, 210 minutes or 240 minutes. In some cases, the predetermined amount of time is about 1 minute to about 240 minutes, 1 minute to about 10 minutes, 10 minutes to about 240 minutes, about 10 minutes to about 180 minutes, about 10 minutes to about 120 minutes, about 10 minutes to about 90 minutes, about 10 minutes to about 60 minutes, about 10 minutes to about 30 minutes, about 30 minutes to about 240 minutes, about 30 minutes to about 180 minutes, about 30 minutes to about 120 minutes, about 30 minutes to about 90 minutes, about 30 minutes to about 60 minutes, about 50 minutes to about 240 minutes, about 50 minutes to about 180 minutes, about 50 minutes to about 120 minutes, about 50 minutes to about 90 minutes, about 50 minutes to about 60 minutes, about 10 minutes to about 20 minutes, about 20 minutes to about 30 minutes, about 30 minutes to about 40 minutes, about 40 minutes to about 50 minutes, about 50 minutes to about 60 minutes, about 60 minutes to about 70 minutes, about 70 minutes to about 80 minutes, about 80 minutes to about 90 minutes, about 90 minutes to about 100 minutes, about 100 minutes to about 110 minutes, about 110 minutes to about 120 minutes, about 10 minutes to about 30 minutes, about 30 minutes to about 50 minutes, about 50 minutes to about 70 minutes, about 70 minutes to about 90 minutes, about 90 minutes to about 110 minutes, about 110 minutes to about 130 minutes, about 130 minutes to about 150 minutes, about 150 minutes to about 170 minutes, about 170 minutes to about 190 minutes, about 190 minutes to about 210 minutes, about 210 minutes to about 240 minutes, up to about 30 minutes, about 30 minutes to about 60 minutes, about 60 minutes to about 90 minutes, about 90 minutes to about 120 minutes, or about 120 minutes to about 150 minutes.

The contacting can be at any temperature. In some cases, the contacting is performed on ice (e.g., at4° C.). In other cases, the contacting is performed at room temperature. In some cases, the contacting is performed at a temperature of at least about 0° C., 2° C., 4° C., 8° C., 15° C., 20° C., 25, 30° C., 35° C., or 37° C. In some cases, the contacting is performed at a temperature of at most about 4° C., 8° C., 15° C., 20° C., 25, 30° C., 35° C., 37° C., or 40° C. In some cases, the contacting is performed at a temperature of about 0° C. to about 37° C., about 0° C. to about 25° C., about 0° C. to about 15° C., about 0° C. to about 10° C., about 0° C. to about 8° C., about 0° C. to about 6° C., about 0° C. to about 4° C., about 0° C. to about 2° C., about 2° C. to about 10° C., about 2° C. to about 8° C., about 2° C. to about 6° C., about 4° C. to about 25° C., about 4° C. to about 10° C., about 15° C. to about 37° C., about 15° C. to about 25° C., about 20° C. to about 40° C., about 20° C. to about 37° C., or about 20° C. to about 30° C.

Monocytes can aggregate as a result of the method of making some or all of the cells apoptotic. For example, cells can aggregate after contacting with a chemical, such as a fixative or crosslinking agent. The predetermined amount of time that the cells are contacted with the chemical can be selected to minimize the amount of aggregation. In some cases, aggregates can be removed, for example, by washing and/or filtration.

In some cases, aggregated monocytes can comprise from or from about 0.01 to 10 aggregates, per ml. For example, the tolerizing vaccine or preparatory regimen can comprise from or from about 0.01 to 1, 0.1 to 1, 0.25 to 1, 0.5 to 1, 1 to 5; or 1 to 10 aggregate per pl. The tolerizing vaccine or preparatory regimen can comprise less than about 0.1, 0.5, 0.75, 1, 5, or 10 aggregates per mL.

In some cases, aggregated monocytes can comprise less than 5 aggregates per mL. For example, the tolerizing vaccine or preparatory regimen can comprise less than about: 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.05, or 0.01 aggregates per mL.

In some case, aggregated monocytes comprise 1 or fewer aggregates per mL. For example, the tolerizing vaccine or preparatory regimen can comprise about 0.01, about 0.05, about 0.1, about 0.2, about 0.3, about 0.4, about 0.5 about 0.6, about 0.7, about 0.8, about 0.9, or about 1.0 aggregates per mL.

In some embodiments, aggregated monocytes can include from or from about 0.01% to 10%, e.g., from or from about 0.01% to 2%, necrotic cells. For example, aggregated monocytes can comprise from or from about 0.01% to 10%; 0.01% to 7.5%, 0.01% to 5%; 0.01% to 2.5%; or 0.01% to 1% necrotic cells. In some embodiments, aggregated monocytes can comprise at most about 0.01%, 0.1%, 1%, 2%, 3%, 4%, 5%, 6%, 7%, 8%, 9%, or 10% necrotic cells.

In some embodiments, the manufactured therapeutic composition is stable at a temperature below zero degrees Celsius for at least about one month. In some embodiments, the apoptotic population is formulated in a pharmaceutically acceptable buffer suitable for parenteral administration to a human subject in need thereof.

### Apoptotic Monocytes

In one embodiment, apoptotic monocytes provide long term tolerance to a cell, a tissue, or an organ. In one embodiment, the donor monocytes are fixed in a cross-linking agent. In one embodiment, the apoptotic monocytes are mammalian monocytes. In one embodiment, the apoptotic monocytes are porcine monocytes. In one embodiment, the apoptotic monocytes are human monocytes. In one embodiment, the apoptotic monocytes are from a cadaveric donor, a brain-dead donor, a non-heart beating donor, or a living donor. In one embodiment, the apoptotic monocytes are ex vivo expanded monocytes. In one embodiment, the apoptotic monocytes are isolated from a spleen, or peripheral blood. In one embodiment, the apoptotic monocytes comprise B-lymphocytes. In one embodiment, the apoptotic monocytes comprise cells that have been differentiated from stem cells or induced pluripotent stem cells ex vivo. In some embodiments, the stem cells are derived from the donor of said transplant cell, tissue, or organ. In one embodiment, the apoptotic monocytes and the recipient are matched for at least one of MHC class IA allele, MHC class IB allele, MHC class II DR allele, MHC class IIDQ allele, or MHC class II DP allele. In one embodiment, the apoptotic monocytes and the transplant are matched for at least one of MHC class IA allele, MHC class IB allele, MHC class II DR allele, MHC class II DQ allele, or MHC class II DP allele. In one embodiment, the apoptotic monocytes and the transplant are haploidentical. the apoptotic monocytes are from the donor of the transplant.

1. Apoptosis Detection

Many commercial assays are available to detect apoptosis, such as assays to detect caspase activity, as caspases are activated during apoptosis. The assay can comprise detecting activation of the caspase, such as detecting zymogen processing of the one or more caspases, or detection of caspase function. Examples of such assays include, but are not limited to, PhiPhiLux® (Oncolmmunin, Inc.), Caspase 3 Activity Assay (Roche), Homogeneous Caspases Assay (Roche Applied Science), Caspase-Glo™ Assays (Promega), Apo-ONE® Homogeneous Caspase-3/7 Assay (Promega), CaspACE™ Assay System, Colorimetric (Promega), CaspACE™ Assay System, Fluorometric (Promega), EnzChek® Caspase-3 Assay Kit #1 (Invitrogen), Image-iT™ LIVE Green Caspase-3 and -7 Detection Kit (Invitrogen), Active Caspase-3 Detection Kits (Stratagene), Caspase-mediated Apoptosis Products (BioVision), and CasPASE™ Apoptosis Assay Kit (Genotech).

During fragmentation of the nucleus, endonuclease activation leaves short DNA fragments, which are often regularly spaced in size. These give a characteristic “laddered” appearance in electrophoresis, and this DNA laddering can be used to identify apoptosis. Many commercial assays available for detecting apoptosis is based on detecting this DNA fragmentation, such as with terminal deoxynucleotidyl transferase dUTP nick end labeling (TUNEL) or other types of DNA fragmentation assays. Examples of such assays include, but are not limited to, Apoptotic DNA Ladder Kit (Roche), Cellular DNA Fragmentation ELISA (Roche), Cell Death Detection ELISAPLUS (Roche), In Situ Cell Death Detection Kit (Roche), DeadEnd™ Fluorometric TUNEL System (Promega), DeadEnd™ Colorimetric TUNEI. System (Promega), APO-BrdU™ TUNEL Assay Kit (Invitrogen), TUNEL Apoptosis Detection Kit (Upstate), Apoptosis Mebstain Kit (Beckman Coulter), Nuclear-mediated Apoptosis Kits (BioVision), and Apoptotic DNA Ladder Kit (Genotech).

Another assay for apoptosis is the assay for detecting annexin V. Annexin V binds to phosphatidylserine (PS). Dying cells that undergo the final stages of apoptosis display phagocytotic molecules, such as PS on their cell surface. PS is normally found on the cytosolic surface of the plasma membrane, but is redistributed during apoptosis to the extracellular surface by a hypothetical protein. This allows PS to be indirectly detected by annexin V staining. Such commercially available assays include, but are not limited to, Annexin V, Alexa Fluor® 350 conjugate (Invitrogen), Rhodamine 110, bis-(L-aspartic acid amide), trifluoroacetic acid salt (Invitrogen), Annexin V, Alexa Fluor® 488 (Cambrex), and Annexin V Apoptosis Kits (BioVision).

Other assays for apoptosis can be for detecting of apoptotic markers, such as for Poly(ADP-ribose) polymerase (PARP), which is a nuclear enzyme involved in DNA repair. In many cell types, an early event during apoptosis is the proteolytic cleavage of PARP by a caspase. Thus, detecting of PARP, such as with anti-PARP, such as commercially available antibodies including, but not limited to anti-PARP from Roche, can be used for Western blot detection of the resulting proteolytic PARP fragments in extracts from early apoptotic cells. Another example is the detecting of cytokeratins. Cytokeratins, in particular cytokeratin 18, are subjected to proteolytic cleavage during the early stages of apoptosis. An antibody to detect one or more cytokeratins, such as the monoclonal antibody M30 CytoDEATH, which recognizes a specific caspase-cleavage site within cytokeratin 18 that is not detectable in the native CK18 of normal cells, can be used for detection of apoptosis.

The removal of dead cells, such as via apoptotic bodies, can be performed by an antigen presenting cell (APC). The APC can be a phagocytic cell or phagocyte. For example, the APC or phagocyte can be a macrophage. The macrophage can be identified by specific expression of one or more of the following markers, such as, but not limited to CD14, CD11b, P4/80 (mice)/EMR1 (human), Lysozyme M, MAC-1/MAC-3, and CD68. Identification can be by any means known in the art, such as by flow cytometry or immunohistochemical staining.

The apoptotic or monocyte can exhibit one or more molecules or markers that mark the apoptotic cell for phagocytosis by cells possessing the appropriate receptors, such as an APC or macrophage. Without being bound by any particular theory, upon recognition, the phagocyte typically reorganizes its cytoskeleton for engulfment of the apoptotic cell, thereby removing the dying cell, which is believed to occur in an orderly manner without eliciting an inflammatory response.

After digestion, a macrophage can present the antigen of the apoptotic cell to the corresponding helper T cell. The presentation can be performed by integrating the antigen into the cell membrane of the macrophage and displaying the antigen attached to an MHC class II molecule, which indicates to other white blood cells that the macrophage is not a pathogen, despite having antigens on its surface. In some embodiments, an apoptotic cell picked up by an antigen presenting cell, such as a host antigen presenting cell in the spleen, can induce tolerance. This presentation of the antigen to host T-cells in a non-immunogenic fashion can lead to direct induction of anergy.

2. Apoptosis Signaling Molecules

In some embodiments, the compositions of the present disclosure contain an apoptosis signaling molecule. The apoptotic signaling molecules serve allow a cell to be perceived as an apoptotic cell by antigen presenting cells of the host, such as cells of the host reticuloendothelial system. This allows presentation of the associated peptide epitopes in a tolerance-inducing manner. Without being bound by theory, this is presumed to prevent the upregulation of molecules involved in immune cell stimulation, such as MHC class I/II, and costimulatory molecules. These apoptosis signaling molecules may also serve as phagocytic markers. For example, apoptosis signaling molecules suitable for the present disclosure have been described in US Pat App No. 20050113297, which is hereby incorporated by reference in its entirety. Molecules suitable for the present disclosure include molecules that target phagocytes, which include macrophages, dendritic cells, monocytes and neutrophils.

Molecules suitable as apoptotic signaling molecules act to enhance tolerance of the associated peptides. Additionally, a carrier bound to an apoptotic signaling molecule can be bound by Clq in apoptotic cell recognition (Paidassi et al., (2008) J. Immunol. 180:2329-2338). For example, molecules that may be useful as apoptotic signaling molecules include phosphatidyl serine, annexin-1, annexin-5, milk fat globule-EGF-factor 8 (MFG-E8), or the family of thrombospondins.

Thrombospondins are a family of extracellular proteins that participate in cell-to-cell and cell-to-matrix communication. They regulate cellular phenotype during tissue genesis and repair. In addition, thrombospondin-1 (TSP-1) is expressed on apoptotic cells and is involved in their recognition by macrophages. Thrombospondin-1 is therefore another phagocytic marker that can be used to enhance phagocytosis in accordance with the disclosure. Macrophages recognize TSP-1 on apoptotic cells via the CD36 molecule, which is present on the surface of macrophages and may also be present on apoptotic cells. While not wishing to be bound by any theory, it is possible that CD36/TSP1 complex on the surface of an apoptotic cell may form a ligand bridging the cell to a complex consisting of alpha(v)beta 3/CD36/TSP1 on macrophages. It is possible that binding of TSP-1 to CD36 is mediated by interaction of the TSR-1 domain of TSP-1 with a conserved domain called CLESH-1 in CD36. In certain embodiments of the disclosure phagocytosis is enhanced by increasing the level or density of TSP-1, CD36, or a TSP-1/CD36 complex on the surface of a cell or molecule, e.g., by delivering the TSP-1, CD36, or TSP-1/CD36 complex to the cell. In certain embodiments of the disclosure a TSP-1/CLESH domain complex is delivered to the cell.

Alternatively or additionally, the phagocytic marker may comprise a molecule (e.g., MFG-E8, b2-glycoprotein, Phosphatidylserine, etc.) that serves as a bridging agent between macrophages and their targets, or a portion of such a molecule. Such markers may, for example, facilitate recognition of phosphatidyl serine by macrophages or be independently recognized. Other markers that are also known to enhance phagocytosis include protein S, the growth arrest specific gene product GAS-6, and various complement components including, but not limited to, factor B, Clq, and C3. As mentioned above, MFG-E8 is a secreted glycoprotein, which is produced by stimulated macrophages and binds specifically to apoptotic cells by recognizing aminophospholipids such as phosphatidylserine (PS). MFG-E8, when engaged by phospholipids, binds to cells via its RGD (arginine-glycine-aspartate) motif and binds particularly strongly to cells expressing alpha(v)beta(3) integrin, such as macrophages. At least two splice variants of MFG-E8 are known, of which the L variant is believed to be active for stimulating phagocytosis. In certain embodiments of the disclosure the phagocytic marker comprises the L splice variant of MFG-E8 (MFG-E8-L). In certain embodiments of the disclosure the phagocytic marker comprises an N-terminal domain of MFG-E8.

Annexin I is another phagocytic marker that may be used according to the present disclosure. Briefly, the 37 kDa protein annexin I (Anx-1; lipocortin 1) is a glucocorticoid-regulated protein that has been implicated in the regulation of phagocytosis, cell signaling and proliferation, and is postulated to be a mediator of glucocorticoid action in inflammation and in the control of anterior pituitary hormone release. Annexin I expression is elevated in apoptotic cells and appears to play a role in bridging phosphatidylserine on apoptotic cells to phagocytes and to enhancing recognition of apoptotic cells by phagocytes such as macrophages. While not wishing to be bound by any theory, it is possible that the phosphatidylserine receptor on macrophages recognizes either annexin I or a complex containing annexin I and PS, or that annexin I facilitates recognition by aggregating PS into clusters. Additionally, other DC targeting studies use conjugated targeting ligands such as anti-Dec-205 and anti-CD11c to increase DC specificity.

In some embodiments, the apoptotic signaling molecule may be conjugated to the antigen-specific peptide. In some instances, the apoptotic signaling molecule and antigen-specific peptide are conjugated by the creation of a fusion protein. As used herein, a “fusion protein” refers to a protein formed by the fusion of at least one antigen-specific peptide (or a fragment or a variant thereof) to at least one molecule of an apoptotic signaling molecule (or a fragment or a variant thereof). For the creation of fusion proteins, the terms “fusion protein,” “fusion peptide,” “fusion polypeptide,” and “chimeric peptide” are used interchangeably. Suitable fragments of the antigen-specific peptide include any fragment of the full-length peptide that retains the function of generating the desired antigen-specific tolerance function of the present disclosure. Suitable fragments of the apoptotic signaling molecules include any fragment of the full-length peptide that retains the function of generating an apoptotic signal. The present application is also directed to proteins containing polypeptides at least 80%, 85%, 90%, 95%, 96%, 97%, 98% or 99% identical to the reference polypeptide sequence (e.g., the antigen-specific peptide or apoptotic signaling molecule or the fusion protein thereof) set forth herein, or fragments thereof. Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide. As used herein, “variant”, refers to an antigen-specific peptide, apoptotic signaling molecule or fusion protein thereof differing in sequence from an antigen-specific peptide, apoptotic signaling molecule or fusion protein thereof of the disclosure, respectively, but retaining at least one functional and/or therapeutic property thereof (e.g., trigger tolerance in an immune system or produce an apoptotic signal). The present disclosure is also directed to proteins which comprise, or alternatively consist of, an amino acid sequence which is at least 80%, 85%, 90%, 95%, 96%, 97%, 98%, 99% or 100%, identical to, for example, the amino acid sequence of an antigen-specific peptide, apoptotic signaling molecule or fusion protein thereof of the disclosure.

The fusion protein may be created by various means. One means is by genetic fusion (i.e. the fusion protein is generated by translation of a nucleic acid sequence in which a polynucleotide encoding all or a portion or a variant of an antigen-specific peptide in joined in frame to a polynucleotide encoding all or a portion or a variant of an apoptotic signaling molecule. The two proteins may be fused either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. This term also refers to conservatively modified variants, polymorphic variants, alleles, mutants, subsequences, and interspecies homologs of the antigens that make up the fusion protein. The fusion protein may also be created by chemical conjugation. Protocols for generation of fusion polypeptides are well known in the art, and include various recombinant means and DNA synthesizers. Alternatively, the apoptotic signaling molecule and antigen-specific peptide fusion protein can also be easily created using PCR amplification and anchor primers that give rise to complementary overhangs between two consecutive gene fragments that can subsequently be annealed and reamplified to generate a chimeric gene sequence. For example, an apoptotic signaling molecule can be fused in-frame with an antigen-specific peptide. In the present disclosure, either the apoptotic signaling molecule or antigen-specific peptide may be the N-terminal portion of the fusion protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et. al., Gene 40:39-46 (1985); Murphy et al., Proc. Natl. Acad. Sci. USA 83:8258-8262 (1986); U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

### Inducing Tolerance for Autoimmune and Allergic Diseases

Compositions, methods, kits, and systems provided herein can be utilized to prevent and/or treat an autoimmune disorder. The term “autoimmune disorder”, “autoimmune disease”, “autoimmune condition”, and their grammatical equivalents as used herein can be used interchangeably.

The engineered monocytes as described herein comprise vesicles containing antigens (e.g., autoantigens, autoantigenic peptides, apoptotic cellular carriers) to induce antigen-specific T and B cell tolerance for treatment of an autoimmune condition.

Non-limiting examples of autoimmune disorders include inflammation, antiphospholipid syndrome, systemic lupus erythematosus, rheumatoid arthritis, autoimmune vasculitis, celiac disease, autoimmune thyroiditis, post-transfusion immunization, maternal-fetal incompatibility, transfusion reactions, immunological deficiency such IgA deficiency, common variable immunodeficiency, drug-induced lupus, diabetes mellitus, Type I diabetes, Type II diabetes, juvenile onset diabetes, juvenile rheumatoid arthritis, psoriatic arthritis, multiple sclerosis, immunodeficiency, allergies, asthma, psoriasis, atopic dermatitis, allergic contact dermatitis, chronic skin diseases, amyotrophic lateral sclerosis, chemotherapy-induced injury, graft-vs-host diseases, bone marrow transplant rejection, Ankylosing spondylitis, atopic eczema, Pemphigus, Behcet's disease, chronic fatigue syndrome fibromyalgia, chemotherapy-induced injury, myasthenia gravis, glomerulonephritis, allergic retinitis, systemic sclerosis, subacute cutaneous lupus erythematosus, cutaneous lupus erythematosus including chilblain lupus erythematosus, Sjogren's syndrome, autoimmune nephritis, autoimmune vasculitis, autoimmune hepatitis, autoimmune carditis, autoimmune encephalitis, autoimmune mediated hematological diseases, lc-SSc (limited cutaneous form of scleroderma), dc-SSc (diffused cutaneous form of scleroderma), autoimmune thyroiditis (AT), Grave's disease (GD), myasthenia gravis, multiple sclerosis (MS), ankylosing spondylitis transplant rejection, immune aging, rheumatic/autoimmune diseases, mixed connective tissue disease, spondyloarthropathy, psoriasis, psoriatic arthritis, myositis, scleroderma, dermatomyositis, autoimmune vasculitis, mixed connective tissue disease, idiopathic thrombocytopenic purpura, Crohn's disease, human adjuvant disease, osteoarthritis, juvenile chronic arthritis, a spondyloarthropathy, an idiopathic inflammatory myopathy, systemic vasculitis, sarcoidosis, autoimmune hemolytic anemia, autoimmune thrombocytopenia, thyroiditis, immune-mediated renal disease, a demyelinating disease of the central or peripheral nervous system, idiopathic demyelinating polyneuropathy, Guillain-Barre syndrome, a chronic inflammatory demyelinating polyneuropathy, a hepatobiliary disease, infectious or autoimmune chronic active hepatitis, primary biliary cirrhosis, granulomatous hepatitis, sclerosing cholangitis, inflammatory bowel disease (including Crohn's disease (CD) and ulcerative colitis (UC)), gluten-sensitive enteropathy, Whipple's disease, an autoimmune or immune-mediated skin disease, a bullous skin disease, erythema multiforme, allergic rhinitis, atopic dermatitis, food hypersensitivity, urticaria, an immunologic disease of the lung, eosinophilic pneumonias, idiopathic pulmonary fibrosis, hypersensitivity pneumonitis, a transplantation associated disease, graft rejection or graft-versus-host-disease, psoriatic arthritis, psoriasis, dermatitis, polymyositis/dermatomyositis, toxic epidermal necrolysis, systemic scleroderma and sclerosis, responses associated with inflammatory bowel disease, Crohn's disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Diamond Blackfan anemia, immune hemolytic anemia including autoimmune hemolytic anemia (AIHA), pernicious anemia, pure red cell aplasia (PRC A), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, mysathenia gravis, antigen-antibody complex mediated diseases, anti-glomerular basement membrane disease, anti-phospholipid antibody syndrome, allergic neuritis, Bechet disease, Castleman's syndrome, Goodpasture's syndrome, Lambert-Eaton Myasthenic Syndrome, Reynaud's syndrome, Sjorgen's syndrome, Stevens-Johnson syndrome, pemphigoid bullous, pemphigus, autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic thrombocytopenic purpura (TTP), autoimmune thrombocytopenia, autoimmune disease of the testis and ovary including autoimmune orchitis and oophoritis, primary hypothyroidism, autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Sheehan's syndrome, autoimmune hepatitis, lymphoid interstitial pneumonitis (HIV), bronchiolitis obliterans (non-transplant) vs NSIP, Guillain-Barre' Syndrome, large vessel vasculitis (including polymyalgia rheumatica and giant cell (Takayasu's) arteritis), medium vessel vasculitis (including Kawasaki's disease and polyarteritis nodosa), ankylosing spondylitis, Berger's disease (IgA nephropathy), rapidly progressive glomerulonephritis, primary biliary cirrhosis, Celiac sprue (gluten enteropathy), cryoglobulinemia, and amyotrophic lateral sclerosis (ALS). In some cases, the autoimmune disease is SLE, rheumatoid arthritis, or celiac's disease.

Some exemplary autoimmune diseases and target antigens are disclosed in Table 2.

TABLE 2

| | Specific Disease | Target Antigen |
|---|---|---|
| Autoimmune | Celiac Disease | Gliadin, Barley, Hordein |
| diseases | Multiple Sclerosis | MBP13-32, MBP83-99, |
| | | MBP111-129, MBP146- |
| | | 170, MOG1-20, MOG35- |
| | | 55, and PLP139-15 |
| | Type 1 diabetes | Insuling, Pro-insulin, |
| | | IGRP, etc |
| | Myasthenia gravis | |
| Transplantation, | Transplantation | Allogenic transplanted cells |
| cell therapy and | GVHD | Allogenic transplanted cells |
| tissue regeneration | Allogeneic CART | Allogenic transplanted cells |
| therapies | cell therapy | |
| Allergies | Milk | Ceasin etc |
| | Peanut | |
| | Cedar Pollen | JCP, etc |
| Enzyme replacement | Fabrys, Hemophilia, | Enzymes, Factor VIII, etc |
| | etc | |
| Anti-drug-antibody | Humira, etc | Prevent neutralization |
| | | of the drug |

### Methods of Treatment

Provided herein is a method of treating an immune-mediated disease or condition in a subject by administering to the subject a pharmaceutical composition comprising a therapeutically effective amount of the exogenously modified population of monocytes. The immune-mediated disease or condition is characterized by an aberrant immune response to an antigen, wherein treating the subject reduces or ameliorates the aberrant immune response.

In some embodiments, the immune-mediated disease or condition is selected from the group consisting of multiple sclerosis (MS), rheumatoid arthritis, systemic lupus erythematosus (lupus), inflammatory bowel disease (IBD), ulcerative colitis, Crohn's disease, type 1 diabetes mellitus, Guillain-Barre syndrome, chronic inflammatory demyelinating polyneuropathy, psoriasis, Graves' disease, Hashimoto's thyroiditis, myasthenia gravis, Addison's disease, Sjögren's syndrome, pernicious anemia, celiac disease and vasculitis.

In some embodiments, the immune-mediated disease or condition is selected from the group consisting of eczema (atopic dermatitis), asthma.

In some embodiments, the immune-mediated disease or condition is selected from the group consisting of a food allergy.

In some embodiments, the immune-mediated disease or condition is transplant rejection.

In some embodiments, the immune-mediated disease or condition is a lysosomal storage disease.

Provided herein is a method of treating or inducing immune tolerance to an autoantigenic peptide in a human subject with an immune-mediated disease or condition, the method comprising administering to the human subject the pharmaceutical composition of any one of embodiments described herein, wherein the pharmaceutical composition comprises a therapeutically effective amount of the exogenously modified population of monocytes. In some embodiments, the immune-mediated disease or condition is an autoimmune disease, an autoinflammatory disease or condition, an allergic condition, a host-versus graft rejection disease.

In some embodiments, administering comprises intravenous administration. In some embodiments, administration of the pharmaceutical composition elicits an innate immune response in the subject. In some embodiments, monocytes of the exogenously modified population of monocytes migrate to a splenic marginal zone sinus of the subject after administration of the pharmaceutical composition. In some embodiments, antigen presenting cells (APCs) of the subject engulf or phagocytose monocytes of the exogenously modified population of monocytes after administration of the pharmaceutical composition. In some embodiments, the monocytes of the exogenously modified population of monocytes undergo scavenger receptor-mediated uptake by APCs of the subject after administration of the pharmaceutical composition. In some embodiments, the APCs of the subject present the autoantigenic peptide in complex with an MHC protein after administration of the pharmaceutical composition.

In some embodiments, the exogenously modified population of monocytes do not present the autoantigenic peptide at the time of administration.

In some embodiments, administering comprises intravenous administration.

In some embodiments, administration of the pharmaceutical composition elicits an innate immune response in the subject.

In some embodiments, monocytes of the exogenously modified population of monocytes migrate to a splenic marginal zone sinus of the subject after administration of the pharmaceutical composition.

In some embodiments, antigen presenting cells (APCs) of the subject engulf or phagocytose monocytes of the exogenously modified population of monocytes after administration of the pharmaceutical composition. In some embodiments, the monocytes of the exogenously modified population of monocytes undergo scavenger receptor-mediated uptake by APCs of the subject after administration of the pharmaceutical composition. In some embodiments, the APCs of the subject present the autoantigenic peptide in complex with an MHC protein after administration of the pharmaceutical composition.

In some embodiments, the MHC protein is an MHC class II protein.

In some embodiments, the APCs of the subject secrete a cytokine or growth factor after administration of the pharmaceutical composition.

In some embodiments, the cytokine or the growth factor regulates co-stimulatory molecules.

In some embodiments, the cytokine or the growth factor is IL-10 and/or TGFbeta.

In some embodiments, the APCs of the subject increase expression a negative costimulatory molecule after administration of the pharmaceutical composition.

In some embodiments, the negative costimulatory molecule comprises PD-L1, CTLA-4 or any combination thereof.

In some embodiments, the APCs of the subject express IL-10 receptor.

In some embodiments, the APCs of the subject are selected from the group consisting of B cells, macrophages, dendritic cells and a combination thereof.

In some embodiments, the macrophages are marginal zone macrophages (MZMs).

In some embodiments, the effector T cells comprising a T cell receptor (TCR) specific to a peptide:MHC complex comprising the autoantigenic peptide undergo apoptosis after administration of the pharmaceutical composition. In some embodiments, the effector T cells comprising a TCR specific to a peptide:MHC complex comprising the autoantigenic peptide differentiate into anergic T cells after administration of the pharmaceutical composition.

In some embodiments, the regulatory T cells (Tregs) of the subject comprising a TCR specific to a peptide:MHC complex comprising the autoantigenic peptide are upregulated after administration of the pharmaceutical composition. In some embodiments, naive T cells of the subject differentiate into Tregs after administration of the pharmaceutical composition. In some embodiments, naive T cells of the subject comprising a TCR specific to a peptide:MHC complex comprising the autoantigenic peptide differentiate into anergic T cells after administration of the pharmaceutical composition. In some embodiments, the T cells are CD4+ T cells.

In some embodiments, the exogenously modified population of monocytes comprises exogenously modified monocytes that are phagocytosed, engulfed and/or recognized as apoptotic by APCs of the subject. In some embodiments, the method further comprises modifying a population of monocytes, thereby generating the exogenously modified population of monocytes. In some embodiments, modifying comprises introducing the recombinant polynucleotide into the population of monocytes. In some embodiments, introducing comprises electroporating, transfecting, nucleofecting or transducing. In some embodiments, modifying comprises culturing the population of monocytes ex vivo. In some embodiments, culturing comprises inducing maturation, differentiation or apoptosis of the population of monocytes. In some embodiments, modifying comprises contacting a population of monocytes with an agent that modifies the monocytes such that APCs of the human subject phagocytose or engulf exogenously modified monocytes of the exogenously modified population of monocytes.

In some embodiments, the population of monocytes is an allogeneic or autologous population of monocytes. In some embodiments, the method comprises obtaining, isolating or enriching a population of monocytes from a biological sample from a human subject. In some embodiments, the biological sample is a peripheral blood mononuclear cell (PBMC) sample or a leukapheresis. In some embodiments, the human subject has T cells with a TCR that is specific to a peptide:MHC complex comprising the autoantigenic peptide.

In some embodiments, the method further comprising administering an additional therapeutic to the subject.

In some embodiments, the additional therapeutic comprises an immunosuppressive agent.

In some embodiments, the pharmaceutical composition is administered more than once.

In some embodiments, the pharmaceutical composition is administered periodically at an interval of 2 months, 3 months, 4 months, 5 months, 6 months, 8 months, 10 months or 12 months.

In some embodiments, the human subject is positive for an HLA allele that specifically binds to the autoantigenic peptide.

In some embodiments, the method comprises prior to administering the pharmaceutical composition, selecting a human subject that is positive for an HLA allele that specifically binds to the autoantigenic peptide.

In some embodiments, the immune-mediated disease or condition is selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo and Vogt-Koyanagi-Harada Disease.

In some embodiments, the disease treated by the compositions and methods described herein is liver fibrosis. Fibrosis is the excessive accumulation of extracellular matrix that often occurs as a wound healing response to repeated or chronic tissue injury, and may lead to the disruption of organ architecture and loss of function. Although fibrosis was previously thought to be irreversible, recent evidence indicates that certain circumstances permit the resolution of fibrosis when the underlying causes of injury are eradicated. The mechanism of fibrosis resolution encompasses degradation of the fibrotic extracellular matrix as well as elimination of fibrogenic myofibroblasts through their adaptation of various cell fates, including apoptosis, senescence, dedifferentiation, and reprogramming. Here we have developed therapeutic approaches to alter the immune response that may identify result in therapeutic approaches for fibrosis.

A broad range of prevalent chronic diseases can give rise to fibrosis, including diabetes, hypertension, viral and nonviral hepatitis, heart failure and cardiomyopathy, idiopathic pulmonary disease, scleroderma, and cancer. Fibrosis resulting from cirrhosis other diseases can lead to failure of liver, lung, kidney, heart, or other vital organs as excessive ECM replaces and disrupts parenchymal tissue. Consequently, severe fibrosis is estimated to account for up to 45% of all deaths in the developed world. Current therapies for fibrosis are few and of limited efficacy. Therefore, there is an urgent need to understand how fibrosis may regress and to identify potential therapeutic approaches.

Primary barrier for using myeloid cell therapy/cellular programming to treat fibrotic diseases is the inability of these cells to recognize disease tissue and respond in a therapeutic fashion. By engineering myeloid cells with CAR's that promote disease tissue recognition and activity these inventions overcome these barriers. Additionally myeloid cells can be engineered and induced to promote phagocytosis and resolution of fibrosis. Additionally, myeloid cells can be programmed to address different stages of fibrosis. For example, specific constructs can be engineered to deliver the correct “signals” to reverse the disease and can be made to be stage specific.

### EXAMPLES

The present disclosure will be better understood with reference to the following examples. These examples are intended to representative of specific embodiments of the disclosure, and are not intended as limiting the scope of the disclosure.

### Example 1: Isolation, Preparation and Characterization of CD14+ Monocytes from a Subject

The enriched autologous monocyte fractions are isolated from a 10 L target blood volume leukapheresis. The leukapheresis product is analyzed for white blood cell count with a differential analysis of the cell types. The leukapheresis product is transferred to the Manufacturing Facility where it is sampled for comparison of the starting population and the elutriated fractions.

The leukapheresis product is then diluted to a final volume of 300 mL with 1× Phosphate Buffered Saline. The Elutra® Cell Separation System, a semi-automatic, closed centrifuge-based laboratory system that uses continuous counter-flow elutriation technology to separate cell products into multiple fractions. The system uses a single-use tubing. The diluted leukapheresis product is then plumbed to the using the piercing pin supplied with the TerumoBCT Elutra® Set. The total white blood cell and red blood cell count, supplied with the leukapheresis product, is entered into the Elutra® program and the process is initiated. The elutriation process separates the leukapheresis product into 5 fractions by washing a cell bed with Lonza (SartoriusStedim) BioWhittaker® HBSS (1×) Without Calcium, Magnesium, or Phenol Red containing Grifols Albumin (Human) USP Albutein® and separating the cell types by their size and density. The first fraction contains residual plasma, the second fraction contains red blood cells, the third fraction is enriched with T-Cells and the fourth and fifth fractions are enriched for monocytes. Fractions three, four, and five are all sampled, stained for monocytes using CD14 as a marker and T-Cells using CD3 as a marker and analyzed via flow cytometry.

Post elutriation, fractions four and five are isolated via centrifugation for 15 minutes at 300×g at 20° C. The cells are then resuspended in 25-50 mL of pre-warmed recovery media, consisting of Miltenyi TexMACS™ GMP media, Miltenyi MACS® GMP Recombinant Human MCSF, and Gemini Bio-Products GemCell™ Human AB Serum Heat Inactivated, in a Corning® 50 mL Mini Bioreactor. A sample is taken to determine viable cell concentration and is recorded and trended for overall processing efficiency. The fractions are placed in a humidified incubator controlled at 37° C. and 5% carbon dioxide for 45 to 180 minutes.

After the incubation time, the cells are isolated from the recovery media via centrifugation for 10 minutes at 300×g at 20° C. The cells are initially resuspended in MaxCyte Electroporation Buffer and a sample is taken for viable cell concentration determination. The cells are isolated again via centrifugation for 15 minutes at 300×g at 20° C. and resuspended at a calculated 1×108 viable cells per mL in MaxCyte Electroporation Buffer. The cells are then ready for the electroporation process.

### Cell Count and Purity:

Cell counts of total MNCs and isolated monocyte fractions are performed using a Sysmex XP-300 automated analyzer (Sysmex). Assessment of macrophage numbers is carried out by flow cytometry with TruCount tubes (Becton Dickinson) to determine absolute cell number, as the Sysmex consistently underestimated the number of macrophages. The purity of the separation is assessed using flow cytometry (FACSCanto II, BD Biosciences) with a panel of antibodies against human leukocytes (CD45-VioBlue, CD15-FITC, CD14-PE, CD16-APC), and product quality is assessed by determining the amount of neutrophil contamination (CD45int, CD15pos).

### Flow Cytometry Characterization:

Monocyte cell surface marker expression is analyzed using either a FACSCanto II (BD Biosciences) or MACSQuant 10 (Miltenyi) flow cytometer. Approximately 20,000 events are acquired for each sample. Cell surface expression of leukocyte markers in freshly isolated and day 7 matured cells is carried out by incubating cells with specific antibodies (final dilution 1:100-dilution may vary depending on the depending on antibody manufacturer and quality). Cells are incubated for 5 min with FcR blocking antibody (Miltenyi) then incubated at 4° C. for 20 min with labelling antibody cocktails. Cells are washed in PEA, and dead cell exclusion dye DRAQ7 (BioLegend) is added at 1:100. Cells are stained for a range of surface markers as follows: CD45-VioBlue, CD14-PE or CD14-PerCP-Vio700, CD163-FITC, CD169-PE and CD16-APC (all Miltenyi), CCR2-BV421, CD206-FITC, CXCR4-PE and CD115-APC (all BioLegend), and 25F9-APC and CD115-APC (eBioscience). Monocytes are gated to exclude debris, doublets and dead cells using forward and side scatter and DRAQ7 dead cell stain and analyzed using FlowJo software (Tree Star). From the initial detailed phenotyping, a panel is developed as a Release Criteria (CD45-VB/CD206-FITC/CD14-PE/25F9 APC/DRAQ7) that defines functional macrophage from monocytes. Macrophages are determined as having mean fluorescence intensity (MFI) five times higher than the level on day 0 monocytes for both 25F9 and CD206. A second panel assess other markers as part of an Extended Panel, composed of CCR2-BV421/CD163-FITC/CD169-PE/CD14-PerCP-Vio700/CD16-APC/DRAQ7), but is not used as part of the Release Criteria for the cell product.

### Example 2: mRNA Production and Electroporation

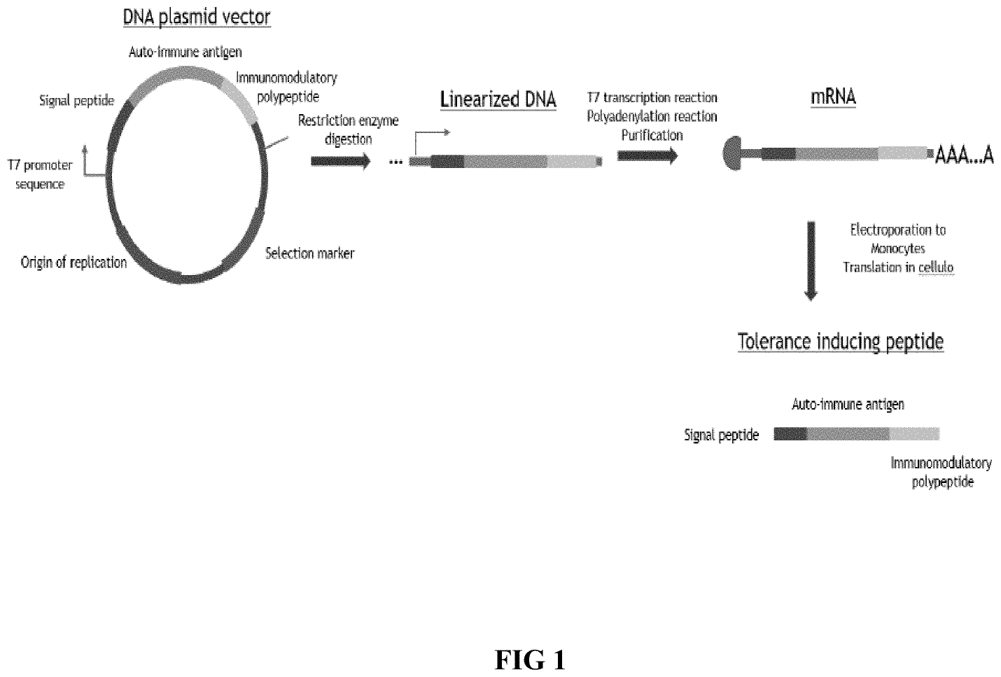

A recombinant nucleic acid, e.g., a plasmid (FIG. 1), encoding the antigen or a detectable protein such as GFP is constructed as follows: a signal peptide sequence that encodes for a localization signal is placed upstream of the coding sequence of the antigen or detectable protein. An enhancer such as LAMP-1 may be placed further downstream of the coding sequence. mRNA encoding the antigen or detectable protein is transcribed using a T7 polymerase (NEB) from the DNA plasmid and is tailed using a poly-A polymerase (NEB), treated with a DNAse enzyme to remove the plasmid template, and column purified with silica columns (NEB). mRNA may be further purified using ethanol precipitation and HPLC. Human monocytes are then electroporated with the mRNA as follows:

Monocytes are counted and washed once with MaxCyte electroporation buffer. Monocytes are resuspended in the appropriate electroporation volume (2-20 mL) at a density of 1×10⁸ cells/mL and mixed with purified mRNA. This mixture is placed in the MaxCyte electroporation cuvette, electroporated, and incubated in a 37 C incubator for 10 min. The cells are then taken out of the cuvette, which is washed once to remove residual cells, and placed in culture media (MCSF+TexMACS) to recover.

### Example 3: ECDI-Treatment of Modified Monocytes

2×10⁹ isolated engineered monocytes are suspended in 10-20 ml saline and 1 ml of 100 mg/ml of freshly prepared water-soluble 1-ethyl-3-(3-dimethylaminopropyl-)-carbodiimide (EDC) (Calbiochem, Darmstadt, Germany) is added to suspended monocytes. Following 1 h incubation shaking at 4° C., the cells are washed 2 times with 100 ml Citrate-Phosphate-Dextrose 12.6%/saline and finally 1×10⁹ cells are re-suspended in 100 ml of autologous plasma for injection to a subject. Cells are carefully checked for the absence of clumping.

### Example 4: Apoptosis Detection

Apoptosis is evaluated in two replicate experiments using the Early Apoptosis Detection kit (Kamiya Biochemical, Seattle, Wash.). After treatment of engineered monocytes with ECDI, viable cells are recovered by Ficoll-Paque separation, washed, and stained with Annexin V-FITC and propidium iodide according to manufacturer instructions. The cells are identified by staining of exposed phosphatidylserine with Annexin V-FITC but not propidium iodide, and necrotic cells were identified by Annexin V staining and uptake of propidium.

### Example 5: Immunomodulation by the Pharmaceutical Product (Experimental Multiple Sclerosis Model)

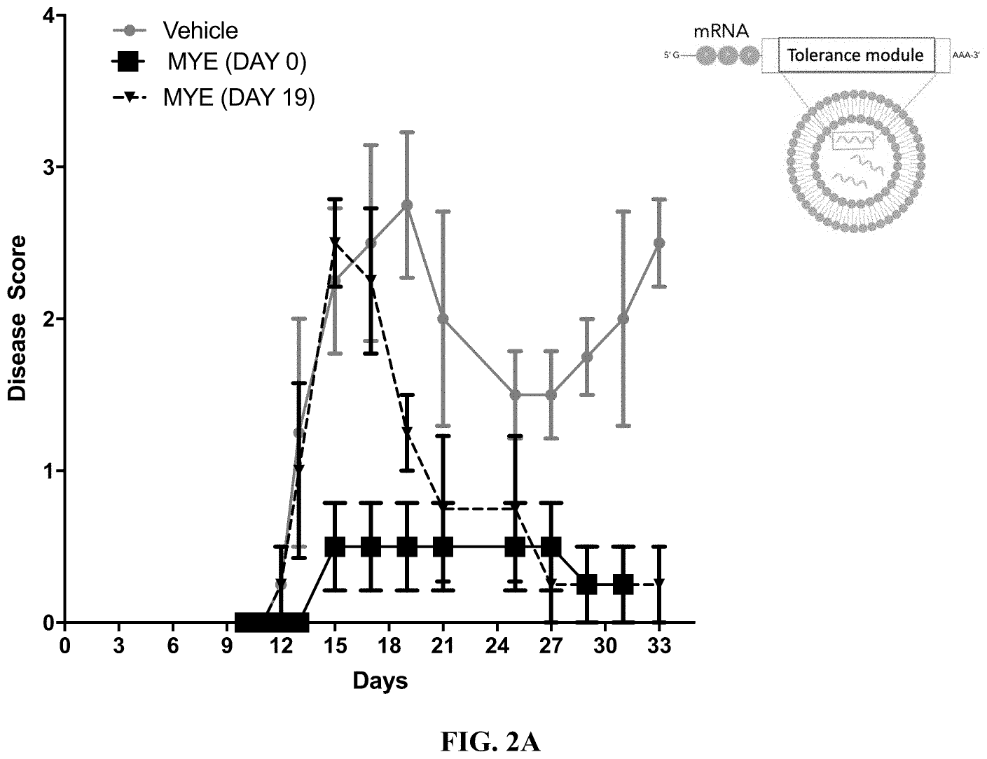

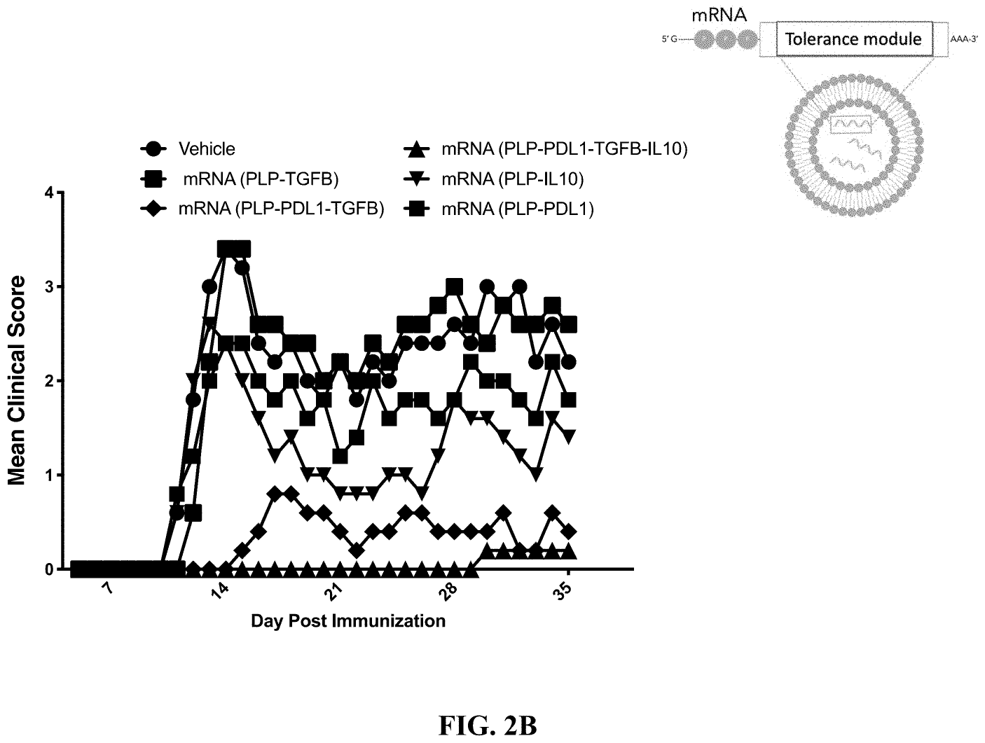

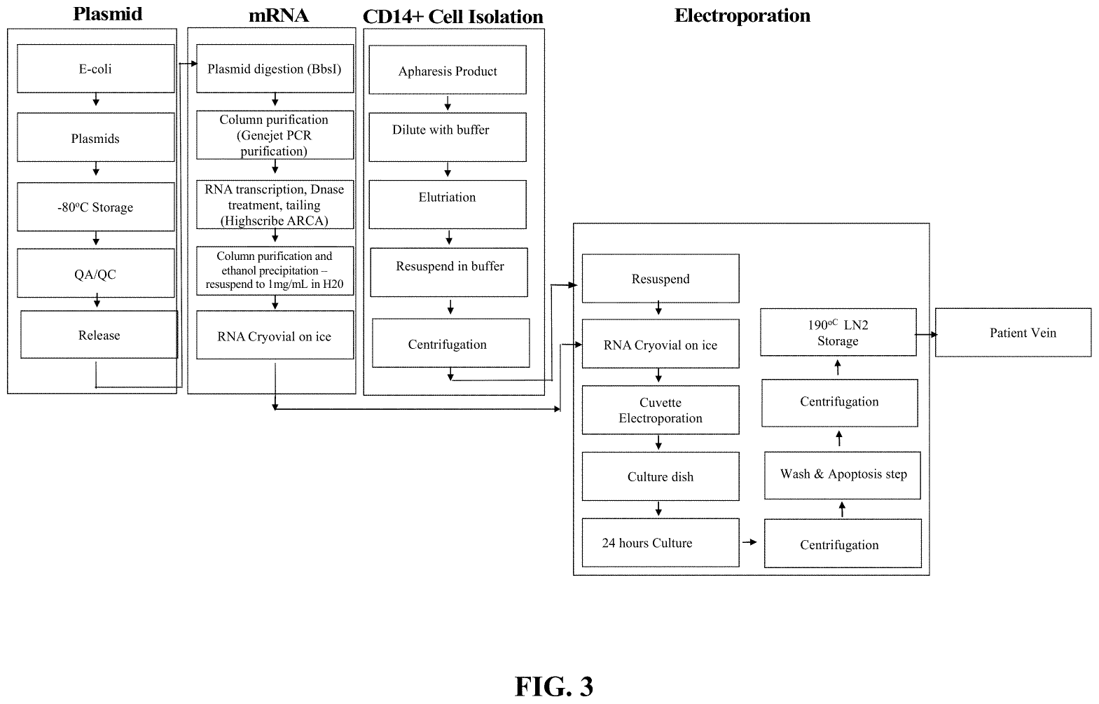

To test the effect of pharmaceutical composition, experimental autoimmune encephalitis EAE (mouse model of MS) is induced by subcutaneous injection of Proteolipid peptide (PLP, aa139-151) emulsified in CFA ion 30 mice. Mice are then randomized into 3 groups of ten mice. Group one received no treatment, Group 2 received 1×10⁷ MIT₁₃₉₋₁₅₁ cells at the time of immunization and Group 3 received 1×10⁷ MIT₁₃₉₋₁₅₁ cells at peak disease (e.g., day 17). Treatment with MIT cells results in prevention of disease (group 2 animals). In animals with ongoing disease, treatment results in amelioration of disease and the prevention of relapse. Together the data show MIT cells induce immune tolerance (FIGS. 2A, and 2B).

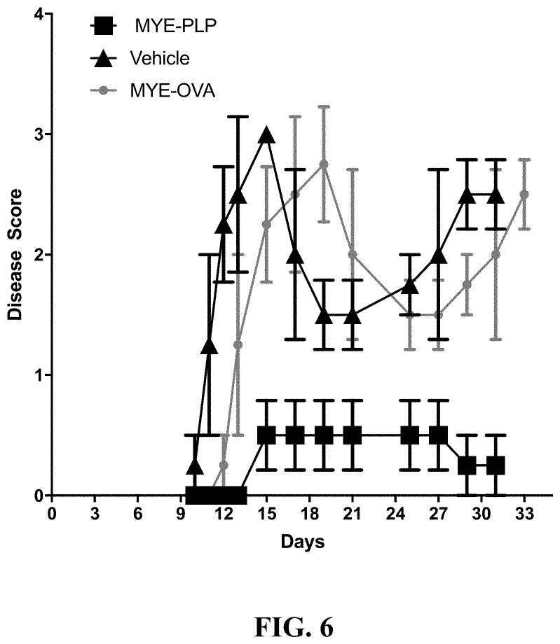

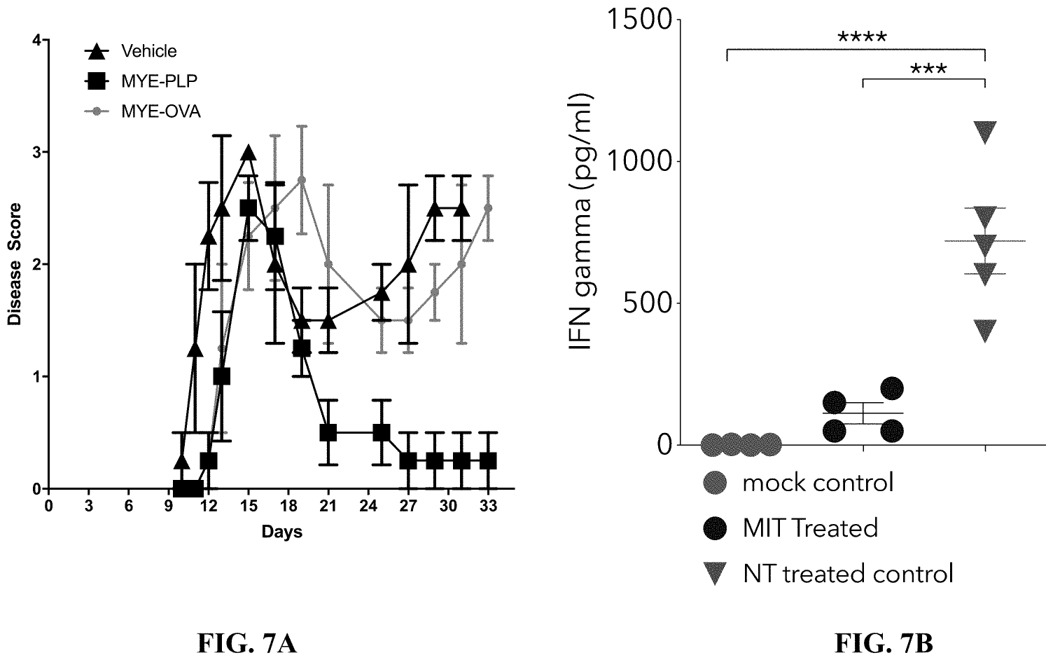

FIG. 6 and FIG. 7A show disease scores over the time-course comparing mice with vehicle administration (control), myeloid cells expressing OVA antigen (negative control) and myeloid cells expressing PLP administered from day 0 (FIG. 6) or at peak disease period (FIG. 7A). These results demonstrate that treatment with MIT cells results in prevention of disease (group 2 animals). In animals with ongoing disease (Group 3 animals), treatment resulted in amelioration of disease and the prevention of relapse. Together the data show MIT cells induce immune tolerance. B. Splenic cells were isolated from the treated mice in the study and evaluated for inflammatory potential, as determined by IFNγ secretion. As shown in FIG. 7B, cells from the non-transfected control set showed high IFN gamma expression, whereas the MIT treated cells had remarkable suppression of the inflammatory cytokine, indicating that an immunosuppression was induced in vivo that changed the behavior of the splenic cells.

### Example 6: Immunomodulation by the Pharmaceutical Product (Experimental Celiac Disease Model)

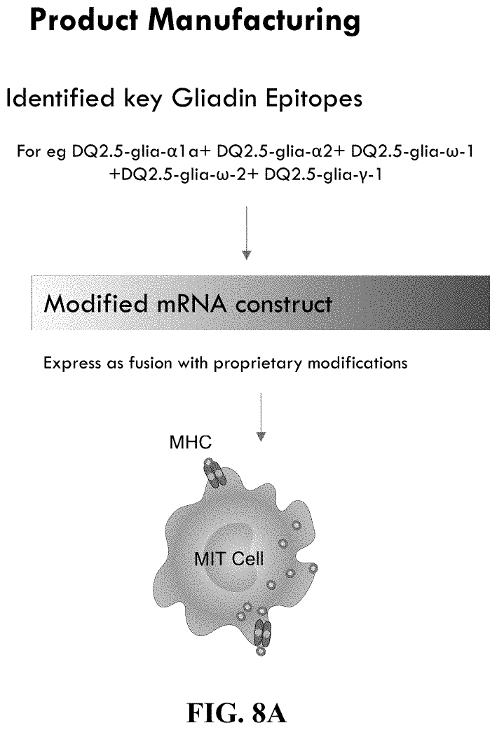

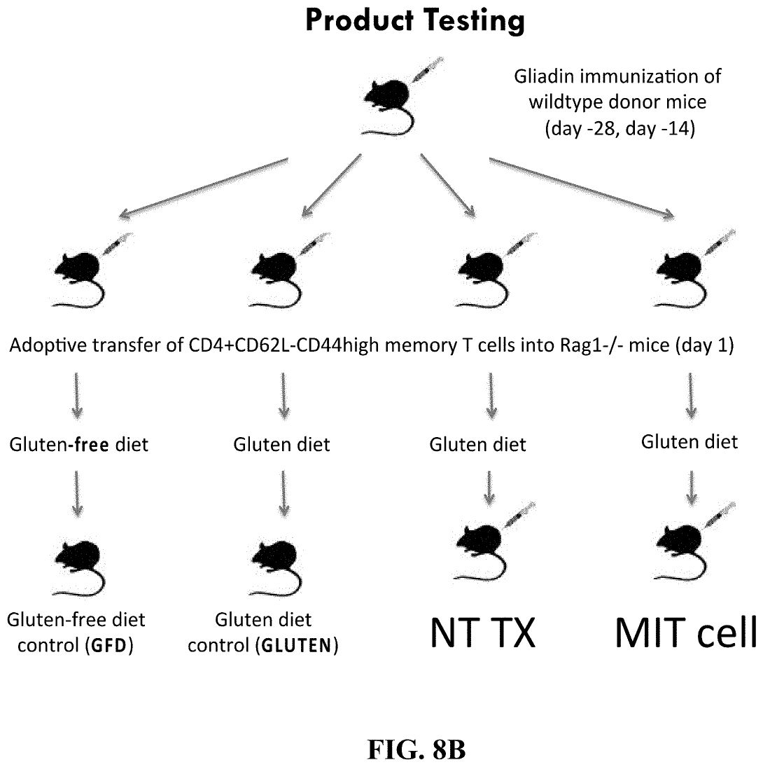

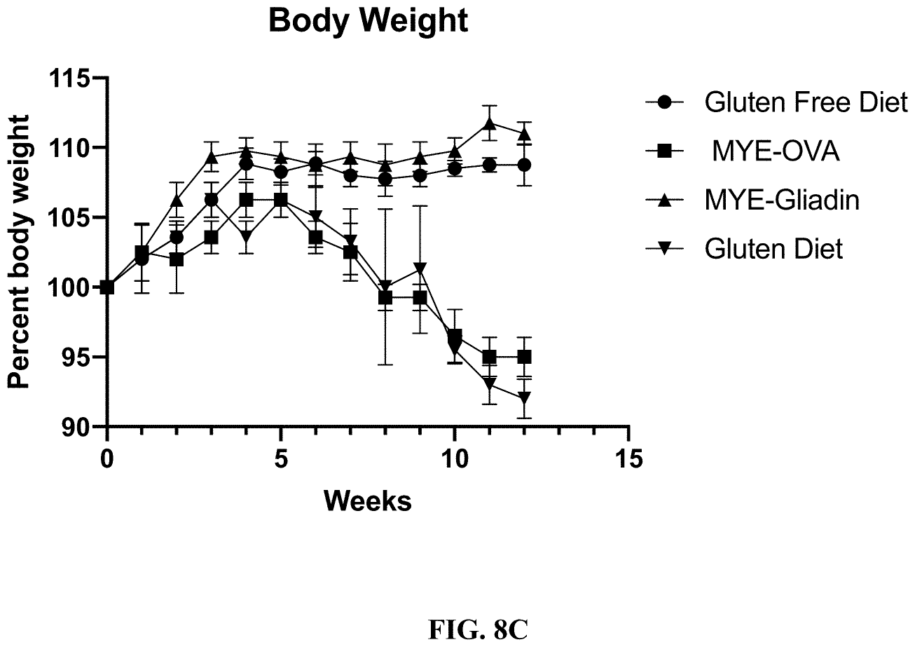

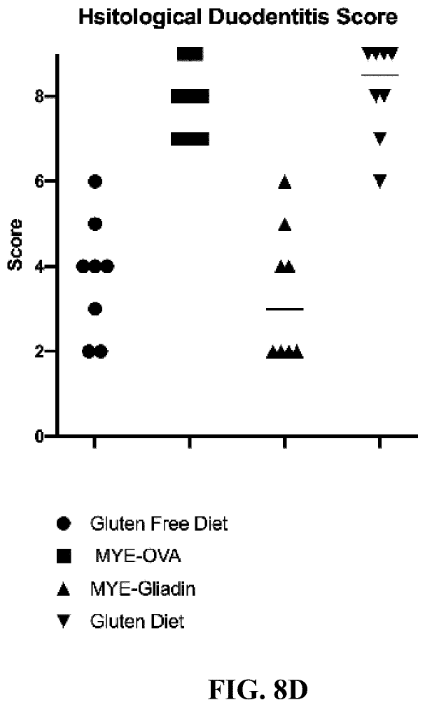

FIG. 8A and FIG. 8B discloses the experimental settings with gliadin epitopes, for example, DQ2.5-glia-α1a, DQ2.5-glia-α2, DQ2.5-glia-ω-1, DQ2.5-glia-ω-2+DQ2.5-glia-γ-1. Recombinant mRNA constructs are designed with modifications as discussed in the instant disclosure. mRNA expressing one or more gliadin epitopes are expressed in MIT cells by electroporation. Mouse model for celiac disease is generated by the method laid out in FIG. 8B. Body weights of mice from respective groups are monitored over time. Mice with gluten diet lose weight over time due to high T cell mediated autoimmune activity, in contrast to mice that are fed gluten free diet. Surprisingly, mice that are administered engineered and modified myeloid cells (MIT cells) expressing the gliadin epitopes and fed gluten diet, did not lose weight over time (FIG. 8C). Mice treated with MIT cells expressing OVA epitopes one the other hand lost weight similar to the diseased mice fed gluten diet. FIG. 8D shows histological scores for duodenitis, which again illustrate the protective effect of the engineered myeloid cells in the experimental disease model.

### Example 7: Modifications of mRNA

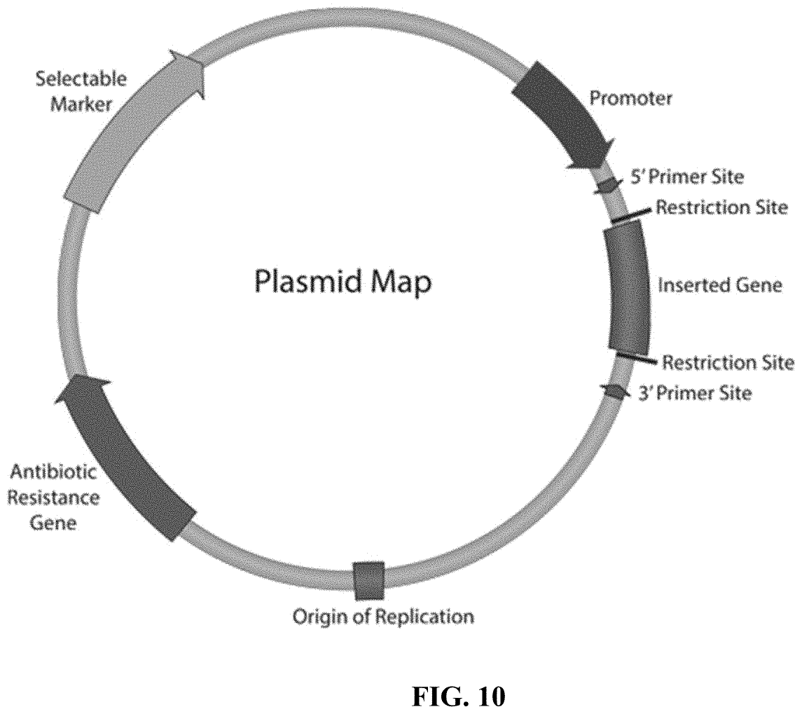

In this example, a non-exhaustive modifications of mRNA for increased stability and increased in vivo half-life is demonstrated. As shown in FIG. 9, the mRNA comprises a 5′-CAP, wherein the CAP can be modified to comprise ARCA, and modifications to reduce mRNA degradation, modifications to promote expression, known to one of skill in the art. Additional sequences encoding accessory proteins and helper peptides that assist HLA peptide loading can be incorporated for expression in the mRNA, and can be separated from other sequence (e.g. proteins, enzymes) by post-translational cleavage sites such as T2A. The order of these sequences as arranged on the mRNA can be reversed or arranged for most suitable expression of all the sequence components. mRNA can be in vitro transcribed from a vector encoding the recombinant sequence (FIG. 10), which comprise components for expansion and selection (e.g., promoter, antibiotic resistance gene and selection marker).

FIG. 11 exemplifies additional mRNA tolerogenic modules.

### Example 8: Engineered Myeloid Cells Reverse Liver Fibrosis in a Mouse Model

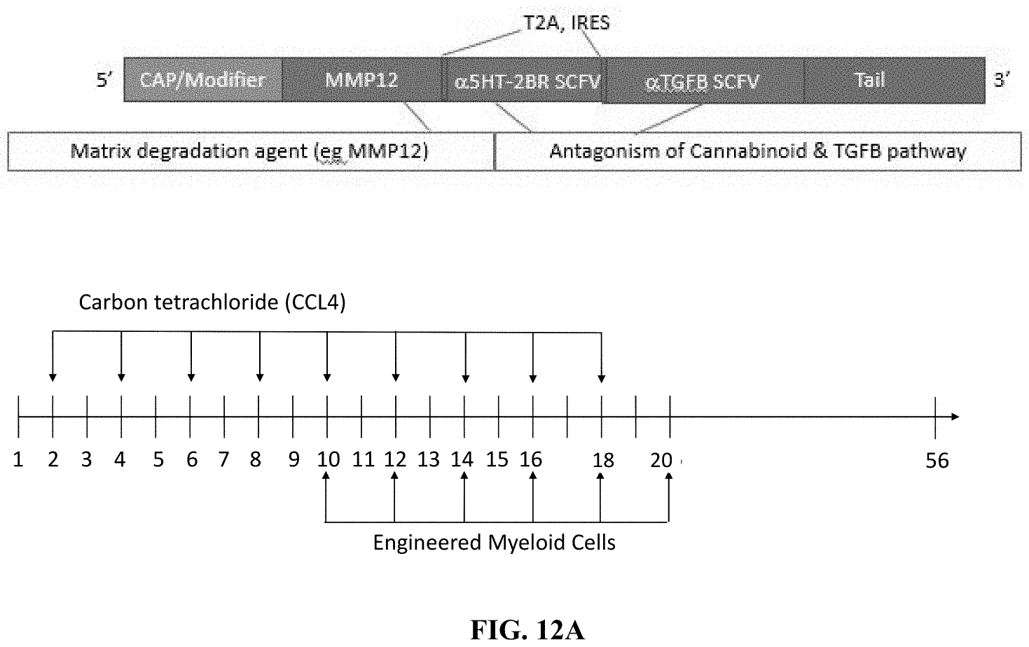

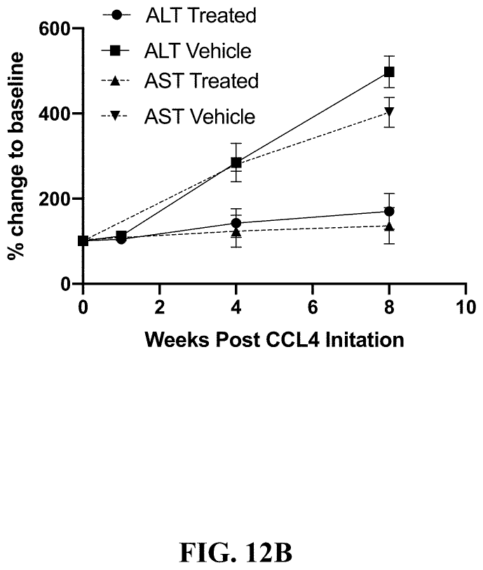

In this prophetic example, use of engineered myeloid cells in the treatment of liver fibrosis is demonstrated by using an experimental animal model. Liver fibrosis in induced in mice by repeated injection of carbon tetrachloride (CCL4) as shown in FIG. 12A. Materials: CCl4, OptiPrep, 4-MP and type I Collagenase are obtained from Sigma-Aldrich (St. Louis, Mo.). Percoll and DNase I are purchased from GE Healthcare (Buckinghamshire, United Kingdom) and Roche (Indianapolis, Ind.), respectively. Induction of Liver Fibrosis in Mice For the induction of CCl4-mediated liver fibrosis, age-matched (8-week-old) male C57BL/6J mice are administered intraperitoneal injections of CCl4 (20% CCl4 olive oil solution, 2 ml/kg of animal body weight) three times per week for 2 weeks (FIG. 12A). Mice in the control group received an equal volume of olive oil (vehicle). Mice are bled weekly to examine ALT and AST levels. Engineered cells are prepared as follows: myeloid cells are engineered to express the fibrosis mRNA construct having the components as demonstrated in the upper panel of FIG. 12A. Treatment of animals is performed using 1 million cells on days 10, 12, 14, 16, 18 and 20.

Prophetic data are shown in FIG. 12B, assessing the liver function by liver enzymes ALT and AST levels.

Additionally, myeloid cells can be programmed to address different stages of fibrosis. Normal liver, fatty liver (NAFL) and NASH can be staged by a series of markers, e.g., adiponectin, Leptin, cytokeratin-18 fragments, adipocyte fatty acid-binding protein; oxidative stress markers such as fibroblast growth factor 21 and thioredoxin, copper to zinc superoxide dismutase, glutathione peroxidase, hyaluronic acid, procollagen III, laminin, AST/ALT ratio, APRI, BAAT score, BARD score, ELF, FIB-4 index among others. These parameters in turn contribute to the disease score determination (or clinical mean score) determination for liver fibrosis.

Fibrosis and cyrrhosis constructs can be engineered to deliver the correct “signals” to reverse disease and can be made to be stage specific.

### Example 9: Engineered Myeloid Cells for Treatment of Graft Versus Host Disease (GVHD)

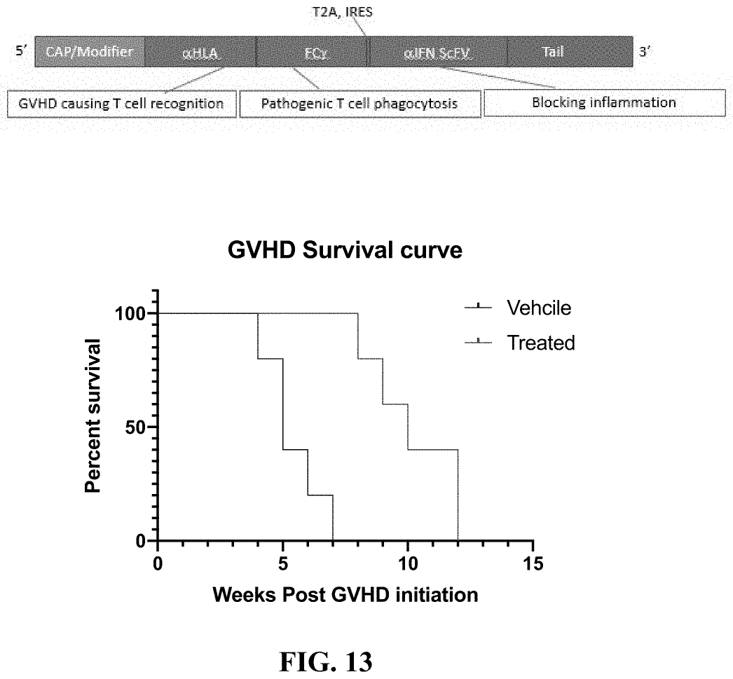

In this prophetic example, use of engineered myeloid cells in the treatment of GVHD is demonstrated by using an experimental animal model. Specific constructs comprise a sequence for anti-HLA for reducing T cell recognition, and/or a sequence encoding an Fc gamma region for inducing phagocytosis of pathogenic T cell, and an anti-IFN antibody or scFv for reducing inflammation by interferon activity (FIG. 13, top panel).

Male NSG mice are purchased from Jackson Laboratory (Bar Harbor, Me.). Animals are maintained in a pathogen-free animal facility. All of the animals received humane care according to the criteria outlined in the Guide for the Care and Use of Laboratory Animals published by National Institutes of Health.

GVHD induction: GVHD is induced through the infusion of activated T cells

Disease monitoring: Disease progression is monitored weekly through body weight. When animals lost 10% of body weight treatment was initiated.

Engineered cells—Myeloid cells are engineered to express the fibrosis mRNA construct. Treatment of animals is performed using 1 million cells weekly for 5 weeks, once animals showed 10% weight loss A prophetic exemplary data showing survival curve in animals receiving the myeloid cell therapy and vehicle is demonstrated in the graph.

These results demonstrate that the myeloid cells described herein can be successfully used in the development of highly efficacious tolerogenic cell therapy.

1-235. (canceled)

236. A pharmaceutical composition comprising:
(A) a pharmaceutically acceptable excipient, diluent or carrier; and
(B) (I) an exogenously modified population of monocytes, wherein the exogenously modified population of monocytes comprises a recombinant polynucleotide encoding a polypeptide comprising an autoantigenic peptide; or
(II) a composition comprising: (a) a recombinant polynucleotide encoding a polypeptide comprising an autoantigenic peptide; and (b) an agent that modifies a monocyte of a human subject when administered the pharmaceutical composition; such that the monocyte of the human subject are engulfed and/or recognized as apoptotic by APCs of the human subject.

237. The pharmaceutical composition of claim 236, wherein the exogenously modified population of monocytes are phagocytosed, engulfed and/or recognized as apoptotic by antigen presenting cells (APCs) of human subject administered the pharmaceutical composition.

238. The pharmaceutical composition of claim 236, wherein the recombinant polynucleotide in B(I) or B(II) comprises a first sequence encoding one or more autoantigenic peptides; and one or more additional sequences encoding one or more immune regulatory agents.

239. The pharmaceutical composition of claim 238, wherein each of the one or more autoantigenic peptides comprise an autoantigenic epitope.

240. The pharmaceutical composition of claim 236, wherein the autoantigenic peptide is unknown at the time of administration.

241. The pharmaceutical composition of claim 238, wherein the one or more immune regulatory agents is selected from a group consisting of TGF beta, IL10, PD1 and PDL1.

242. The pharmaceutical composition of claim 236, wherein the agent that modifies monocytes of a human subject is selected from a group consisting of TGF beta, IL10, PD1 and PDL1.

243. The pharmaceutical composition of claim 236, wherein the exogenously modified population of monocytes do not present the autoantigenic peptide, and wherein the polypeptide comprising the autoantigenic peptide lacks a secretory sequence.

244. The pharmaceutical composition of claim 236, wherein the polypeptide comprising the autoantigenic peptide is a full-length protein.

245. The pharmaceutical composition of claim 236, wherein the exogenously modified population of monocytes comprises exogenously modified monocytes that are apoptotic.

246. The pharmaceutical composition of claim 236, wherein the polypeptide comprising the autoantigenic peptide comprises a fusion protein, wherein the fusion protein comprises an antigen enhancer selected from the group consisting of LAMP-1/2, hsp110 and grp170, hsp70, hsp65, rab7 GTPas, PSGL-1/mIgG2b, macrophage mannose receptor (MMR), and dendritic cell-specific intercellular adhesion molecule-3 grabbing non-integrin (DC-SIGN) and a MHC class I trafficking signal.

247. The pharmaceutical composition of claim 236, wherein the autoantigenic peptide sequence and the antigen enhancer sequence or a fragment thereof are separated by a cleavable peptide sequence.

248. The pharmaceutical composition of claim 236, wherein the polypeptide comprising the autoantigenic peptide comprises an autoantigenic peptide from a protein selected from the group consisting of Gliadin, Barley, Hordein, MBP, MOG, PLP. Insulin, Pro-insulin, IGRP, Casein, ligand for a drug-neutralizing antibody, Factor VIII and a combination thereof.

249. The pharmaceutical composition of claim 236, wherein the recombinant polynucleotide is mRNA.

250. The pharmaceutical composition of claim 236, wherein the recombinant polynucleotide is DNA vector, wherein the DNA vector is a bacterial vector, a lentiviral vector, an adenoviral vector or an adeno-associated viral vector.

251. The pharmaceutical composition of claim 236, wherein the agent comprises an apoptosis-inducing agent.

252. The pharmaceutical composition of claim 251, wherein the agent is an agent that (i) crosslinks lipids; (ii) cross-links cell membrane components; and/or (iii) binds with double-stranded DNA and inhibits RNA synthesis of the monocytes.

253. The pharmaceutical composition of claim 251, wherein the agent is an acrylamide, a β carboline alkaloid, anthracycline, carvacrol, p-cymene, doxorubicin, daunorubicin (DNR), idarubicin (IDA), or camptothecin (CAM), blasticidin, or cycloheximide, 1-ethyl-3-(3-dimethylaminopropyl)-carbodiimide (EDC) or actinomycin D.

254. The pharmaceutical composition of claim 251, wherein the agent is 1-ethyl-3-(3-dimethylaminopropyl)-carbodiimide (EDC).

255. The pharmaceutical composition of 236, wherein the exogenously modified population of monocytes comprises Annexin V positive cells, and is an exogenously modified population of CD14+CD16+ cells, CD14dimCD16+ cells, CD14−CD16+ cells.

256. A method of treating or inducing immune tolerance to an autoantigenic peptide in a human subject with an immune-mediated disease or condition, the method comprising administering to the human subject the pharmaceutical composition of claim 236.
